(19) United States
(12) Patent Application Publication
(10) Pub. No.: US 2023/0132604 A1
(43) Pub. Date: May 4, 2023

(54) PHARMACEUTICAL COMPOSITION, PREPARATION METHOD THEREFOR AND USE THEREOF

(71) Applicants: Keymed Biosciences Co., Ltd, Chengdu (CN); Shanghai Lingyue Biopharma Co., Ltd, Shanghai (CN)

(72) Inventors: Juntao Yu, Shanghai (CN); Gang XU, Chengdu (CN); Bo CHEN, Chengdu (CN)

(21) Appl. No.: 17/780,852

(22) PCT Filed: Nov. 13, 2020
(86) PCT No.: PCT/CN2020/128820

§ 371 Date: May 27, 2022

(30) Foreign Application Priority Data
Nov. 29, 2019 (CN) 201911207085.X

(51) Int. Cl. C07K 16/28; A61P 35/00

(52) U.S. Cl. CPC C07K 16/2896; A61P 35/00; C07K 2317/24; C07K 2317/31; A61K 39/00

(57) ABSTRACT

The present disclosure relates to an antibody that binds to the CD38 protein or an antigen-binding portion thereof, a preparation method therefor and the use thereof. The antibody can bind to human CD38 with high affinity, has an inhibitory effect on the CD38 enzyme, has a CDC, ADCC, and/or ADCP killing activity with regard to different tumor cells, and has an anti-tumor function. Moreover, the antibody does not cause red blood cell lysis.

### TECHNICAL FIELD

The present invention relates to a CD38 antibody, a preparation method and use thereof.

### BACKGROUND ART

CD38 is a 46-kDa type-II transmembrane glycoprotein with an intracellular N-terminal cytoplasmic domain containing 21 amino acid residues, a transmembrane domain containing 21 amino acids, and a C-terminus extracellular domain containing 258 amino acid residues. CD38 regulates the migration and receptor-mediated adhesion through interaction with CD31 or hyaluronic acid, and in addition, CD38 has extracellular enzyme activity and is involved in the production of nucleotide metabolites, controlling intracellular calcium storage. CD38 is expressed at relatively low levels in some normal tissues, such as bone marrow and lymphoid cells, and in some non-hematopoietic cells; in contrast, it is highly expressed in plasma cells and some malignant cells, including multiple myeloma (MM) and chronic lymphocytic leukemia (CLL), thus CD38 is a target molecule for targeted multiple myeloma cell therapy due to the differences in expression abundance.

Multiple myeloma is considered a cancer of plasma cells, characterized by the accumulation of malignant cells in the bone marrow and the production of monoclonal immunoglobulins (M protein). Although the median overall survival of patients has improved significantly with the introducing of new therapies in recent years, patients typically relapse and develop resistance within one year. Daratumumab, a monoclonal antibody (mAb) that targets CD38, has been approved to be marketed for the treatment of relapsed/refractory multiple myeloma; several other mAbs in clinical trials such as isatuximab, MOR202, and TAK-079 target indications including newly diagnosed multiple myeloma in addition to relapsed/refractory multiple myeloma. CD38 antibodies also show good therapeutic effects in other malignancies, such as NK/T cell lymphoma, T cell acute lymphoblastic leukemia and immunoglobulin light chain amyloidosis.

Rheumatoid arthritis (RA) is an autoimmune disease in which a large number of B lymphocytes proliferate in the diseased synovial membrane. Flow cytometry analysis showed that the expression of CD38+, CD3+CD38+ and CD56+ CD38+ subsets in the peripheral blood of RA patients was significantly increased, and the expression of CD38+ cells was significantly correlated with rheumatoid factor (RF) levels in RA patients; immunohistochemical results showed that CD38 was specifically and highly expressed in the synovial membrane of RA patients; the ELISA experiment demonstrated that after siRNA interference with CD38 gene, the expression levels of IL-1α and IL-β in RASF medium are significantly decreased, and the increased expression of CD38 gene may be involved in the immune activation of RA patients (Expression of CD38 in peripheral blood and synovial tissue from patients with rheumatoid arthritis. Yue Longtao, et al. Current Immunology, pp. 89-93, 2014, No. 2). CD38 is significantly elevated in peripheral blood from patients with other autoimmune diseases such as systemic lupus erythematosus (SLE), and plays an important role in the occurrence and development of SLE. Although a variety of CD38 antibodies have shown very significant anti-tumor effects in vitro or preclinical animal studies, significant heterogeneity has been observed in clinical studies in the therapeutic effects and duration of response of the different antibodies, which may be related to the mechanism of action of the antibodies. For example, NK cells in the blood of patients treated with CD38 antibodies are rapidly depleted, and antibodies that primarily utilize antibody-dependent cellular cytotoxicity (ADCC) may be limited in function due to the lack of effector cells; antibodies with another mechanism of action (e. g., daratumumab has complement-dependent cytotoxicity, CDC) have been shown to be superior to other CD38 antibodies in clinical studies in overall response rate and survival time. It has been studied that CDC efficacy of an antibody is improved by introducing a mutation into IgG1 Fc or replacing the IgG1 hinge region with the IgG3 hinge region. Recently, it has also been studied that the mutation of IgG 1 Fc allows the monoclonal antibody to form a hexamer, which effectively enhances the killing efficacy of CD38 antibodies, but brings some difficulties in production and development.

### SUMMARY OF THE INVENTION

The present inventors immunized mice with CD38 recombinant protein, and obtained a variety of novel high-affinity CD38 antibodies with the strongest CDC, ADCC, and antibody-dependent cellular phagocytosis (ADCP) activity of the similar antibodies, as well as the ability to inhibit CD38 extracellular enzyme activity and induce apoptosis under Fc cross-linking conditions. The present inventor further introduced new mutations in the Fc segment of the antibody, and the tumor killing efficiency of the novel CD38 antibody is far higher than that of the similar antibodies on the market at present. In one aspect, the present disclosure provides an antibody or antigen-binding portion thereof that binds to a CD38 protein.

In one aspect, the present disclosure provides a nucleic acid encoding the antibody or antigen-binding portion thereof according to the preceding aspect.

In one aspect, the present disclosure provides a vector comprising the nucleic acid according to the preceding aspect.

In one aspect, the present disclosure provides a cell comprising the vector according to the preceding aspect.

The antibody or antigen-binding portion thereof according to any one of the preceding aspects is provided, wherein the antibody or antigen-binding portion thereof is humanized. In one aspect, the present disclosure provides a pharmaceutical composition or kit comprising an antibody or antigen-binding portion thereof or nucleic acids encoding the same according to any of the preceding aspects and a pharmaceutically acceptable carrier.

In one aspect, the present disclosure provides a method of treating the CD38-related disorders comprising the steps of administering to the mammal a therapeutically effective amount of an antibody or antigen-binding fragment thereof, a nucleic acid molecule, a vector, a cell and/or a pharmaceutical compositions according to any of the preceding aspects.

Use of the antibody or antigen-binding fragment thereof, the nucleic acid molecule, the vector, the cell and/or the pharmaceutical composition of any of the preceding aspects in the manufacture of a medicament or kit for the treatment of a CD38-related disorder in a mammal.

The antibodies may bind to human CD38 with high affinity, have an inhibitory effect on the CD38 enzyme, have CDC, ADCC, and/or ADCP killing activity on different tumor cells, and have an anti-tumor function. Also, the antibody does not cause lysis of red blood cells.

### BRIEF DESCRIPTION OF THE DRAWINGS

FIG. 1 illustrates the recombinant expression of human CD38 extracellular domain protein in HEK 293 cells;

FIG. 2 illustrates flow cytometry analysis of human CD38/CHO stably transfected cells;

FIG. 3-1 illustrates CDC effects of chimeric antibodies on Duadi cells;

FIG. 3-2 illustrates CDC effects of chimeric antibodies on Duadi cells;

FIG. 4-1 illustrates inhibition of chimeric antibodies on CD38 enzymatic activity;

FIG. 4-2 illustrates inhibition of chimeric antibodies on CD38 enzymatic activity;

FIG. 5 illustrates ADCC killing activity of chimeric antibodies on Duadi cells;

FIG. 6 illustrates ADCP killing activity of chimeric antibodies on Duadi cells;

FIG. 7-1 illustrates CDC effects of different humanized antibodies on Daudi cells;

FIG. 7-2 illustrates CDC effects of different humanized antibodies on Daudi cells;

FIG. 8-1 illustrates ELISA affinity assay;

FIG. 8-2 illustrates binding of anti-CD38 humanized antibody to Daudi cells;

FIG. 8-3 illustrates binding of anti-CD38 humanized antibody to NALM-6 cells;

FIG. 8-4 illustrates binding of anti-CD38 humanized antibody to Ramos.2G6.4C10 cells;

FIG. 8-5 illustrates binding of anti-CD38 humanized antibody to TALL-1 cells;

FIG. 9 illustrates expression of CD38 on different tumor cells;

FIG. 10-1 illustrates CDC effects of anti-CD38 humanized antibody on Ramos.2G6.4C10;

FIG. 10-2 illustrates CDC effects of anti-CD38 humanized antibody on Daudi;

FIG. 10-3 illustrates CDC effects of anti-CD38 humanized antibody on TALL-1;

FIG. 11-1 illustrates ADCC effects of anti-CD38 humanized antibody on Ramos.2G6.4C10;

FIG. 11-2 illustrates ADCC effects of anti-CD38 humanized antibody on NALM-6;

FIG. 11-3 illustrates ADCC effects of anti-CD38 humanized antibody on RPMI-8226;

FIG. 11-4 illustrates ADCC effects of anti-CD38 humanized antibody on CCRF-CEM;

FIG. 11-5 illustrates ADCC effects of anti-CD38 humanized antibody on Daudi;

FIG. 11-6 illustrates ADCC effects of anti-CD38 humanized antibody on TALL-1;

FIG. 11-7 illustrates ADCC effects of anti-CD38 humanized antibody on MM.1S;

FIG. 11-8 illustrates ADCC effects of anti-CD38 humanized antibody on NCI-H929;

FIG. 11-9 illustrates ADCC effects of anti-CD38 humanized antibody on Raji;

FIG. 12-1 illustrates ADCP effects of anti-CD38 humanized antibody on Daudi;

FIG. 12-2 illustrates ADCP effects of anti-CD38 humanized antibody on TALL-1;

FIG. 12-3 illustrates ADCP effects of anti-CD38 humanized antibody on Ramos.2G6.4C10;

FIG. 12-4 illustrates ADCP effects of anti-CD38 humanized antibody on Raji;

FIG. 12-5 illustrates ADCP effects of anti-CD38 humanized antibody on NALM-6;

FIG. 13 illustrates the analysis of the inhibition of humanized CD38 antibodies on CD38 enzymatic activity;

FIG. 14 illustrates Daudi cell apoptosis induced by humanized CD38 antibody;

FIG. 15 illustrates human erythrocyte lysis assay induced by humanized CD38 antibody; and

FIG. 16 illustrates effect of CD38 antibody on growth of human B-cell lymphoma Daudi subcutaneous xenografts.

### DETAILED DESCRIPTION OF THE INVENTION

### I. Definitions

In the present invention, unless otherwise specified, scientific and technical terms used herein have the meanings commonly understood by those skilled in the art. Also, as used herein, protein and nucleic acid chemistry, molecular biology, cell and tissue culture, microbiology, immunology, and laboratory procedures used herein are terms and routine procedures widely used in the corresponding fields. Meanwhile, in order to better understand the present invention, definitions and explanations of related terms are provided below.

In one aspect, provided herein are antibodies (e. g., monoclonal antibodies) that specifically bind to CD38 and antigen-binding fragments thereof. In particular aspects, provided herein are anti-CD38 monoclonal antibodies that specifically bind to CD38, wherein the anti-CD38 antibodies include variants of the parent antibodies. In particular aspects, provided herein are antibodies that specifically bind to CD38 (e. g., human CD38). In particular aspects, provided herein are anti-CD38 antibodies comprising modifications in one or more amino acid residues (e. g., 5-13 amino acid substitutions in the framework region of the heavy chain variable region) that retain affinity for an antigen as compared to the parent antibody without the modification.

The term “about” or “approximately” as used herein means within plus or minus 10% of a given value or range, unless otherwise specified. Where integers are required, the term refers to integers within plus or minus 10% of a given value or range, rounded up or down to the nearest integer.

The phrase “substantially identical” with respect to an antibody chain polypeptide sequence may be construed as an antibody chain exhibiting at least 60%, 65%, 70%, 75%, 80%, 85%, 90%, 95%, 96%, 97%, 98%, 99% or more sequence identity to a reference polypeptide sequence. The term with respect to a nucleotide sequence may be construed as a sequence of nucleotides exhibiting at least about 60%, 65%, 70%, 75%, 80%, 85%, 90%, 95%, 96%, 97%, 98%, 99% or more sequence identity to the reference nucleotide sequence.

Sequence “identical” or “identity” has the recognized meaning in this field, and the percentage of sequence identity between two nucleic acid or polypeptide molecules or regions can be calculated using published techniques. Sequence identity can be measured along the entire length of a polynucleotide or polypeptide or along a region of the molecule (see, for example: Computational Molecular Biology, Lesk, A. M., ed., Oxford University Pres, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds, Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). While there are many methods of measuring identity between two polynucleotides or polypeptides, the term “identity” is well known to those skilled in the art (Carrillo, H. & Lipman, D SIAM J Applied Math 48: 1073 (1988)).

“Substitution” variants are those in which at least one amino acid residue in the native sequence has been removed and inserted by a different amino acid at the same position. The substitution may be single, with only one amino acid in the molecule being substituted; or may be multiple, with two or more amino acids being substituted in the same molecule. Multiple substitutions may be at consecutive positions. Likewise, an amino acid may be substituted with multiple residues, wherein such variants include both substitutions and insertions. “Insertion” variants are those in which one or more amino acids are inserted immediately adjacent to an amino acid at a particular position in a native sequence. By immediately adjacent amino acid is meant attached to the α-carboxy or α-amino functional group of the amino acid. “Deletion” variants are those in which one or more amino acids in the native amino acid sequence have been removed. Typically, one or two amino acids are deleted in a particular region of the molecules of deletion variants.

With respect to the variable domains of antibodies, the term “variable” refers to certain portions of related molecules that differ widely in sequence between antibodies and are used for specifically recognizing and binding to a specific target of a particular antibody. However, the variability is not uniformly distributed throughout the variable domain of the antibody. Variability is concentrated on three segments called complementarity determining regions (CDR; i.e., CDR1, CDR2, and CDR3) or hypervariable regions, all of which are located within the variable domains of the light and heavy chains. The more conserved portions within the variable domains are referred to as framework (FR) regions or framework sequences. Each variable domain of native heavy and light chains comprises four FR regions, predominantly in a β-sheet configuration, connected by three CDRs, which form loops connecting and in some cases forming part of the β-sheet structure. The CDRs of each chain are typically joined together by adjacent FR regions and aid in the formation of antibody target binding sites (epitopes or determinants) by means of CDR from other chains (see Kabat et al. Sequences of Proteins of Immunological Interest, national Institute of Health, bethesda, MD (1987)). As used herein, the immunoglobulin amino acid residue numbering is in accordance with the Kabat numbering scheme (Kabat et al.) for numbering amino acid residues in the immunoglobulin, unless otherwise indicated. One CDR may have the ability to specifically bind to a cognate epitope.

As used herein, an “antibody fragment” or “antigen-binding fragment” of an antibody refers to any portion of a full-length antibody that is less than full-length, but that comprises at least a portion of the variable region (e. g., one or more CDRs and/or one or more antibody binding sites) of the antibody that binds antigen, thus retaining binding specificity as well as at least a portion of the specific binding capacity of the full-length antibody. Thus, an antigen-binding fragment refers to an antibody fragment that comprises an antigen-binding portion that binds the same antigen as the antibody from which the antibody fragment was derived. Antibody fragments include antibody derivatives produced by enzymatic treatment of full-length antibodies, as well as synthetic derivatives, e. g., recombinant derivatives Antibodies include antibody fragments. Examples of antibody fragments include, but are not limited to, Fab, Fab′, F(ab′)₂, single chain Fv (scFv), Fv, dsFv, diabodies, Fd, and Fd′ fragments and other fragments, including modified fragments (see, e. g., Methods in Molecular Biology, Vol 207: Recombinant Antibodies for Cancer Therapy Methods and Protocols (2003); Chapter 1; p 3-25, Kipriyanov). The fragments may comprise multiple chains linked together, for example, by disulfide bonds and/or by peptide linkers. Antibody fragments generally comprise at least or about 50 amino acids, and typically at least or about 200 amino acids. Antigen-binding fragments include any antibody fragment that, when inserted into an antibody framework (e. g., by displacement of the corresponding region), results in an antibody that immunospecifically binds (i. e., exhibiting a Ka of at least or at least about 10⁷-10⁸M−1) an antigen. A “functional fragment” or “analog of an anti-CD38 antibody” is a fragment or analog that prevents or substantially reduces the ability of the receptor to bind a ligand or initiate signal transduction. As used herein, functional fragments generally have the same meaning as “antibody fragments” and, in the case of antibodies, can refer to fragments that prevent or substantially reduce the ability of the receptor to bind a ligand or initiate signal transduction, e. g., Fv, Fab, F(ab′)₂, and the like. An “Fv” fragment consists of a dimer (V_{H}-V_{L} dimer) of a variable domain of a heavy chain and a variable domain of a light chain formed by non-covalent association. In this configuration, the three CDRs of each variable domain interact to define a target binding site on the surface of the V_{H}-V_{L} dimer, as is the case with intact antibodies. The six CDRs together confer target binding specificity to the intact antibody. However, even a single variable domain (or half of an Fv comprising only three target-specific CDRs) may still have the ability to recognize and bind to targets.

As used herein, the term “bispecific antibody (BsAb)” refers to an antibody and/or antigen-binding molecule capable of specifically binding to two different antigenic determinants. Generally, a bispecific antibody and/or antigen-binding molecule comprises two antigen-binding sites, each of which is specific for different antigenic determinants. In some embodiments, the bispecific antibody and/or antigen binding molecule is capable of binding two antigenic determinants simultaneously, particularly two antigenic determinants expressed on two different cells.

As used herein, “monoclonal antibody” refers to a population of identical antibodies, meaning that each individual antibody molecule in the population of monoclonal antibodies is identical to another antibody molecule. This property is in contrast to the property of a population of polyclonal antibodies comprising antibodies having a plurality of different sequences. Monoclonal antibodies can be prepared by a number of well-known methods (Smith et al. (2004) J. Clin. Pathol.57, 912-917; and Nelson et al., J Clin Pathol (2000), 53, 111-117). For example, monoclonal antibodies can be prepared by immortalizing B cells, for example, by fusing B cells with myeloma cells to generate hybridoma cell lines or by infecting B cells with a virus such as EBV. Recombinant techniques can also be used to prepare antibodies from clonal populations of host cells in vitro by transforming host cells with plasmids carrying artificial sequences encoding the nucleotides of the antibodies.

As used herein, the term “hybridoma” or “hybridoma cell” refers to a cell or cell line (typically a myeloma or lymphoma cell) resulting from the fusion of antibody-producing lymphocytes and non-antibody-producing cancer cells. As known to those of ordinary skill in the art, hybridomas can be propagated and continuously supplied to produce a particular monoclonal antibody. Methods for producing hybridomas are known in the art (see, e. g., Harlow & Lane, 1988). When referring to the term “hybridoma” or “hybridoma cell”, it also includes subclones and progeny cells of hybridomas.

As used herein, a full-length antibody is an antibody having two full-length heavy chains (e. g. VH-CH1-CH2-CH3 or VH-CH1-CH2-CH3-CH4) and two full-length light chains (VL-CL) and a hinge region, e. g., an antibody naturally produced by an antibody secreting B cell as well as a synthetically produced antibody having the same domain.

The term “chimeric antibody” refers to an antibody in which the variable region sequences are derived from one species and the constant region sequences are derived from another species, such as an antibody in which the variable region sequences are derived from a mouse antibody and the constant region sequences are derived from a human antibody.

A “humanized” antibody refers to a form of non-human (e. g., mouse) antibody that is a chimeric immunoglobulin, immunoglobulin chain or fragment thereof (e. g., Fv, Fab, Fab′, F(ab′)₂ or other antigen-binding subsequence of an antibody), containing minimal sequence derived from a non-human immunoglobulin. Preferably, the humanized antibody is a human immunoglobulin (recipient antibody) in which residues from a complementarity determining region (CDR) of the recipient antibody are replaced by CDR residues from a non-human species (donor antibody) such as mouse, rat, or rabbit having the desired specificity, affinity, and capacity.

Furthermore, in humanization it is also possible to mutate amino acid residues within the CDR1, CDR2 and/or CDR3 regions of VH and/or VL, thereby improving one or more binding properties (e. g., affinity) of the antibody. Mutations can be introduced, e. g. by PCR-mediated mutagenesis, and their effect on antibody binding or other functional properties can be assessed using the in vitro or in vivo assays described herein. Typically, conservative mutations are introduced. Such mutations may be amino acid substitutions, additions, or deletions. In addition, mutations within CDR typically do not exceed one or two. Thus, humanized antibodies of the present invention also encompass antibodies comprising one or two amino acid mutations within CDR.

As used herein, the term “CDR” refers to a complementarity-determining region known to have three CDRs per heavy and light chain of an antibody molecule. CDR is also known as a hypervariable region and is present in the variable region of each of the heavy and light chains of an antibody with a very high site of variability in the primary structure of CDR. In the present specification, the CDR of the heavy chain is represented by CDR1, CDR2, CDR3 from the amino terminus of the amino terminal sequence of the heavy chain, and the CDR of the light chain is represented by CDR1, CDR2, CDR3 from the amino terminus of the amino terminal sequence of the light chain. These sites are adjacent to each other in the tertiary structure and determine the specificity of the antigen to which the antibody binds.

As used herein, the term “epitope” refers to any antigenic determinant on an antigen to which the paratope of an antibody binds. Epitopic determinants usually comprise chemically active surface types of molecules, such as amino acids or sugar side chains, and usually have specific three-dimensional structural characteristics as well as specific charge characteristics.

As used herein, “specific binding” or “immunospecifically binding” with respect to an antibody or antigen-binding fragment thereof is used interchangeably herein and refers to the ability of an antibody or antigen-binding fragment to form one or more non-covalent bonds with the same antigen through non-covalent interactions between the antibody and the antibody binding site of the antigen. The antigen may be an isolated antigen or present in a tumor cell. Typically, an antibody that immunospecifically binds (or specifically binds) to an antigen is one that binds to the antigen with an affinity constant Ka of about or 1×10⁷M⁻¹ or 1×10⁸M⁻¹ or more (or a dissociation constant (Kd) of 1×10⁻⁷M or 1×10⁻⁸M or less). Affinity constants can be determined by standard kinetic methods for antibody reactions, e. g., immunoassays, surface plasmon resonance (SPR) (Rich and Myszka (2000) Curr. Opin. Biotechnol 11:54; Englebienne (1998) Analyst. 123:1599), isothermal titration calorimetry (ITC), or other kinetic interaction assays known in the art (see, e. g., Paul, ed., Fundamental Immunology, 2nd ed., Raven Press, New York, pages 332-336 (1989); see also U.S. Pat. No. 7,229,619) which describes an exemplary SPR and ITC method for calculating the binding affinity of an antibody. Instruments and methods for real-time detecting and monitoring the rate of binding are known and commercially available (see, BiaCore 2000, Biacore AB, Upsala, Sweden and GE Healthcare Life Sciences; Malmqvist (2000) Biochem. Soc. Trans. 27:335).

As used herein, the terms “polynucleotide” and “nucleic acid molecule” refer to an oligomer or polymer comprising at least two linked nucleotides or nucleotide derivatives, including deoxyribonucleic acid (DNA) and ribonucleic acid (RNA), typically linked together by phosphodiester bonds. As used herein, the term “nucleic acid molecule” is intended to include DNA molecules and RNA molecules. The nucleic acid molecule may be single-stranded or double-stranded, and may be cDNA.

As used herein, an isolated nucleic acid molecule is a nucleic acid molecule isolated from other nucleic acid molecules present in the natural source of the nucleic acid molecule. An “isolated” nucleic acid molecule, such as a cDNA molecule, can be substantially free of other cellular material or culture medium when prepared by recombinant techniques, or substantially free of chemical precursors or other chemical components when chemically synthesized. Exemplary isolated nucleic acid molecules provided herein include isolated nucleic acid molecules encoding a provided antibody or antigen-binding fragment.

As used herein, “operably linked” with respect to a nucleotide sequence, region, element or domain means that the nucleic acid regions are functionally related to one another. For example, a promoter may be operably linked to a nucleic acid encoding a polypeptide such that the promoter regulates or mediates transcription of the nucleic acid.

Also provided are “conservative sequence modifications” of the sequences set forth in the sequence listings described herein, i.e., nucleotide and amino acid sequence modifications that do not eliminate binding of an antibody encoded by a nucleotide sequence or containing an amino acid sequence to an antigen. These conservative sequence modifications include substitutions of conservative nucleotide and amino acid and additions and deletions of nucleotide and amino acid. For example, modifications can be introduced into the sequence listings described herein by standard techniques known in the art, such as site-directed mutagenesis and PCR-mediated mutagenesis. Conservative sequence modifications include conservative amino acid substitutions in which an amino acid residue is replaced with an amino acid residue having a similar side chain Families of amino acid residues having similar side chains have been defined in the art. These families include amino acids containing basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), non-polar side chains (e.g., alanine, valine, leucine, isoleucine, proline, phenylalanine, methionine), β-branched side chains (e.g., threonine, valine, isoleucine), and aromatic side chains (e.g., tyrosine, phenylalanine, tryptophan, histidine). Thus, a predicted nonessential amino acid residue in an anti-CD38 antibody is preferably replaced with another amino acid residue from the same side chain family. Methods for identifying conservative substitutions of nucleotides and amino acids that do not eliminate antigen binding are well known in the art (see, e. g., Brummell et al., Biochem. 32: 1180-1187 (1993); Kobayashi et al., Protein Eng. 12(10): 879-884 (1999); Burks et al., Proc. Natl. Acad. Sci. USA 94: 412-417 (1997)).

Alternatively, in another embodiment, mutations can be randomly introduced along all or a portion of the anti-CD38 antibody coding sequence, e.g., by saturation mutagenesis, and the resulting modified anti-CD38 antibodies can be screened for improved binding activity.

As used herein, “expression” refers to a process of producing a polypeptide by transcription and translation of a polynucleotide. The level of expression of a polypeptide can be assessed using any method known in the art, including, for example, methods of determining the amount of polypeptide produced from a host cell. Such methods may include, but are not limited to, quantitation of polypeptides in cell lysates by ELISA, gel electrophoresis followed by Coomassie blue staining, Lowry protein assay, and Bradford protein assay.

As used herein, a “host cell” is a cell for receiving, maintaining, replicating, and expanding a vector. The host cell may also be used to express a polypeptide encoded by the vector. The nucleic acid contained in the vector replicates during the host cell division, thereby amplifying the nucleic acid. The host cell may be a eukaryotic cell or a prokaryotic cell. Suitable host cells include, but are not limited to, CHO cells, various COS cells, HeLa cells, HEK cells such as HEK 293 cells.

As used herein, a “vector” is a replicable nucleic acid from which one or more heterologous proteins can be expressed when the vector is transformed into an appropriate host cell. References to vectors include those into which a nucleic acid encoding a polypeptide or fragment thereof may be introduced, typically by restriction digestion and ligation. References to vectors also include those comprising a nucleic acid encoding a polypeptide. Vectors are used to introduce a nucleic acid encoding a polypeptide into a host cell, to amplify the nucleic acid, or to express/display the polypeptide encoded by the nucleic acid. The vector generally remains episomal, but can be designed to allow integration of a gene or portion thereof into the chromosome of the genome. Also contemplated are vectors for artificial chromosomes, such as yeast artificial vectors and mammalian artificial chromosomes. The selection and use of such vehicles is well known to those skilled in the art.

As used herein, a vector also includes a “virus vector” or a “viral vector”. A viral vector is an engineered virus that is operably linked to a foreign gene to transfer (as a vehicle or shuttle) the foreign gene into a cell.

As used herein, an “expression vector” includes a vector capable of expressing a DNA operably linked to regulatory sequences, such as a promoter region, capable of affecting the expression of such DNA fragments. Such additional fragments may include promoter and terminator sequences, and optionally may include one or more origins of replication, one or more selectable markers, enhancers, polyadenylation signals, etc. Expression vectors are typically derived from plasmid or viral DNA, or may contain elements of both. Thus, an expression vector refers to a recombinant DNA or RNA construct, such as a plasmid, phage, recombinant virus or other vectors, which results in the expression of a cloned DNA when introduced into an appropriate host cell. Appropriate expression vectors are well known to those skilled in the art and include expression vectors that are replicable in eukaryotic and/or prokaryotic cells as well as expression vectors that remain episomal or that are integrated into the host cell genome.

As used herein, “treating” a subject with a disease or condition means that the subject's symptoms are partially or completely alleviated or remain unchanged after the treatment. Thus, treatment includes prevention, treatment and/or cure. The prevention refers to preventing the underlying disease and/or preventing the worsening of symptoms or the progression of the disease. The treatment also includes any antibody or antigen-binding fragment thereof provided and any pharmaceutical use of the compositions provided herein.

As used herein, “therapeutic effect” refers to an effect resulting from treatment of a subject that alters, typically improves or ameliorates a symptom of a disease or condition, or cures a disease or condition.

As used herein, a “therapeutically effective amount” or “therapeutically effective dose” refers to an amount of a substance, compound, material, or composition comprising a compound that is at least sufficient to produce a therapeutic effect after being administered to a subject. Thus, it is an amount necessary to prevent, cure, ameliorate, block, or partially block the symptoms of a disease or disorder.

As used herein, a “prophylactically effective amount” or “prophylactically effective dose” refers to an amount of a substance, compound, material, or composition comprising a compound that, when administered to a subject, has the desired prophylactic effect, e. g., preventing or delaying the occurrence or recurrence of a disease or condition, reducing the likelihood of the occurrence or recurrence of a disease or condition. A fully prophylactically effective dose need not occur by administration of one dose, and may occur only after administration of a series of doses. Thus, a prophylactically effective amount may be administered one or more times.

As used herein, the term “patient” refers to a mammal, such as a human.

### II. Detailed Description of Embodiments

In one aspect, the present disclosure provides an antibody or antigen-binding portion thereof that binds to CD38, comprising heavy chain CDRs selected from the amino acid sequences of SEQ ID NOs: 2-4, 12-14, 22-24, 32-34, 42-44, 52-54, 62-64, 72-74, 82-84, 92-94, 102-144, 112-114, 122-124, 132-34, 142-144, 152-154, 162-164, 172-174, 182-184, 187-189, 192-194, 197-199, 202-204, 207-209, 212-214, 217-219, 222-224, 227-229, 232-234, or any variant thereof, and/or light chain CDRs selected from the amino acid sequences of SEQ ID NOs: 7-9, 17-19, 27-29, 37-39, 47-49, 57-59, 67-69, 77-79, 87-89, 97-99, 107-109, 117-119, 127-129, 137-139, 147-149, 157-159, 167-169, 177-179, 237-239, 242-244, 247-249, 252-254, 257-259, 262-264, 267-269, or any variant thereof.

The antibody or antigen-binding portion thereof according to the preceding aspect comprises a heavy chain CDR1 selected from the amino acid sequences of SEQ ID NOs: 2, 12, 22, 32, 42, 52, 62, 72, 82, 92, 102, 112, 122, 132, 142, 152, 162, 172, 182, 187, 192, 197, 202, 207, 212, 217, 222, 227, 232, or any variant thereof, a heavy chain CDR2 selected from the amino acid sequences of SEQ ID NOs: 3, 13, 23, 33, 43, 53, 63, 73, 83, 93, 103, 113, 123, 133, 143, 153, 163, 173, 183, 188, 193, 198, 203, 208, 213, 218, 223, 228, 233, or any variant thereof, and a heavy chain CDR3 selected from the amino acid sequences of SEQ ID NOs: 4, 14, 24, 34, 44, 54, 64, 74, 84, 94, 104, 114, 124, 134, 144, 154, 164, 174, 189, 194, 199, 204, 209, 214, 219, 224, 229, 234, or any variant thereof; and/or a light chain CDR1 selected from the amino acid sequences of SEQ ID NOs: 7, 17, 27, 37, 47, 57, 67, 77, 87, 97, 107, 117, 127, 137, 147, 157, 167, 177, 237, 242, 247, 252, 257, 262, 267, or any variant thereof, a light chain CDR2 selected from the amino acid sequences of SEQ ID NOs: 8, 18, 28, 38, 48, 58, 68, 78, 88, 98, 108, 118, 128, 138, 148, 158, 168, 178, 238, 243, 248, 253, 258, 263, 268, or any variant thereof, and a light chain CDR3 selected from the amino acid sequences of SEQ ID NOs: 9, 19, 29, 39, 49, 59, 69, 79, 89, 99, 109, 119, 129, 139, 149, 159, 169, 179, 239, 244, 249, 254, 259, 264, 269, or any variant thereof.

The antibody or antigen-binding portion thereof according to the preceding aspect comprises a CDR combination of heavy and light chains selected from the group consisting of:

(1) the heavy chain CDR1, CDR2, and CDR3 sequences including SEQ ID NOs: 2-4, respectively, and/or the light chain CDR1, CDR2, and CDR3 sequences including SEQ ID NOs: 7-9, respectively;

(2) the heavy chain CDR1, CDR2, and CDR3 sequences including SEQ ID NOs: 12-14, respectively, and/or the light chain CDR1, CDR2, and CDR3 sequences including SEQ ID NOs: 17-19, respectively;

(3) the heavy chain CDR1, CDR2, and CDR3 sequences including SEQ ID NOs: 22-24, respectively, and/or the light chain CDR1, CDR2, and CDR3 sequences including SEQ ID NOs: 27-29, respectively;

(4) the heavy chain CDR1, CDR2, and CDR3 sequences including SEQ ID NOs: 32-34, respectively, and/or the light chain CDR1, CDR2, and CDR3 sequences including SEQ ID NOs: 37-39, respectively;

(5) the heavy chain CDR1, CDR2, and CDR3 sequences including SEQ ID NOs: 42-44, respectively, and/or the light chain CDR1, CDR2, and CDR3 sequences including SEQ ID NOs: 47-49, respectively;

(6) the heavy chain CDR1, CDR2, and CDR3 sequences including SEQ ID NOs: 52-54, respectively, and/or the light chain CDR1, CDR2, and CDR3 sequences including SEQ ID NOs: 57-59, respectively;

(7) the heavy chain CDR1, CDR2, and CDR3 sequences including SEQ ID NOs: 62-64, respectively, and/or the light chain CDR1, CDR2, and CDR3 sequences including SEQ ID NOs: 67-69, respectively;

(8) the heavy chain CDR1, CDR2, and CDR3 sequences including SEQ ID NOs: 72-74, respectively, and/or the light chain CDR1, CDR2, and CDR3 sequences including SEQ ID NOs: 77-79, respectively;

(9) the heavy chain CDR1, CDR2, and CDR3 sequences including SEQ ID NOs: 82-84, respectively, and/or the light chain CDR1, CDR2, and CDR3 sequences including SEQ ID NOs: 87-89, respectively;

(10) the heavy chain CDR1, CDR2, and CDR3 sequences including SEQ ID NOs: 92-94, respectively, and/or the light chain CDR1, CDR2, and CDR3 sequences including SEQ ID NOs: 97-99, respectively;

(11) the heavy chain CDR1, CDR2, and CDR3 sequences including SEQ ID NOs: 102-104, respectively, and/or the light chain CDR1, CDR2, and CDR3 sequences including SEQ ID NOs: 107-109, respectively;

(12) the heavy chain CDR1, CDR2, and CDR3 sequences including SEQ ID NOs: 112-114, respectively, and/or the light chain CDR1, CDR2, and CDR3 sequences including SEQ ID NOs: 117-119, respectively;

(13) the heavy chain CDR1, CDR2, and CDR3 sequences including SEQ ID NOs: 122-124, respectively, and/or the light chain CDR1, CDR2, and CDR3 sequences including SEQ ID NOs: 127-129, respectively;

(14) the heavy chain CDR1, CDR2, and CDR3 sequences including SEQ ID NOs: 132-134, respectively, and/or the light chain CDR1, CDR2, and CDR3 sequences including SEQ ID NOs: 137-139, respectively;

(15) the heavy chain CDR1, CDR2, and CDR3 sequences including SEQ ID NOs: 142-144, respectively, and/or the light chain CDR1, CDR2, and CDR3 sequences including SEQ ID NOs: 147-149, respectively;

(16) the heavy chain CDR1, CDR2, and CDR3 sequences including SEQ ID NOs: 152-154, respectively, and/or the light chain CDR1, CDR2, and CDR3 sequences including SEQ ID NOs: 157-159, respectively;

(17) the heavy chain CDR1, CDR2, and CDR3 sequences including SEQ ID NOs: 162-164, respectively, and/or the light chain CDR1, CDR2, and CDR3 sequences including SEQ ID NOs: 167-169, respectively;

(18) the heavy chain CDR1, CDR2, and CDR3 sequences including SEQ ID NOs: 172-174, respectively, and/or the light chain CDR1, CDR2, and CDR3 sequences including SEQ ID NOs: 177-179, respectively;

(19) the heavy chain CDR1, CDR2, and CDR3 sequences including SEQ ID NOs: 182-184, respectively, and/or the light chain CDR1, CDR2, and CDR3 sequences including SEQ ID NOs: 237-239, respectively;

(20) the heavy chain CDR1, CDR2, and CDR3 sequences including SEQ ID NOs: 182-184, respectively, and/or the light chain CDR1, CDR2, and CDR3 sequences including SEQ ID NOs: 242-244, respectively;

(21) the heavy chain CDR1, CDR2, and CDR3 sequences including SEQ ID NOs: 182-184, respectively, and/or the light chain CDR1, CDR2, and CDR3 sequences including SEQ ID NOs: 247-249, respectively;

(22) the heavy chain CDR1, CDR2, and CDR3 sequences including SEQ ID NOs: 182-184, respectively, and/or the light chain CDR1, CDR2, and CDR3 sequences including SEQ ID NOs: 252-254, respectively;

(23) the heavy chain CDR1, CDR2, and CDR3 sequences including SEQ ID NOs: 182-184, respectively, and/or the light chain CDR1, CDR2, and CDR3 sequences including SEQ ID NOs: 257-259, respectively;

(24) the heavy chain CDR1, CDR2, and CDR3 sequences including SEQ ID NOs: 187-189, respectively, and/or the light chain CDR1, CDR2, and CDR3 sequences including SEQ ID NOs: 237-239, respectively;

(25) the heavy chain CDR1, CDR2, and CDR3 sequences including SEQ ID NOs: 187-189, respectively, and/or the light chain CDR1, CDR2, and CDR3 sequences including SEQ ID NOs: 242-244, respectively;

(26) the heavy chain CDR1, CDR2, and CDR3 sequences including SEQ ID NOs: 187-189, respectively, and/or the light chain CDR1, CDR2, and CDR3 sequences including SEQ ID NOs: 247-249, respectively;

(27) the heavy chain CDR1, CDR2, and CDR3 sequences including SEQ ID NOs: 187-189, respectively, and/or the light chain CDR1, CDR2, and CDR3 sequences including SEQ ID NOs: 252-254, respectively;

(28) the heavy chain CDR1, CDR2, and CDR3 sequences including SEQ ID NOs: 187-189, respectively, and/or the light chain CDR1, CDR2, and CDR3 sequences including SEQ ID NOs: 257-259, respectively;

(29) the heavy chain CDR1, CDR2, and CDR3 sequences including SEQ ID NOs: 192-194, respectively, and/or the light chain CDR1, CDR2, and CDR3 sequences including SEQ ID NOs: 237-239, respectively;

(30) the heavy chain CDR1, CDR2, and CDR3 sequences including SEQ ID NOs: 192-194, respectively, and/or the light chain CDR1, CDR2, and CDR3 sequences including SEQ ID NOs: 242-244, respectively;

(31) the heavy chain CDR1, CDR2, and CDR3 sequences including SEQ ID NOs: 192-194, respectively, and/or the light chain CDR1, CDR2, and CDR3 sequences including SEQ ID NOs: 247-249, respectively;

(32) the heavy chain CDR1, CDR2, and CDR3 sequences including SEQ ID NOs: 192-194, respectively, and/or the light chain CDR1, CDR2, and CDR3 sequences including SEQ ID NOs: 252-254, respectively;

(33) the heavy chain CDR1, CDR2, and CDR3 sequences including SEQ ID NOs: 192-194, respectively, and/or the light chain CDR1, CDR2, and CDR3 sequences including SEQ ID NOs: 257-259, respectively;

(34) the heavy chain CDR1, CDR2, and CDR3 sequences including SEQ ID NOs: 197-199, respectively, and/or the light chain CDR1, CDR2, and CDR3 sequences including SEQ ID NOs: 237-239, respectively;

(35) the heavy chain CDR1, CDR2, and CDR3 sequences including SEQ ID NOs: 197-199, respectively, and/or the light chain CDR1, CDR2, and CDR3 sequences including SEQ ID NOs: 242-244, respectively;

(36) the heavy chain CDR1, CDR2, and CDR3 sequences including SEQ ID NOs: 197-199, respectively, and/or the light chain CDR1, CDR2, and CDR3 sequences including SEQ ID NOs: 247-249, respectively;

(37) the heavy chain CDR1, CDR2, and CDR3 sequences including SEQ ID NOs: 197-199, respectively, and/or the light chain CDR1, CDR2, and CDR3 sequences including SEQ ID NOs: 252-254, respectively;

(38) the heavy chain CDR1, CDR2, and CDR3 sequences including SEQ ID NOs: 197-199, respectively, and/or the light chain CDR1, CDR2, and CDR3 sequences including SEQ ID NOs: 257-259, respectively;

(39) the heavy chain CDR1, CDR2, and CDR3 sequences including SEQ ID NOs: 202-204, respectively, and/or the light chain CDR1, CDR2, and CDR3 sequences including SEQ ID NOs: 237-239, respectively;

(40) the heavy chain CDR1, CDR2, and CDR3 sequences including SEQ ID NOs: 202-204, respectively, and/or the light chain CDR1, CDR2, and CDR3 sequences including SEQ ID NOs: 242-244, respectively;

(41) the heavy chain CDR1, CDR2, and CDR3 sequences including SEQ ID NOs: 202-204, respectively, and/or the light chain CDR1, CDR2, and CDR3 sequences including SEQ ID NOs: 247-249, respectively;

(42) the heavy chain CDR1, CDR2, and CDR3 sequences including SEQ ID NOs: 202-204, respectively, and/or the light chain CDR1, CDR2, and CDR3 sequences including SEQ ID NOs: 252-254, respectively;

(43) the heavy chain CDR1, CDR2, and CDR3 sequences including SEQ ID NOs: 202-204, respectively, and/or the light chain CDR1, CDR2, and CDR3 sequences including SEQ ID NOs: 257-259, respectively;

(44) the heavy chain CDR1, CDR2, and CDR3 sequences including SEQ ID NOs: 207-209, respectively, and/or the light chain CDR1, CDR2, and CDR3 sequences including SEQ ID NOs: 237-239, respectively;

(45) the heavy chain CDR1, CDR2, and CDR3 sequences including SEQ ID NOs: 207-209, respectively, and/or the light chain CDR1, CDR2, and CDR3 sequences including SEQ ID NOs: 242-244, respectively;

(46) the heavy chain CDR1, CDR2, and CDR3 sequences including SEQ ID NOs: 207-209, respectively, and/or the light chain CDR1, CDR2, and CDR3 sequences including SEQ ID NOs: 247-249, respectively;

(47) the heavy chain CDR1, CDR2, and CDR3 sequences including SEQ ID NOs: 207-209, respectively, and/or the light chain CDR1, CDR2, and CDR3 sequences including SEQ ID NOs: 252-254, respectively;

(48) the heavy chain CDR1, CDR2, and CDR3 sequences including SEQ ID NOs: 207-209, respectively, and/or the light chain CDR1, CDR2, and CDR3 sequences including SEQ ID NOs: 257-259, respectively;

(49) the heavy chain CDR1, CDR2, and CDR3 sequences including SEQ ID NOs: 212-214, respectively, and/or the light chain CDR1, CDR2, and CDR3 sequences including SEQ ID NOs: 262-264, respectively;

(50) the heavy chain CDR1, CDR2, and CDR3 sequences including SEQ ID NOs: 212-214, respectively, and/or the light chain CDR1, CDR2, and CDR3 sequences including SEQ ID NOs: 267-269, respectively;

(51) the heavy chain CDR1, CDR2, and CDR3 sequences including SEQ ID NOs: 217-219, respectively, and/or the light chain CDR1, CDR2, and CDR3 sequences including SEQ ID NOs: 262-264, respectively;

(52) the heavy chain CDR1, CDR2, and CDR3 sequences including SEQ ID NOs: 217-219, respectively, and/or the light chain CDR1, CDR2, and CDR3 sequences including SEQ ID NOs: 267-269, respectively;

(53) the heavy chain CDR1, CDR2, and CDR3 sequences including SEQ ID NOs: 222-224, respectively, and/or the light chain CDR1, CDR2, and CDR3 sequences including SEQ ID NOs: 262-264, respectively;

(54) the heavy chain CDR1, CDR2, and CDR3 sequences including SEQ ID NOs: 222-224, respectively, and/or the light chain CDR1, CDR2, and CDR3 sequences including SEQ ID NOs: 267-269, respectively;

(55) the heavy chain CDR1, CDR2, and CDR3 sequences including SEQ ID NOs: 227-229, respectively, and/or the light chain CDR1, CDR2, and CDR3 sequences including SEQ ID NOs: 262-264, respectively;

(56) the heavy chain CDR1, CDR2, and CDR3 sequences including SEQ ID NOs: 227-229, respectively, and/or the light chain CDR1, CDR2, and CDR3 sequences including SEQ ID NOs: 267-269, respectively;

(57) the heavy chain CDR1, CDR2, and CDR3 sequences including SEQ ID NOs: 232-234, respectively, and/or the light chain CDR1, CDR2, and CDR3 sequences including SEQ ID NOs: 262-264, respectively;

(58) the heavy chain CDR1, CDR2, and CDR3 sequences including SEQ ID NOs: 232-234, respectively, and/or the light chain CDR1, CDR2, and CDR3 sequences including SEQ ID NOs: 267-269, respectively.

The antibody or antigen-binding portion thereof according to the preceding aspect comprises a heavy chain variable region selected from the amino acid sequence of SEQ ID NOs: 1, 11, 21, 31, 41, 51, 61, 71, 81, 91, 101, 111, 121, 131, 141, 151, 161, 171, 181, 186, 191, 196, 201, 206, 211, 216, 221, 226, 231, or any variant thereof, and/or a light chain variable region selected from the amino acid sequence of SEQ ID NOs: 6, 16, 26, 36, 46, 56, 66, 76, 86, 96, 106, 116, 126, 136, 146, 156, 166, 176, 236, 241, 246, 251, 256, 261, 266, or any variant thereof.

In one aspect, the present disclosure relates to an antibody or antigen-binding portion thereof that binds human CD38, comprising a heavy chain variable region of the amino acid sequence of SEQ ID NO: 1 or any variant thereof, and a light chain variable region of the amino acid sequence of SEQ ID NO: 6 or any variant thereof.

In one aspect, the present disclosure relates to an antibody or antigen-binding portion thereof that binds human CD38, comprising a heavy chain variable region of the amino acid sequence of SEQ ID NO: 11 or any variant thereof, and a light chain variable region of the amino acid sequence of SEQ ID NO: 16 or any variant thereof.

In one aspect, the present disclosure relates to an antibody or antigen-binding portion thereof that binds human CD38, comprising a heavy chain variable region of the amino acid sequence of SEQ ID NO: 21 or any variant thereof, and a light chain variable region of the amino acid sequence of SEQ ID NO: 26 or any variant thereof.

In one aspect, the present disclosure relates to an antibody or antigen-binding portion thereof that binds human CD38, comprising a heavy chain variable region of the amino acid sequence of SEQ ID NO: 31 or any variant thereof, and a light chain variable region of the amino acid sequence of SEQ ID NO: 36 or any variant thereof.

In one aspect, the present disclosure relates to an antibody or antigen-binding portion thereof that binds human CD38, comprising a heavy chain variable region of the amino acid sequence of SEQ ID NO: 41 or any variant thereof, and a light chain variable region of the amino acid sequence of SEQ ID NO: 46 or any variant thereof.

In one aspect, the present disclosure relates to an antibody or antigen-binding portion thereof that binds human CD38, comprising a heavy chain variable region of the amino acid sequence of SEQ ID NO: 51 or any variant thereof, and a light chain variable region of the amino acid sequence of SEQ ID NO: 56 or any variant thereof.

In one aspect, the present disclosure relates to an antibody or antigen-binding portion thereof that binds human CD38, comprising a heavy chain variable region of the amino acid sequence of SEQ ID NO: 61 or any variant thereof, and a light chain variable region of the amino acid sequence of SEQ ID NO: 66 or any variant thereof.

In one aspect, the present disclosure relates to an antibody or antigen-binding portion thereof that binds human CD38, comprising a heavy chain variable region of the amino acid sequence of SEQ ID NO: 71 or any variant thereof, and a light chain variable region of the amino acid sequence of SEQ ID NO: 76 or any variant thereof.

In one aspect, the present disclosure relates to an antibody or antigen-binding portion thereof that binds human CD38, comprising a heavy chain variable region of the amino acid sequence of SEQ ID NO: 81 or any variant thereof, and a light chain variable region of the amino acid sequence of SEQ ID NO: 86 or any variant thereof.

In one aspect, the present disclosure relates to an antibody or antigen-binding portion thereof that binds human CD38, comprising a heavy chain variable region of the amino acid sequence of SEQ ID NO: 91 or any variant thereof, and a light chain variable region of the amino acid sequence of SEQ ID NO: 96 or any variant thereof.

In one aspect, the present disclosure relates to an antibody or antigen-binding portion thereof that binds human CD38, comprising a heavy chain variable region of the amino acid sequence of SEQ ID NO: 101 or any variant thereof, and a light chain variable region of the amino acid sequence of SEQ ID NO: 106 or any variant thereof.

In one aspect, the present disclosure relates to an antibody or antigen-binding portion thereof that binds human CD38, comprising a heavy chain variable region of the amino acid sequence of SEQ ID NO: 111 or any variant thereof, and a light chain variable region of the amino acid sequence of SEQ ID NO: 116 or any variant thereof.

In one aspect, the present disclosure relates to an antibody or antigen-binding portion thereof that binds human CD38, comprising a heavy chain variable region of the amino acid sequence of SEQ ID NO: 121 or any variant thereof, and a light chain variable region of the amino acid sequence of SEQ ID NO: 126 or any variant thereof.

In one aspect, the present disclosure relates to an antibody or antigen-binding portion thereof that binds human CD38, comprising a heavy chain variable region of the amino acid sequence of SEQ ID NO: 131 or any variant thereof, and a light chain variable region of the amino acid sequence of SEQ ID NO: 136 or any variant thereof.

In one aspect, the present disclosure relates to an antibody or antigen-binding portion thereof that binds human CD38, comprising a heavy chain variable region of the amino acid sequence of SEQ ID NO: 141 or any variant thereof, and a light chain variable region of the amino acid sequence of SEQ ID NO: 146 or any variant thereof.

In one aspect, the present disclosure relates to an antibody or antigen-binding portion thereof that binds human CD38, comprising a heavy chain variable region of the amino acid sequence of SEQ ID NO: 151 or any variant thereof, and a light chain variable region of the amino acid sequence of SEQ ID NO: 156 or any variant thereof.

In one aspect, the present disclosure relates to an antibody or antigen-binding portion thereof that binds human CD38, comprising a heavy chain variable region of the amino acid sequence of SEQ ID NO: 161 or any variant thereof, and a light chain variable region of the amino acid sequence of SEQ ID NO: 166 or any variant thereof.

In one aspect, the present disclosure relates to an antibody or antigen-binding portion thereof that binds human CD38, comprising a heavy chain variable region of the amino acid sequence of SEQ ID NO: 171 or any variant thereof, and a light chain variable region of the amino acid sequence of SEQ ID NO: 176 or any variant thereof.

In one aspect, the present disclosure relates to an antibody or antigen-binding portion thereof that binds human CD38, comprising a heavy chain variable region of the amino acid sequence of SEQ ID NO: 181 or any variant thereof, and a light chain variable region of the amino acid sequence of SEQ ID NO: 236 or any variant thereof.

In one aspect, the present disclosure relates to an antibody or antigen-binding portion thereof that binds human CD38, comprising a heavy chain variable region of the amino acid sequence of SEQ ID NO: 181 or any variant thereof, and a light chain variable region of the amino acid sequence of SEQ ID NO: 241 or any variant thereof.

In one aspect, the present disclosure relates to an antibody or antigen-binding portion thereof that binds human CD38, comprising a heavy chain variable region of the amino acid sequence of SEQ ID NO: 181 or any variant thereof, and a light chain variable region of the amino acid sequence of SEQ ID NO: 246 or any variant thereof.

In one aspect, the present disclosure relates to an antibody or antigen-binding portion thereof that binds human CD38, comprising a heavy chain variable region of the amino acid sequence of SEQ ID NO: 181 or any variant thereof, and a light chain variable region of the amino acid sequence of SEQ ID NO: 251 or any variant thereof.

In one aspect, the present disclosure relates to an antibody or antigen-binding portion thereof that binds human CD38, comprising a heavy chain variable region of the amino acid sequence of SEQ ID NO: 181 or any variant thereof, and a light chain variable region of the amino acid sequence of SEQ ID NO: 256 or any variant thereof.

In one aspect, the present disclosure relates to an antibody or antigen-binding portion thereof that binds human CD38, comprising a heavy chain variable region of the amino acid sequence of SEQ ID NO: 186 or any variant thereof, and a light chain variable region of the amino acid sequence of SEQ ID NO: 236 or any variant thereof.

In one aspect, the present disclosure relates to an antibody or antigen-binding portion thereof that binds human CD38, comprising a heavy chain variable region of the amino acid sequence of SEQ ID NO: 186 or any variant thereof, and a light chain variable region of the amino acid sequence of SEQ ID NO: 241 or any variant thereof.

In one aspect, the present disclosure relates to an antibody or antigen-binding portion thereof that binds human CD38, comprising a heavy chain variable region of the amino acid sequence of SEQ ID NO: 186 or any variant thereof, and a light chain variable region of the amino acid sequence of SEQ ID NO: 246 or any variant thereof.

In one aspect, the present disclosure relates to an antibody or antigen-binding portion thereof that binds human CD38, comprising a heavy chain variable region of the amino acid sequence of SEQ ID NO: 186 or any variant thereof, and a light chain variable region of the amino acid sequence of SEQ ID NO: 251 or any variant thereof.

In one aspect, the present disclosure relates to an antibody or antigen-binding portion thereof that binds human CD38, comprising a heavy chain variable region of the amino acid sequence of SEQ ID NO: 186 or any variant thereof, and a light chain variable region of the amino acid sequence of SEQ ID NO: 256 or any variant thereof.

In one aspect, the present disclosure relates to an antibody or antigen-binding portion thereof that binds human CD38, comprising a heavy chain variable region of the amino acid sequence of SEQ ID NO: 191 or any variant thereof, and a light chain variable region of the amino acid sequence of SEQ ID NO: 236 or any variant thereof.

In one aspect, the present disclosure relates to an antibody or antigen-binding portion thereof that binds human CD38, comprising a heavy chain variable region of the amino acid sequence of SEQ ID NO: 191 or any variant thereof, and a light chain variable region of the amino acid sequence of SEQ ID NO: 241 or any variant thereof.

In one aspect, the present disclosure relates to an antibody or antigen-binding portion thereof that binds human CD38, comprising a heavy chain variable region of the amino acid sequence of SEQ ID NO: 191 or any variant thereof, and a light chain variable region of the amino acid sequence of SEQ ID NO: 246 or any variant thereof.

In one aspect, the present disclosure relates to an antibody or antigen-binding portion thereof that binds human CD38, comprising a heavy chain variable region of the amino acid sequence of SEQ ID NO: 191 or any variant thereof, and a light chain variable region of the amino acid sequence of SEQ ID NO: 251 or any variant thereof.

In one aspect, the present disclosure relates to an antibody or antigen-binding portion thereof that binds human CD38, comprising a heavy chain variable region of the amino acid sequence of SEQ ID NO: 191 or any variant thereof, and a light chain variable region of the amino acid sequence of SEQ ID NO: 256 or any variant thereof.

In one aspect, the present disclosure relates to an antibody or antigen-binding portion thereof that binds human CD38, comprising a heavy chain variable region of the amino acid sequence of SEQ ID NO: 196 or any variant thereof, and a light chain variable region of the amino acid sequence of SEQ ID NO: 236 or any variant thereof.

In one aspect, the present disclosure relates to an antibody or antigen-binding portion thereof that binds human CD38, comprising a heavy chain variable region of the amino acid sequence of SEQ ID NO: 196 or any variant thereof, and a light chain variable region of the amino acid sequence of SEQ ID NO: 241 or any variant thereof.

In one aspect, the present disclosure relates to an antibody or antigen-binding portion thereof that binds human CD38, comprising a heavy chain variable region of the amino acid sequence of SEQ ID NO: 196 or any variant thereof, and a light chain variable region of the amino acid sequence of SEQ ID NO: 246 or any variant thereof.

In one aspect, the present disclosure relates to an antibody or antigen-binding portion thereof that binds human CD38, comprising a heavy chain variable region of the amino acid sequence of SEQ ID NO: 196 or any variant thereof, and a light chain variable region of the amino acid sequence of SEQ ID NO: 251 or any variant thereof.

In one aspect, the present disclosure relates to an antibody or antigen-binding portion thereof that binds human CD38, comprising a heavy chain variable region of the amino acid sequence of SEQ ID NO: 196 or any variant thereof, and a light chain variable region of the amino acid sequence of SEQ ID NO: 256 or any variant thereof.

In one aspect, the present disclosure relates to an antibody or antigen-binding portion thereof that binds human CD38, comprising a heavy chain variable region of the amino acid sequence of SEQ ID NO: 201 or any variant thereof, and a light chain variable region of the amino acid sequence of SEQ ID NO: 236 or any variant thereof.

In one aspect, the present disclosure relates to an antibody or antigen-binding portion thereof that binds human CD38, comprising a heavy chain variable region of the amino acid sequence of SEQ ID NO: 201 or any variant thereof, and a light chain variable region of the amino acid sequence of SEQ ID NO: 241 or any variant thereof.

In one aspect, the present disclosure relates to an antibody or antigen-binding portion thereof that binds human CD38, comprising a heavy chain variable region of the amino acid sequence of SEQ ID NO: 201 or any variant thereof, and a light chain variable region of the amino acid sequence of SEQ ID NO: 246 or any variant thereof.

In one aspect, the present disclosure relates to an antibody or antigen-binding portion thereof that binds human CD38, comprising a heavy chain variable region of the amino acid sequence of SEQ ID NO: 201 or any variant thereof, and a light chain variable region of the amino acid sequence of SEQ ID NO: 251 or any variant thereof.

In one aspect, the present disclosure relates to an antibody or antigen-binding portion thereof that binds human CD38, comprising a heavy chain variable region of the amino acid sequence of SEQ ID NO: 201 or any variant thereof, and a light chain variable region of the amino acid sequence of SEQ ID NO: 256 or any variant thereof.

In one aspect, the present disclosure relates to an antibody or antigen-binding portion thereof that binds human CD38, comprising a heavy chain variable region of the amino acid sequence of SEQ ID NO: 206 or any variant thereof, and a light chain variable region of the amino acid sequence of SEQ ID NO: 236 or any variant thereof.

In one aspect, the present disclosure relates to an antibody or antigen-binding portion thereof that binds human CD38, comprising a heavy chain variable region of the amino acid sequence of SEQ ID NO: 206 or any variant thereof, and a light chain variable region of the amino acid sequence of SEQ ID NO: 241 or any variant thereof.

In one aspect, the present disclosure relates to an antibody or antigen-binding portion thereof that binds human CD38, comprising a heavy chain variable region of the amino acid sequence of SEQ ID NO: 206 or any variant thereof, and a light chain variable region of the amino acid sequence of SEQ ID NO: 246 or any variant thereof.

In one aspect, the present disclosure relates to an antibody or antigen-binding portion thereof that binds human CD38, comprising a heavy chain variable region of the amino acid sequence of SEQ ID NO: 206 or any variant thereof, and a light chain variable region of the amino acid sequence of SEQ ID NO: 251 or any variant thereof.

In one aspect, the present disclosure relates to an antibody or antigen-binding portion thereof that binds human CD38, comprising a heavy chain variable region of the amino acid sequence of SEQ ID NO: 206 or any variant thereof, and a light chain variable region of the amino acid sequence of SEQ ID NO: 256 or any variant thereof.

In one aspect, the present disclosure relates to an antibody or antigen-binding portion thereof that binds human CD38, comprising a heavy chain variable region of the amino acid sequence of SEQ ID NO: 211 or any variant thereof, and a light chain variable region of the amino acid sequence of SEQ ID NO: 261 or any variant thereof.

In one aspect, the present disclosure relates to an antibody or antigen-binding portion thereof that binds human CD38, comprising a heavy chain variable region of the amino acid sequence of SEQ ID NO: 211 or any variant thereof, and a light chain variable region of the amino acid sequence of SEQ ID NO: 266 or any variant thereof.

In one aspect, the present disclosure relates to an antibody or antigen-binding portion thereof that binds human CD38, comprising a heavy chain variable region of the amino acid sequence of SEQ ID NO: 216 or any variant thereof, and a light chain variable region of the amino acid sequence of SEQ ID NO: 261 or any variant thereof.

In one aspect, the present disclosure relates to an antibody or antigen-binding portion thereof that binds human CD38, comprising a heavy chain variable region of the amino acid sequence of SEQ ID NO: 216 or any variant thereof, and a light chain variable region of the amino acid sequence of SEQ ID NO: 266 or any variant thereof.

In one aspect, the present disclosure relates to an antibody or antigen-binding portion thereof that binds human CD38, comprising a heavy chain variable region of the amino acid sequence of SEQ ID NO: 221 or any variant thereof, and a light chain variable region of the amino acid sequence of SEQ ID NO: 261 or

In one aspect, the present disclosure relates to an antibody or antigen-binding portion thereof that binds human CD38, comprising a heavy chain variable region of the amino acid sequence of SEQ ID NO: 221 or any variant thereof, and a light chain variable region of the amino acid sequence of SEQ ID NO: 266 or any variant thereof.

In one aspect, the present disclosure relates to an antibody or antigen-binding portion thereof that binds human CD38, comprising a heavy chain variable region of the amino acid sequence of SEQ ID NO: 226 or any variant thereof, and a light chain variable region of the amino acid sequence of SEQ ID NO: 261 or any variant thereof.

In one aspect, the present disclosure relates to an antibody or antigen-binding portion thereof that binds human CD38, comprising a heavy chain variable region of the amino acid sequence of SEQ ID NO: 226 or any variant thereof, and a light chain variable region of the amino acid sequence of SEQ ID NO: 266 or any variant thereof.

In one aspect, the present disclosure relates to an antibody or antigen-binding portion thereof that binds human CD38, comprising a heavy chain variable region of the amino acid sequence of SEQ ID NO: 231 or any variant thereof, and a light chain variable region of the amino acid sequence of SEQ ID NO: 261 or any variant thereof.

In one aspect, the present disclosure relates to an antibody or antigen-binding portion thereof that binds human CD38, comprising a heavy chain variable region of the amino acid sequence of SEQ ID NO: 231 or any variant thereof, and a light chain variable region of the amino acid sequence of SEQ ID NO: 266 or any variant thereof.

In one aspect, the present disclosure provides a bispecific or multispecific molecule comprising an antibody or antigen-binding portion thereof of any of the preceding aspects.

A nucleic acid molecule encodes the antibody or antigen-binding portion thereof or bispecific or multispecific molecule according to any of the preceding aspects. Preferably, the nucleic acid molecule comprises the nucleotide sequence of the antibody heavy chain selected from SEQ ID NOs: 5, 15, 25, 35, 45, 55, 65, 75, 85, 95, 105, 115, 125, 135, 145, 155, 165, 175, 185, 190, 195, 200, 205, 210, 215, 220, 225, 230, 235, or any variant thereof, and/or the nucleotide sequence of the antibody light chain selected from SEQ ID NOs: 10, 20, 30, 40, 50, 60, 70, 80, 90, 100, 110, 120, 130, 140, 150, 160, 170, 180, 240, 245, 250, 255, 260, 265, 270, or any variant thereof.

An antibody or antigen-binding portion thereof that binds to human CD38 has at least greater than 60%, 65%, 70%, 75%, 80%, 85%, 90%, 95%, 96%, 97%, 98%, 99%, or more sequence identity to the antibody or antigen-binding portion thereof of any of the preceding aspects.

A nucleic acid molecule encodes the antibody or antigen-binding portion thereof according to any one of the preceding aspects, or a nucleic acid molecule has at least greater than 60%, 65%, 70%, 75%, 80%, 85%, 90%, 95%, 96%, 97%, 98%, 99%, or more sequence identity thereto.

A vector comprises the nucleic acid according to any one of the preceding aspects.

A cell comprises the vector according to any one of the preceding aspects.

A pharmaceutical composition comprises the antibody or antigen-binding portion thereof or nucleic acid encoding the same according to any one of the preceding aspects and a pharmaceutically acceptable carrier. The antibodies of the present invention are useful as therapeutic or diagnostic tools in a variety of diseases in which CD38 is undesirably expressed or found. Diseases and conditions that are particularly suitable for the treatment with the CD38 antibodies of the present invention are tumors or autoimmune diseases, preferably said tumors are selected from the group consisting of multiple myeloma (MM) and chronic lymphocytic leukemia (CLL), chronic myelogenous leukemia (CML), T-cell acute lymphocytic leukemia, acute myelogenous leukemia (AML), and acute lymphocytic leukemia (ALL). Preferably, the autoimmune disease is selected from rheumatoid arthritis (RA) or systemic lupus erythematosus (SLE).

The method of treating diseases and conditions with the CD38 antibodies of the present invention includes the steps of administering to the mammal a therapeutically effective amount of an antibody or antigen-binding fragment thereof or a nucleic acid molecule or a vector or a cell or a pharmaceutical composition of any of the preceding aspects.

Use of the antibody or antigen-binding fragment thereof or the nucleic acid molecule or the vector or the cell or the pharmaceutical composition according to any of the preceding aspects in the manufacture of a medicament for the treatment of a CD38-related disorder in a mammal According to any one of the preceding aspects, optionally, the antibody is conjugated to other drugs, such as a labeled or cytotoxic conjugate.

In one aspect, the present disclosure also includes kits, e.g., comprising the antibodies, the fragments, the homologues, derivatives thereof, etc., of the disclosure, e.g., the labeled or cytotoxic conjugate, as well as the instructions for use of the antibody, the conjugate that kills a particular type of cell, and the like. The instructions can include directions for using the antibody, conjugate, etc. in vitro, in vivo, or ex vivo. The antibodies may be in liquid form or in solid form, usually lyophilized. The kit may contain other suitable reagents, such as buffers, reconstitution solutions and other necessary components for the intended use. Combinations of reagents packaged in predetermined amounts with instructions for their use, e. g. for therapeutic use or for conducting diagnostic assays, are contemplated. When the antibody is labeled, e. g. with an enzyme, then the kit can include a substrate and cofactors required for the enzyme (e. g. a substrate precursor providing a detectable chromophore or fluorophore). In addition, other additives such as stabilizers, buffers (e. g. blocking buffers or lysis buffers), and the like may also be included. The relative amounts of the various reagents can be varied to provide a concentrate of reagent solution, which provides user flexibility, and savings space and reagent. These reagents may also be provided in dry powder form, usually in lyophilized form, including excipients which, when dissolved, provide a reagent solution having the appropriate concentration.

Use of the antibody or functional fragment thereof or the nucleic acid molecule or the vector or the cell or the pharmaceutical composition or the kit according to any of the preceding aspects in the manufacture of a reagent for inhibiting the binding of CD38 to CD38R.

In addition, the antibodies of the present invention may be used in immunoassays, purification methods, and other methods using immunoglobulins or fragments thereof. Such uses are well known in the art. Accordingly, the present invention also provides compositions comprising anti-CD38 antibody of the present invention, or fragment thereof, conveniently in combination with a pharmaceutically acceptable carrier, diluent, or excipient, as is conventional in the art.

As used herein, the term “pharmaceutical composition” refers to formulations of various preparations. Formulations containing therapeutically effective amounts of multivalent antibodies are in sterile liquid solution, liquid suspension, or lyophilized form, optionally containing stabilizers or excipients. The antibodies of the present invention may be used as compositions for administration alone, or can be used in combination with other active agents.

It should be appreciated that therapeutic agents according to the described embodiments will be administered with suitable pharmaceutically acceptable carriers, excipients, and other agents incorporated into formulations to provide improved transfer, delivery, tolerability, and the like. A large number of suitable formulations can be found in all pharmacopoeias known to pharmaceutical chemists: Remington's Pharmaceutical Sciences (15th ed. Mack Publishing Company, Easton, Pa. (1975)), particularly Chapter 87: Blaug, Seymour. These formulations include, for example, powders, pastes, ointments, gels, waxes, oils, lipids, lipid-containing (cationic or anionic) carriers (e. g., Lipofectin™), DNA conjugates, anhydrous syrups, oil-in-water and water-in-oil emulsions, emulsion polyethylene glycols (polyethylene glycols of various molecular weights), semi-solid gels, and semi-solid mixtures containing polyethylene glycol. Any of the foregoing mixtures may be suitable for the treatment or therapy according to the present invention, provided that the active ingredient in the formulation is not inactivated by the formulation and that the formulation is physiologically compatible and tolerates the route of administration.

In one embodiment, the antibody can be used as a therapeutic agent. Such agents will generally be used to treat, alleviate, and/or prevent a disease or pathology associated with aberrant expression, activity, and/or signaling of CD38 in a subject. The therapeutic regimens may be implemented using standard methods by identifying a subject, e. g., a human patient, having (or at risk of or developing) a disease or disorder associated with aberrant expression, activity, and/or signaling of CD38, e. g., a CD38-related disorder. An antibody preparation, preferably one with high specificity and affinity for its target antigen, is administered to a subject, which will generally have an effect due to its binding to the target. The administered antibody can eliminate or inhibit or interfere with expression, activity, and/or signaling function of the target (e. g., CD38). The administered antibody can eliminate or inhibit or interfere with the binding of the target (e. g., CD38) to the endogenous ligand to which it naturally binds. For example, an antibody binds to a target and modulates, blocks, inhibits, reduces, antagonizes, neutralizes, and/or otherwise interferes with the expression, activity, and/or signaling of CD38. In some embodiments, to treat a disease or disorder associated with aberrant expression of CD38, an antibody having heavy and light chain CDR can be administered to a subject.

In another embodiment, antibodies against CD38 can be used in methods known in the art relating to localization and/or quantitation of CD38 (e. g., for determining CD38 and/or levels of CD38 in an appropriate physiological sample, for diagnostic methods, for protein imaging, etc.). In a given embodiment, an antibody comprising an antibody-derived antigen-binding domain specific for CD38 or a derivative, fragment, analog or homolog thereof is used as a pharmaceutically active compound (hereinafter “therapeutic agent”).

In another embodiment, CD38 polypeptides can be isolated using antibodies specific for CD38 by standard techniques such as immunoaffinity, chromatography, or immunoprecipitation. The protein in a biological sample can be detected with antibodies (or fragments thereof) against the CD38 protein. In some embodiments, CD38 can be detected in a biological sample as part of a clinical testing procedure, for example, to determine the efficacy of a given therapeutic regimen. Detection may be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazine aminofluorescein, dansyl chloride, or phycoerythrin; one example of a luminescent material includes luminol; examples of bioluminescent materials include luciferase, luciferin and aequorin, and examples of suitable radioactive materials include ¹²⁵I, ¹³¹I, ³⁵S, or ³H.

In another embodiment, antibodies according to the present disclosure can be used as reagents for detecting the presence of CD38 or a protein fragment thereof in a sample. In some embodiments, the antibody comprises a detectable label. The antibody is a polyclonal antibody, or more preferably a monoclonal antibody. Intact antibodies or fragments thereof (e. g., Fab, scFv or F(ab′)₂) are used. The term “labeled” with respect to an antibody is intended to encompass direct labeling of the antibody by coupling (i. e., physically linking) a detectable substance to the antibody, as well as indirect labeling of the antibody by reaction with another reagent that is directly labeled. Examples of indirect labeling include detection of a primary antibody using a fluorescently labeled second antibody, and end-labeling of the antibody with biotin to enable detection with fluorescently labeled streptavidin. The term “biological sample” is intended to include tissues, cells, and biological fluids isolated from a subject, as well as tissues, cells, and fluids present in a subject. Thus, the term “biological sample” as used includes blood and fractions or components in blood, including serum, plasma, or lymph. In other words, the detection method of the described embodiments can be used to detect the analyte mRNA, protein, or genomic DNA in a biological sample both in vitro and in vivo. For example, in vitro techniques for detection of mRNA analytes include Norhtem hybridization and in situ hybridization. In vitro techniques for detection of protein analytes include enzyme-linked immunosorbent assays (ELISA), Western blot, immunoprecipitations, and immunofluorescence. In vitro techniques for detection of genomic DNA analytes include Southern hybridization. Procedures for performing immunoassays are described, for example, in “ELISA: Theory and Practice: Methods in Molecular Biology”, vol. 42, J. R. Crowther (ed.) Human Press, Totowa, N.J. 1995; “Immunoassay”, E. Diamandis and T. Christopoulus, Academic Press, Inc., San Diego, Calif., 1996; and “Practice and Theory of Enzyme Immunoassays”, P. Tijssen, Elsevier Science Publishers, Amsterdam, 1985. In addition, in vivo techniques for detection of protein analytes include introducing into a subject a labeled anti-analyte protein antibody. For example, an antibody can be labeled with a radiolabel, and the presence and location of the radiolabel in the subject can then be detected by standard imaging techniques. The antibodies described herein and derivatives, fragments, analogs, and homologs thereof can be incorporated into pharmaceutical compositions suitable for administration. The principles and considerations involved in preparing such compositions, as well as guidance in selecting components, are well known in the art, for example, see Remington's Pharmaceutical Sciences: The Science and Practice of Pharmacy, 19th edition (Alfonso R. Gennaro et al. Eds.) Mack Pub. Co. Easton, Pa.: 1995; Drug Absorption Enhancement: Concepts, Possibilities, Limitations, and Trends, Harwood Academic Publishers, Langhorne, Pa. 1994; and Peptide and Protein Drug Delivery (Advances In Parenteral Sciences, vol. 4), 1991, M. Dekker, New York.

Such compositions typically comprise the antibody and a pharmaceutically acceptable carrier. When antibody fragments are used, minimal inhibitory fragments that specifically bind to the target protein binding domain may be preferred. For example, based on the variable region sequence of an antibody, peptide molecules can be designed that retain the ability to bind to a target protein sequence. Such peptides can be chemically synthesized and/or produced by recombinant DNA techniques (see, e. g., Marasco et al. Proc. Natl. Acad. Sci. USA, 90:7889-7893 (1993)).

As used herein, the term “pharmaceutically acceptable carrier” is intended to include any and all solvents, dispersion media, coatings, antibacterial and antifungal agents, isotonic and absorption delaying agents, etc., compatible with pharmaceutical administration. Suitable pharmaceutically acceptable carriers are described in the latest edition of Remington's Pharmaceutical Sciences, a standard bibliography in the art, which is incorporated herein by reference. Preferred examples of such carriers or diluents include, but not limited to, water, saline, Ringer's solution, dextrose solution, and 5% human serum albumin. Liposomes and non-aqueous vehicles such as fixed oils can also be used. The use of such media and agents for pharmaceutically active substances is well known in the art. Except insofar as any conventional media or agent is incompatible with the antibody, its use in the compositions is contemplated.

The pharmaceutical compositions of the embodiments are formulated to be compatible with their intended route of administration. Examples of routes of administration include parenteral, e. g., intravenous, intradermal, subcutaneous, oral (e.g., inhalation), transdermal (i. e., topical), transmucosal, and rectal administration. Solutions or suspensions for parenteral, intradermal or subcutaneous administration may include the following components: sterile diluents for injection such as water, saline solutions, fixed oils, polyethylene glycols, glycerol, propylene glycol or other synthetic solvents; antibacterial agents such as benzyl alcohol or methyl p-hydroxybenzoate; antioxidants such as ascorbic acid or sodium bisulfite; chelating agents such as EDTA; buffers, such as acetates, citrates, or phosphates, and reagents to adjust the osmotic pressure, such as sodium chloride or dextrose. The pH can be adjusted with acids or bases, such as hydrochloric acid or sodium hydroxide. The parenteral preparation can be packaged in ampoules, disposable syringes, or multiple dose vials made of glass or plastic.

Pharmaceutical compositions suitable for injectable use include sterile aqueous solutions (water soluble) or dispersions and sterile powders for the extemporaneous preparation of sterile injectable solutions or dispersion. For intravenous administration, suitable pharmaceutically acceptable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating from microorganisms such as bacteria and fungi. The carrier can be a solvent or dispersion medium containing, for example, water, ethanol, polyol (for example, glycerol, propylene glycol, and liquid polyethylene glycol, etc.), and suitable mixtures thereof. For example, the desired particle size can be maintained in the case of dispersions by the use of coatings such as lecithin, and proper fluidity can be maintained by the use of surfactants. Prevention of the action of microorganisms can be achieved by various antibacterial and antifungal agents, for example, parabens, chlorobutanol, phenol, ascorbic acid, thimerosal, etc. In many cases, it will be preferable to include isotonic agents, for example, sugars, polyalcohols such as mannitol, sorbitol, and sodium chloride in the composition. The absorption of the injectable compositions can be prolonged by encompassing in the composition an agent which delays absorption, for example, aluminum monostearate and gelatin.

Sterile injectable solutions can be prepared by incorporating the antibody in the required amount in an appropriate solvent with one or a combination of ingredients enumerated above, as required, followed by filtered sterilization. Generally, dispersions are prepared by incorporating the antibody into a sterile vehicle that contains a basic dispersion medium and the required other ingredients from those enumerated above. The resulting powders are vacuum-dried and freeze-dried to prepare sterile powder for injection containing the active ingredient and any additional desired ingredient from a sterile-filtered solution of those ingredients previously described.

For administration by inhalation, the compounds are delivered in the form of an aerosol spray from a pressurized container or dispenser or a nebulizer containing a suitable propellant, such as carbon dioxide and other gas.

Systemic administration can also be performed by transmucosal or transdermal means. For transmucosal or transdermal administration, penetrants appropriate to permeate the barrier are used in the formulation. Such penetrants are generally known in the art, and include, for example, detergents, bile salts, and fusidic acid derivatives for transmucosal administration. Transmucosal administration can be accomplished through the use of nasal sprays or suppositories. For transdermal administration, one or more of the antibodies can be formulated into ointments, salves, gels, or creams as generally known in the art. The compounds may also be prepared in the form of suppositories (e. g., with conventional suppository bases such as cocoa butter or other glycerides) or retention enemas for rectal delivery.

In one embodiment, the antibodies can be prepared with carriers that prevent their rapid elimination from the body, such as sustained/controlled release formulations, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparing such formulations will be apparent to those skilled in the art.

It is especially advantageous to formulate parenteral compositions in dosage unit form for ease of administration and uniformity of dosage. Dosage unit form, as used herein, refers to physically discrete units suited as unitary dosages for the subject to be treated; each unit contains a predetermined quantity of one or more of the antibodies calculated to produce the desired therapeutic effect in association with the required pharmaceutical carrier. The specification for the dosage unit forms of the described embodiments are dictated by and directly dependent on: the unique characteristics of antibodies and the specific therapeutic effects to be achieved, and the limitations inherent in the art of formulating such antibodies for treating individuals.

The pharmaceutical compositions can be placed in a container, pack, or dispenser together with instructions for administration.

The formulations described herein may also contain more than one of the antibodies, preferably those with complementary activities but without adversely affecting each other, depending on the particular situation to be treated. Alternatively or additionally, the composition may, for example, comprise an agent that enhances the function thereof, such as a cytotoxic agent, a cytokine, a chemotherapeutic agent, or a growth inhibitor. Such molecules are suitably present in combination in amounts effective for the intended purpose. For example, they may be present in combination in a kit or in combination for use.

In one embodiment, one or more of the antibodies can be administered in combination therapy, i. e., in combination with other agents, such as therapeutic agents, which can be used to treat pathological conditions or disorders, such as various forms of cancer, autoimmune disorders, and inflammatory diseases. The term “in combination” means herein that the agents are administered substantially synchronously, simultaneously or sequentially. If administered sequentially, the first of the two compounds is still preferably detected at an effective concentration at the treatment site when the second compound is initially administered. In one aspect, a “combination” can also include both an antibody of the present invention and another therapeutic agent in a kit.

For example, combination therapy can comprise coformulation and/or coadministration of one or more antibodies described herein with one or more additional therapeutic agents as described in more detail below, for example, one or more cytokine and growth factor inhibitors, immunosuppressants, anti-inflammatory agents, metabolic inhibitors, enzyme inhibitors, and/or cytotoxins or cytostatic agents. Such combination therapy may advantageously utilize lower dosages of the administered therapeutic agents, thus avoiding possible toxicities or complications associated with each monotherapy. For purposes of clarity and conciseness, features are described herein as part of the same or separate embodiments, however, it will be understood that the scope of the present invention may include some embodiments having a combination of all or some of the features described.

### EXAMPLE

### Example 1: Preparation of CD38 Recombinant Proteins

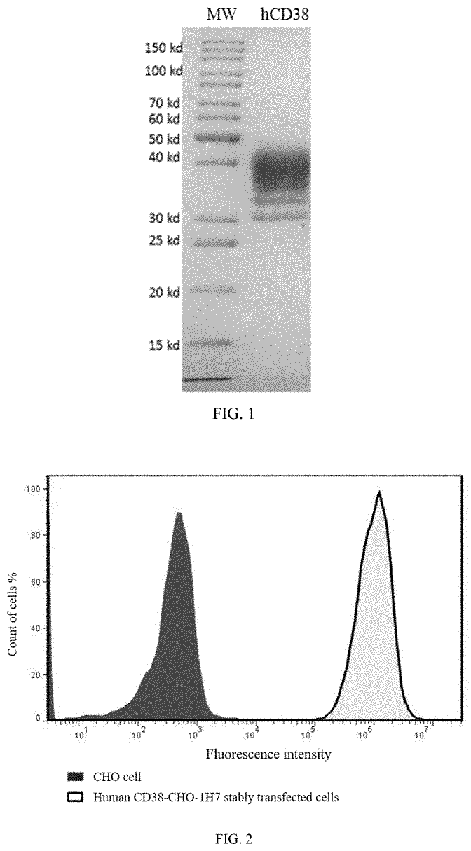

Human CD38 (UniProt KB: P28907) extracellular region (Val 43-Ile 300) nucleotide sequences were synthesized, to which IgG kappa signal peptide and 6×his tag were fused at N-terminal, the fused sequences were digested and cloned into eukaryotic expression plasmid CMV downstream of promoter to obtain a pSect-Nhis-CD38; HEK293.6E cells were transfected with the plasmid and cultured for 7 days, the supernatant of culture medium was collected and purified by nickel chelate column to obtain recombinant human CD38 extracellular region protein. See FIG. 1 for SDS-PAGE.

### Example 2. Construction of Human CD38/CHO Stably Transfected Cell Line

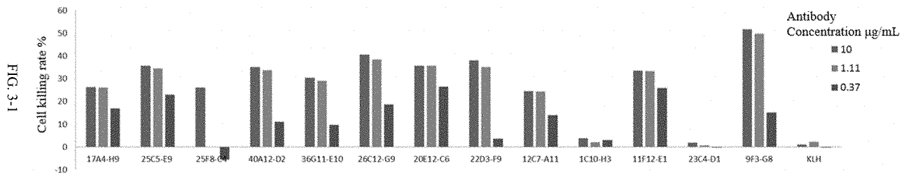

A lentiviral vector plasmid containing a full-length sequence of human CD38 was constructed, and the constructed lentiviral plasmid and packaging plasmid were co-transfected into HEK 293T cells for lentiviral packaging, according to the lentiviral packaging kit instructions (Lenti-Pac HIV Expression Packaging Kit, Gene Copoeia, Cat: HPK-LvTR-20). Culture medium was collected in 48 hours after transfection, and centrifuged at 500*g for 10 minutes to remove cell debris to obtain culture supernatant containing lentivirus particles, which was filtered through 0.45 μm PES membrane and dispensed into 1.5 mL EP tubes; 1×10⁶ CHO cells were infected with 10 μL filtrate, and 10 μg/mL puromycin was added to the culture medium for screening positive clones, which was limiting dilution to obtain stably transfected cells CHO-CD38-1H7 expressing human full-length CD38, with the results shown in FIG. 2.

### Example 3 Preparation of the Polyclonal Antibody Against Human CD38

1) Immunization of Mice

Human recombinant CD38 protein or CHO-CD38-1H7 cells were used as an antigen and mixed with an equal amount of immune adjuvant (Freund's adjuvant), and each group of 6 female Balb/c mice of 6 weeks were taken for subcutaneous immunization A booster immunization was performed two weeks after the primary immunization After three-time immunization, orbital bleeds were taken to detect serum titers.

2) Cell Fusion and Hybridoma Screening

Before fusion, 1×10⁶ CHO-CD38-1H7 transfected cells were injected into the tail vein challenging. Three days later, the mice were sacrificed by neck dissection. The mouse spleen and some peripheral lymph nodes were taken, centrifuged after milling in DMEM medium; after the supernatant was poured, the spleen cell mass was gently dispersed, and 5 ml of red blood cell lysis buffer was added. 40 ml DMEM was added after lysis for 50s to centrifuge, a spleen cell suspension without red blood cells was obtained. After mixing an appropriate amount of lymph node and the spleen cell suspension with SP2/0, the BTX electrofusion instrument was used for cell fusion. The fusion cells were seeded in a 96-well plate, and cultured in a complete DMEM medium containing HAT under 5% CO₂ condition at 37° C. The growth of hybridoma cells was observed in about a week, and the supernatant was taken out for detection when the hybridoma cells grew to more than 60%.

### Example 4. Screening of Monoclonal Antibodies Against Human CD38 and Acquisition of Antibody Sequences

1) ELISA Screening

Human CD38 recombinant protein was coated onto a 96-well microtiter plate at 1 μg/mL and incubated overnight at 4° C. The plate was washed for 3 times with PBS next day, blocked with 200 μL 2% skimmed milk powder/PBS for 2 hours at room temperature, and washed once with PBS, followed by the addition of 50 μL hybridoma supernatant, the mixture was incubated for 1 hour at room temperature, and repeatedly washed for 3 times with PBST and PBS, followed by the addition of 100 μL/well HRP labeled anti-mouse IgG Fc secondary antibody, the mixture was incubated for 60 minutes at room temperature. To the mixture washed for 3 times with PBST and PBS respectively was added 100 μL of chromogenic reagent (TMB solution, Sigma Cat: T2885), followed by standing at 37° C. for 5 minutes, the reaction was terminated by adding 50 μL of 2M concentrated sulfuric acid solution, and OD450 value was read immediately with a microplate reader.

2) Cell Screening Anti-CD38 Specific Antibodies (FACS)

Supernatants of positive clones that bound to CD38 antibodies detected by ELISA were further verified for binding to CD38 positive cells. CHO cells and CHO-CD38 stably transfected cells were taken out and added into a 96-well plate at 5×10⁴ cells/well, respectively, 50 μL hybridoma supernatant was added per well, the mixture was incubated at 4° C. for 60 minutes, then centrifuged to discard the supernatant by pipetting, sediment was washed with 0.5% BSA/PBS, 50 μL secondary antibody solution (anti-mouse IgG-Fc-AF647, Jackson ImmunoResearch, Cat: 115-606-071) was added, the mixture was incubated at 4° C. for 45 minutes, then washed off with 0.5% BSA/PBS to remove excess secondary antibody, the cells were resuspended by adding PBS, and detected by Flow cytometry.

3) Gene Cloning and Expression of Chimeric Anti-Human CD38 Antibody

Positive monoclonal hybridoma against CD38 was screened, total cell RNA was extracted by a TRNzol lysis method, then single-chain cDNA was synthesized using reverse transcription kit (Invitrogen, Cat. No. 18080051), and used as a template to amplify the variable region sequence of antibody in monoclonal hybridoma cells; after sequencing, the obtained candidate positive clone heavy chain and light chain variable region sequences were as follows respectively:

Clones: 21H12-G12
Heavy chain VH
SEQ ID NOs: 1-10
<-------------FR1-----------> CDR1 <----FR2-----> CDR2 <---
QIQLVQSGPELKKPGETVKISCKASGYTFTNHGMNWVKQAPGKGLKWMGWINTYTGEPTYGEDFKGRFA
--------------FR3-----------> CDR3<---FR4--->
FSLETSASTAYLQINNLKNEDLATYFCARKGFVYWGQGTLVTVSV
Nucleotide sequence
CAGATCCAGTTGGTGCAGTCTGGACCTGAGCTGAAGAAGCCTGGAGAGACAGTCAAGATCTCCTGCAAGGC
TTCTGGGTATACTTTCACAAACCATGGAATGAACTGGGTGAAGCAGGCTCCAGGAAAGGGCTTAAAGTGGA
TGGGCTGGATAAACACCTACACTGGAGAGCCAACATATGGTGAAGACTTCAAGGGACGGTTTGCCTTCTCT
TTGGAAACCTCTGCCAGCACTGCCTATTTGCAGATCAACAACCTCAAAAATGAGGACCTGGCTACATATTT
CTGTGCAAGAAAGGGGTTTGTTTACTGGGGCCAAGGGACTCTGGTCACTGTCTCTGTA
Light chain VK
<---------FR1---------> CDR1 <-----FR2----- > CDR2 <--------
DIVLTQSPASLAVSLGQRATMSCRASESVDIYGNSFIYWYQQKPGQPPKLLIYRASILESGIPARFSGS
---------FR3----------> CDR3 <---FR4--->
GSRTDFTLTINPVEADDVATYYCQQINEDPFTFGGGTKLEIK
Nucleotide SEQuence
GACATTGTGCTGACCCAATCTCCAGCTTCTTTGGCTGTGTCTCTAGGGCAGAGGGCCACCATGTCCTGCAG
AGCCAGTGAAAGTGTTGATATTTATGGCAATAGTTTTATATACTGGTACCAACAGAAACCAGGACAGCCAC
CCAAACTCCTCATCTATCGTGCATCCATCCTAGAATCTGGGATCCCTGCCAGGTTCAGTGGCAGTGGGTCT
AGGACAGACTTCACCCTCACCATTAATCCTGTGGAGGCTGATGATGTTGCAACCTATTACTGTCAGCAAAT
TAATGAAGATCCATTCACGTTCGGAGGGGGGACCAAGCTGGAAATAAAA
Clones: 7C10-G5
Heavy chain VH
SEQ ID NOs: 11-20
<-------------FR1-----------> CDR1 <----FR2-----> CDR2 <--
QAYLQQSGAELVRSGASVKMSCKASGYTFTSYNIHWVKQTPGQGLKWIGYIYPGNGGTNYNQKFKVKAT
-------------FR3-------------> CDR3 <---FR4--->
LTADTSSSTAYMQISSLTSEDSAVYFCARRGSDYDGGFASWGQGTLVTVSA
Nucleotide sequence
CAGGCTTATCTACAGCAGTCTGGGGCTGAGCTGGTGAGGTCTGGGGCCTCAGTGAAGATGTCCTGCAAGGC
TTCTGGCTACACATTTACCAGTTACAATATACACTGGGTAAAGCAGACACCTGGACAGGGCCTGAAATGGA
TTGGATATATCTATCCTGGAAATGGTGGTACTAACTACAATCAGAAGTTCAAGGTCAAGGCCACATTGACT
GCAGACACATCCTCCAGCACAGCCTACATGCAGATCAGCAGCCTGACATCTGAAGACTCTGCGGTCTATTT
CTGTGCAAGAAGGGGGTCTGATTACGACGGGGGGTTTGCTTCCTGGGGCCAAGGGACTCTGGTCACTGTCT
CTGCA
Light chain VK
<----------FR1--------> CDR1 <-----FR2-----> CDR2 <--------
DILLTQSPASLAVSLGQRATISCRASKSVDIYGNSFIHWYQQKPGQSPKLLIYLASNLDSGVPARFSGS
-----------FR3--------> CDR3 <---FR4--->
GSRTDFTLTIDPVEADDAATYYCQQNNEDPLTFGAGTKLELK
Nucleotide sequence
GACATTTTGCTGACCCAGTCTCCAGCTTCTTTGGCTGTGTCTCTAGGGCAGAGGGCCACCATATCCTGCAG
AGCCAGTAAAAGTGTTGATATTTATGGCAATAGTTTTATACACTGGTACCAGCAGAAACCAGGACAGTCAC
CCAAACTCCTCATCTATCTTGCATCCAACCTAGATTCTGGGGTCCCTGCCAGGTTCAGTGGCAGTGGGTCT
AGGACAGACTTCACCCTCACCATTGATCCTGTGGAGGCTGATGATGCTGCAACCTATTACTGTCAGCAAAA
TAATGAGGATCCGCTCACGTTCGGTGCTGGGACCAAGCTGGAGCTGAAA
Clones: 15A1-G1
Heavy chain VH
SEQ ID NOs: 21-30
<-------------FR1-----------> CDR1 <-----FR2----> CDR2 <---
QLQLKQSGPGLVQPSQSLSITCTVSGFSLTSFGVHWIRQSPGKGLEWLGVIWRGGSTDYNAAFMSRLSI
-------------FR3------------> CDR3 <---FR4--->
TKDNSKSQVFFKMNSLQADDTAIYYCAKTMITRGYTMDYWGQGTSVTVSS
Nucleotide sequence
CAGTTGCAGCTGAAGCAGTCAGGACCTGGCCTAGTGCAGCCCTCACAGAGCCTGTCCATAACCTGCACAGT
CTCTGGTTTCTCATTAACTAGCTTTGGTGTACACTGGATTCGCCAGTCTCCAGGAAAGGGTCTGGAGTGGC
TGGGAGTGATTTGGAGAGGTGGAAGCACAGACTACAATGCAGCTTTCATGTCCAGACTGAGCATCACCAAG
GACAACTCCAAGAGCCAAGTTTTCTTTAAAATGAACAGTCTGCAAGCTGATGACACTGCCATATATTACTG
TGCCAAAACTATGATTACGAGGGGGTATACTATGGACTACTGGGGTCAAGGAACCTCAGTCACCGTCTCCT
CA
Light chain VK
<----------FR1--------> CDR1 <------FR2----> CDR2 <------------
DIQMTQSSSSFSVSLGDRVTITCKASEDIYNRLAWYQQKPGNAPRLLISGATTLETGVPSRFSGSGSGK
--------FR3-------> CDR3 <---FR4--->
DYTLSITSLQTEDVATYYCQQFWSTPYTFGGGTKLEMI
Nucleotide sequence
GACATCCAGATGACACAATCCTCATCCTCCTTTTCTGTATCTCTAGGAGACAGGGTCACCATTACTTGCAA
GGCAAGTGAGGACATATATAATCGGTTAGCCTGGTATCAGCAGAAACCAGGAAATGCTCCTAGGCTCTTAA
TATCTGGTGCAACCACTTTGGAAACTGGGGTTCCTTCAAGATTCAGTGGCAGTGGATCTGGAAAGGATTAC
ACTCTCAGCATTACCAGTCTTCAGACTGAAGATGTTGCTACTTATTACTGTCAACAGTTTTGGAGTACTCC
GTACACGTTCGGAGGGGGGACCAAGCTGGAAATGATA
Clones: 51G7-D3
Heavy chain VH
SEQ ID NOs: 31-40
<------------FR1-------------> CDR1<-----FR2----> CDR2 <--
EVQLVESGGGLVKPGGSLKLSCEGSGFTFNNYAMSWVRQSPEKRLEWVAEISTGGTFTYYVDTVTGRFT
--------------FR3-----------> CDR3 <---FR4--->
ISRDNAKNTLYLEMSSLRSEDTARYYCARGYRYDAADYWGQGISVTVSS
Nucleotide sequence
GAAGTGCAGCTGGTGGAGTCTGGGGGAGGCTTAGTGAAGCCTGGAGGGTCCCTGAAACTCTCCTGTGAAGG
CTCTGGATTCACTTTCAACAACTATGCCATGTCTTGGGTTCGCCAGTCTCCAGAGAAGAGGCTGGAGTGGG
TCGCAGAGATTTCTACTGGTGGTACTTTCACCTACTATGTAGACACTGTGACGGGCCGATTCACCATCTCC
AGAGACAATGCCAAGAACACCCTGTACCTGGAAATGAGTAGTCTGAGGTCTGAGGACACGGCCAGGTATTA
TTGTGCAAGAGGCTATAGGTACGACGCGGCGGACTACTGGGGTCAAGGAATCTCAGTCACCGTCTCCTCA
Light chain VK
<----------FR1--------> CDR1 <-----FR2-----> CDR2 <------------
DIQMTQSPSSLSASLGERVSLTCRASQEISGYLIWLQQKPDGTIKRLIYATSTLDSGVPKRFSGSRSGS
--------FR3-------> CDR3 <---FR4--->
DYTLTISSLESEDFADYYCLQYANYFTFGAGTKLELK
Nucleotide sequence
GACATCCAGATGACCCAGTCCCCCTCCTCCCTCTCCGCCTCCCTCGGCGAGCGCGTCTCCCTCACCTGCCG
CGCCTCCCAGGAGATCTCCGGCTACCTCATCTGGCTCCAGCAGAAGCCCGACGGCACCATCAAGCGCCTCA
TCTACGCCACCTCCACCCTCGACTCCGGCGTCCCCAAGCGCTTCTCCGGCTCCCGCTCCGGCTCCGACTAC
ACCCTCACCATCTCCTCCCTCGAGTCCGAGGACTTCGCCGACTACTACTGCCTCCAGTACGCCAACTACTT
CACCTTCGGCGCCGGCACCAAGCTCGAGCTCAAG
Clones: 17A4-H9
Heavy chain VH
SEQ ID NOs: 41-50
<------------FR1-----------> CDR1 <-----FR2----> CDR2 <---
QVQLNQSGPGLVQPSQSLSITCTVSGFSLTSYGVHWLRQSAGKGLEWLGVIWRGGSTDYNVGFMSRLTI
-------------FR3-----------> CDR3 <---FR4--->
SKDNSKSQVFFKMNSLQGDDTAIYFCAKSMITTGYTMDYWGQGTSVTVSS
Nucleotide sequence
CAGGTGCAGCTGAACCAGTCAGGACCTGGCCTAGTGCAGCCCTCACAGAGCCTGTCCATAACCTGCACAGT
CTCTGGTTTCTCATTAACTAGCTATGGTGTACATTGGCTTCGCCAGTCTGCAGGAAAGGGTCTGGAGTGGC
TGGGAGTGATATGGAGAGGTGGAAGCACAGACTACAATGTAGGTTTCATGTCCAGACTGACCATTAGTAAG
GACAATTCCAAGAGCCAAGTTTTCTTTAAAATGAACAGTCTGCAAGGTGATGACACTGCCATATACTTCTG
TGCCAAAAGTATGATTACGACGGGCTATACTATGGACTACTGGGGTCAAGGAACCTCAGTCACCGTCTCCT
CA
Light chain VK
<----------FR1--------> CDR1 <-----FR2-----> CDR2 <------------
AIQMTQSSSSFSVSLGDRVTITCKASEDIYNRLAWYQQKSGNAPRLLISGSTSLETGVPSRFSGSGSGK
--------FR3-------> CDR3 <---FR4--->
DYTLSIISLQTEDVATYYCQQFWSTPWTFGGGTKLEIK
Nucleotide sequence
GCCATCCAGATGACACAATCTTCATCCTCCTTTTCTGTATCTCTAGGAGACAGAGTCACCATTACTTGCAA
GGCAAGTGAGGACATATATAATCGGTTAGCTTGGTATCAGCAGAAATCAGGAAATGCTCCAAGGCTCTTAA
TATCTGGTTCAACCAGTTTGGAAACTGGGGTTCCTTCAAGATTCAGTGGCAGTGGATCTGGAAAGGATTAC
ACTCTCAGCATTATCAGTCTTCAGACTGAAGATGTTGCTACTTATTACTGTCAACAGTTTTGGAGTACTCC
GTGGACGTTCGGTGGAGGCACCAAGCTGGAAATAAAA
Clones: 25C5-E9
Heavy chain VH
SEQ ID NOs: 51-60
<-------------FR1-----------> CDR1 <-----FR2----> CDR2 <---
QVQLKQSGPGLVQPSQSLSITCTVSGFSLTSYGVHWVRQSPGKGLEWLGVMWRGGSTDYNAPFMSRLSI
-------------FR3-----------> CDR3 <---FR4--->
TKDNSKSQVFFKMNSLQADDTAIYYCAKSLITTGYAMDYWGQGTSVTVSS
Nucleotide sequence
CAGGTCCAGCTGAAGCAGTCAGGACCTGGCCTAGTGCAGCCCTCACAGAGCCTGTCCATAACCTGCACAGT
CTCTGGTTTCTCATTAACTAGCTATGGTGTACACTGGGTTCGCCAGTCTCCAGGAAAGGGTCTGGAGTGGC
TGGGAGTGATGTGGAGAGGTGGAAGCACAGACTACAATGCACCTTTCATGTCCAGACTGAGCATCACCAAG
GACAACTCCAAGAGCCAAGTTTTCTTTAAAATGAACAGTCTGCAAGCTGATGACACTGCCATATACTACTG
TGCCAAAAGTCTGATTACGACTGGGTATGCTATGGACTACTGGGGTCAAGGAACCTCAGTCACCGTCTCCT
CA
Light chain VK
<----------FR1--------> CDR1 <-----FR2-----> CDR2 <-------------
DIQMTQSSSSFSVSLGDRVTITCKASEDIYNRLAWYQQKPGNAPRLLISGATSLETGVPSRFSGSGSGK
--------FR3-------> CDR3 <---FR4--->
DYTLSITSLQTEDVATYYCQQYWSNPYTFGGGTKLEIK
Nucleotide sequence
GACATCCAGATGACACAATCTTCATCCTCCTTTTCTGTATCTCTAGGAGACAGAGTCACCATTACTTGCAA
GGCAAGTGAGGACATATATAATCGGTTAGCCTGGTATCAGCAGAAACCAGGAAATGCTCCTAGGCTCTTAA
TATCTGGTGCAACCAGTTTGGAAACTGGGGTTCCTTCAAGATTCAGTGGCAGTGGATCTGGAAAGGATTAC
ACTCTCAGCATTACCAGTCTTCAGACTGAAGATGTTGCTACTTATTACTGTCAACAGTATTGGAGTAATCC
GTACACGTTCGGAGGGGGGACCAAGCTGGAAATAAAA
Clones: 25F8-C4
Heavy chain VH
SEQ ID NOs: 61-70
<-------------FR1------------> CDR1<-----FR2----> CDR2 <--
QAYLQQSGAELVRSGASVKMSCKASGYTFSSYNMHWVKQTPGQGLEWIGYVYPGNGGTNYNQKFKGKAT
-------------FR3------------> CDR3 <---FR4--->
LTADTSSSTAYMQISSLTSEESAVYFCARPESSFHPWFAYWGQGTLVTVAP
Nucleotide sequence
CAGGCTTATCTACAGCAGTCTGGGGCTGAGCTGGTGAGGTCTGGGGCCTCAGTGAAGATGTCCTGCAAGGC
TTCTGGCTACACATTTTCCAGTTACAATATGCACTGGGTAAAGCAGACACCTGGACAGGGCCTGGAATGGA
TTGGATATGTTTATCCTGGAAATGGTGGTACGAACTACAATCAGAAGTTCAAGGGCAAGGCCACATTGACT
GCAGACACATCCTCCAGCACAGCCTACATGCAGATCAGCAGCCTGACATCTGAAGAGTCTGCGGTCTATTT
CTGTGCAAGACCAGAGAGTAGTTTCCACCCCTGGTTTGCTTACTGGGGCCAAGGGACTCTGGTCACTGTCG
CGCCA
Light chain VK
<---------FR1---------> CDR1 <-----FR2-----> CDR2 <---------
NIVLTQSPASLAVSLGQRATISCRASESVNIYGNIFMYWYQQKPGQPPKLLIYLASNLASGVPARFSGS
-----------FR3--------> CDR3 <---FR4--->
GSRTGFTLTIDPVEADDAATYYCQQNAKDPWTFGGGTKLEIK
Nucleotide sequence
AACATTGTGCTGACCCAATCTCCAGCTTCTTTGGCTGTGTCTCTAGGGCAGAGGGCCACCATATCCTGCAG
AGCCAGTGAAAGTGTTAATATTTATGGCAATATTTTTATGTACTGGTACCAGCAGAAACCAGGACAGCCAC
CCAAACTCCTCATCTATCTTGCATCCAACCTAGCATCTGGGGTCCCTGCCAGGTTCAGTGGCAGTGGGTCT
AGGACAGGCTTCACCCTCACCATTGATCCTGTGGAGGCTGATGATGCTGCAACCTATTACTGTCAGCAAAA
TGCTAAGGATCCGTGGACGTTCGGTGGAGGCACCAAGCTGGAAATCAAA
Clones: 40A12-D2
Heavy chain VH
SEQ ID NOs: 71-80
<------------FR1------------> CDR1 <-----FR2----> CDR2 <---
QVQLKQSGPGLVQPSQSLSITCTVSGFSLTSYGIHWVRQSPGKGLEWLGVMWRGGSTDYNAPFMSRLSI
--------------FR3----------> CDR3 <---FR4--->
TKDNSKSQVFFKMNSLQADDTAIYYCAKSLITTGYAMDFWGQGTSVTVSS
Nucleotide sequence
CAGGTCCAGCTGAAGCAGTCAGGACCTGGCCTAGTGCAGCCCTCACAGAGCCTGTCCATAACCTGCACAGT
CTCTGGTTTCTCATTAACTAGCTATGGTATACACTGGGTTCGCCAGTCTCCAGGAAAGGGTCTGGAGTGGC
TGGGAGTGATGTGGAGAGGTGGAAGCACAGACTACAATGCACCTTTTATGTCCAGACTGAGCATCACCAAG
GACAACTCCAAGAGCCAAGTTTTCTTTAAAATGAACAGTCTGCAAGCTGATGACACTGCCATATATTACTG
TGCCAAAAGTCTGATTACGACTGGGTATGCTATGGACTTCTGGGGTCAAGGAACCTCAGTCACCGTCTCCT
CA
Light chain VK
<----------FR1--------> CDR1 <-----FR2-----> CDR2 <------------
DIQMTQSSSSFSVSLGDRVTITCKASEDIYNRLAWYQQKPGNAPRLLISGATSLETGVPSRFSGSGSGK
--------FR3-------> CDR3 <---FR4--->
DYTLSITSLQTEDVATYYCQQYWSNPYTFGGGTKLEIK
Nucleotide sequence
GACATCCAGATGACACAATCTTCATCCTCCTTTTCTGTATCTCTAGGAGACAGAGTCACCATTACTTGCAA
GGCAAGTGAGGACATATATAATCGGTTAGCCTGGTATCAGCAGAAACCAGGAAATGCTCCTAGGCTCTTAA
TATCTGGTGCAACCAGTTTGGAAACTGGGGTTCCTTCAAGATTCAGTGGCAGTGGATCTGGAAAGGATTAC
ACTCTCAGCATTACCAGTCTTCAGACTGAAGATGTTGCTACTTATTACTGTCAACAGTATTGGAGTAATCC
GTACACGTTCGGAGGGGGGACCAAGCTGGAAATAAAA
Clones: 36G11-E10
Heavy chain VH
SEQ ID NOs:81-90
<------------FR1------------> CDR1 <----FR2-----> CDR2 <---
QVQLKQSGPSPVQPSQSLSITCTVSGFSLTSYGVHWVRQSPGKGLEWLGVIWRGGSTDYNAVFMSRLSI
--------------FR3----------> CDR3 <----FR4--->
TKDNSKSQVFFKMNSLQTDDTAIYYCAKSLVTTGYTMDYWGQGTSVTVSS
Nucleotide sequence
CAGGTCCAGCTGAAGCAGTCAGGACCTAGCCCAGTGCAGCCCTCACAGAGCCTGTCCATAACCTGCACAGT
CTCTGGTTTCTCATTAACGAGCTATGGGGTACACTGGGTTCGCCAGTCTCCAGGAAAGGGTCTGGAGTGGC
TGGGAGTGATATGGAGAGGTGGAAGTACAGACTACAATGCAGTTTTCATGTCCAGACTGAGCATCACCAAG
GACAACTCCAAGAGCCAAGTTTTCTTTAAAATGAACAGTCTGCAAACTGATGACACTGCCATATACTACTG
TGCCAAAAGTCTTGTTACTACGGGCTATACTATGGACTACTGGGGTCAAGGAACCTCAGTCACCGTCTCCT
CA
Light chain VK
<----------FR1--------> CDR1 <-----FR2-----> CDR2 <------------
DIQMTQSSSSFSVSLGDRVTITCKASEDIYNRLAWYQQRPGNAPRLLISGTTSLETGVPSRFSGSGSGK
--------FR3-------> CDR3 <---FR4--->
DYTLSITSLQTEDVATYYCQQYWSIPWTFGGGTKLEIK
Nucleotide sequence
GACATCCAGATGACACAGTCTTCATCCTCCTTTTCTGTATCTCTAGGAGACAGAGTCACCATTACTTGCAA
GGCAAGTGAGGACATATATAATCGGTTAGCCTGGTATCAGCAGAGACCAGGAAATGCTCCTAGGCTCTTAA
TATCTGGTACAACCAGTTTGGAAACTGGGGTTCCTTCAAGATTCAGTGGCAGTGGATCTGGAAAGGATTAC
ACTCTCAGCATTACCAGTCTTCAGACTGAAGATGTTGCTACTTATTACTGTCAACAGTATTGGAGTATTCC
GTGGACGTTCGGTGGAGGCACCAAGCTGGAAATAAAA
Clones: 26C12-G9
Heavy chain VH
SEQ ID NOs:91-100
<------------FR1------------> CDR1 <-----FR2----> CDR2 <---
QVQLKQSGPGLVQPSQSLSIICTVSGFSLSSYGIHWVRQSPGKGLEWLGVIWRGGNTDYSVTFMSRLSI
--------------FR3----------> CDR3 <----FR4--->
TKDNSKSQVFFKMNSLQADDTAIYYCAKSLITTGYAMDYWGQGTSVTVSS
Nucleotide sequence
CAGGTCCAGCTGAAGCAGTCAGGACCTGGCCTAGTGCAGCCCTCACAGAGCCTGTCCATAATCTGCACAGT
CTCTGGTTTCTCCTTAAGTAGTTATGGTATTCACTGGGTTCGCCAGTCTCCAGGAAAGGGTCTGGAGTGGC
TGGGAGTGATATGGAGAGGTGGAAACACAGACTACAGTGTAACTTTCATGTCCAGACTGAGCATCACCAAG
GACAACTCCAAGAGCCAAGTTTTCTTTAAAATGAACAGTCTGCAAGCTGATGACACTGCCATATATTATTG
TGCCAAAAGTTTGATTACGACAGGGTATGCTATGGACTACTGGGGTCAAGGAACCTCAGTCACCGTCTCCT
CA
Light chain VK
<----------FR1--------> CDR1 <------FR2-----> CDR2 <------------
DIQMTQSSSSFSVSLGDRVTITCKASEDIYNRLAWYQQKPGTTPRLLISGATSLETGVPSRFSGSGSGK
---------FR3------> CDR3 <---FR4--->
DYTLSITSLQTEDVGTYYCQQYWSNPRTFGGGTKLEIK
Nucleotide sequence
GACATCCAGATGACACAATCTTCATCCTCCTTTTCTGTTTCTCTAGGAGACAGAGTCACCATTACTTGCAA
GGCAAGTGAAGACATATATAATCGGTTAGCCTGGTATCAGCAGAAACCAGGAACTACTCCTCGGCTCTTAA
TATCTGGTGCAACCAGTTTGGAAACTGGGGTTCCTTCAAGATTCAGTGGCAGTGGATCTGGAAAAGATTAC
ACTCTCAGCATTACCAGTCTTCAGACTGAAGATGTTGGTACTTATTACTGTCAACAGTATTGGAGTAATCC
GCGGACGTTCGGTGGAGGCACCAAGCTGGAAATAAAA
Clones: 20E12-C6
Heavy chain VH
SEQ ID NOs: 101-110
<-------------FR1-----------> CDR1 <----FR2-----> CDR2 <---
QVQLKQSGPGLVQPSQTLSITCTVSGFSLTSYGVHWVRQSPGKGLEWLGVIWRGGGTEYNAAFMSRLSI
------------FR3------------> CDR3 <----FR4--->
TKDNSKSQVFLKMNSLQADDTAIYYCAKSMITTGYAMDFWGQGTSVTVSS
Nucleotide sequence
CAGGTCCAGCTGAAGCAGTCAGGACCTGGCCTAGTGCAGCCCTCACAGACCCTGTCCATAACCTGCACAGT
CTCTGGTTTCTCATTAACTAGCTATGGTGTACACTGGGTTCGCCAGTCTCCAGGAAAGGGTCTGGAGTGGC
TGGGAGTGATATGGAGAGGTGGAGGCACAGAGTACAATGCAGCTTTCATGTCCAGACTGAGCATCACCAAG
GACAACTCCAAGAGCCAAGTTTTCTTAAAGATGAACAGTCTGCAAGCTGATGACACTGCCATATACTACTG
TGCCAAGTCGATGATTACGACAGGTTATGCTATGGACTTCTGGGGTCAAGGAACCTCAGTCACCGTCTCCT
CA
Light chain VK
<---------FR1---------> CDR1 <-----FR2-----> CDR2 <------------
DIQMTQSSSSFSVSLGDRVTITCKASEDIYKRLAWYQQKPGNAPRLLISGATSLETGIPSRFSGSGSGK
---------FR3------> CDR3 <---FR4--->
DYTLSLTSLQTEDVATYYCQQYWSSPRTFGGGAKLEIK
Nucleotide sequence
GACATCCAGATGACACAATCTTCATCCTCCTTTTCTGTATCTCTAGGAGACAGAGTCACCATCACTTGCAA
GGCAAGTGAGGACATATATAAGCGGTTAGCCTGGTATCAGCAGAAACCAGGAAATGCTCCTAGGCTCTTAA
TATCTGGTGCAACCAGTTTGGAAACTGGGATTCCTTCAAGATTCAGTGGCAGTGGATCTGGAAAGGATTAC
ACTCTCAGCCTTACCAGTCTTCAGACTGAAGATGTTGCTACTTATTACTGTCAACAGTATTGGAGTTCTCC
TCGGACGTTCGGTGGAGGCGCCAAGCTGGAAATAAAA
Clones: 22D3-F9
Heavy chain VH
SEQ ID NOs: 111-120
<------------FR1------------> CDR1 <-----FR2----> CDR2 <---
QVQLKQSGPGLVQPSQSLSITCTVSGFSLTTYGIHWIRQSPGRGLEWLGVIWRGGSTDYNAAFMSRLSF
-------------FR3-----------> CDR3 <---FR4--->
TKDNSKSQVFFKMNSLQADDTAIYYCAKSMITTGFTMDYWGQGTSVTVSS
Nucleotide sequence
CAGGTCCAGCTGAAGCAGTCAGGACCTGGCCTAGTGCAGCCCTCACAGAGCCTGTCCATAACCTGCACAGT
CTCTGGTTTCTCATTGACTACCTATGGTATACACTGGATTCGCCAGTCTCCTGGAAGGGGTCTGGAGTGGC
TGGGAGTGATTTGGAGAGGTGGAAGCACAGACTACAATGCAGCTTTCATGTCCAGACTGAGCTTCACCAAG
GACAACTCCAAGAGCCAAGTTTTCTTTAAAATGAACAGTCTGCAAGCTGATGACACTGCCATATACTACTG
TGCCAAAAGTATGATTACGACTGGATTTACTATGGACTACTGGGGTCAAGGAACCTCAGTCACCGTCTCCT
CAg
Light chain VK
<----------FR1--------> CDR1 <-----FR2-----> CDR2 <------------
DIQMTQSSSSFSVSLGDRVTITCRASEDIYNRLAWYQQKPGNAPRLLISGATSLETGVPSRFSGSGSGK
---------FR3------> CDR3 <---FR4--->
DYTLSITSLQTEDVATYYCQQFWSTPYTFGGGTKLEIK
Nucleotide sequence
GACATCCAGATGACACAATCTTCATCCTCCTTTTCTGTATCTCTAGGAGACAGAGTCACCATTACTTGCAG
GGCAAGTGAGGACATATATAACCGGTTAGCCTGGTATCAGCAGAAACCAGGAAATGCTCCTAGGCTCTTAA
TATCTGGTGCAACCAGTTTGGAAACTGGGGTTCCTTCAAGATTCAGTGGCAGTGGATCTGGAAAGGATTAC
ACTCTCAGCATTACCAGTCTTCAGACTGAAGATGTTGCTACTTATTATTGTCAACAGTTTTGGAGTACTCC
GTACACGTTCGGAGGGGGGACCAAGCTGGAAATAAAA
Clones: 12C7-A11
Heavy chain VH
SEQ ID NOs:121-130
<-------------FR1-----------> CDR1 <-----FR2----> CDR2 <---
QVQLKQSGPGLVQPSQSLSISCSVSGFSLTSYGVHWVRQSPGKGLEWLGVIWRGGSTDYNAAFSSRLSI
-------------FR3-----------> CDR3 <---FR4--->
TKDNSKSQVFFKMNSLQADDTAIYYCAKSMITTGRYFDVWGAGTTVTVSS
Nucleotide sequence
CAGGTCCAGCTGAAGCAGTCAGGACCTGGCCTAGTGCAGCCCTCACAGAGCCTGTCCATATCCTGCTCAGT
CTCTGGTTTCTCATTAACTAGTTATGGTGTACACTGGGTTCGCCAGTCTCCAGGAAAGGGTCTGGAGTGGC
TGGGAGTGATATGGAGAGGAGGAAGCACAGACTACAATGCAGCTTTCTCGTCCAGACTGAGCATCACCAAG
GACAACTCCAAGAGCCAAGTTTTCTTTAAAATGAACAGTCTGCAAGCTGATGACACTGCCATATATTACTG
TGCCAAATCTATGATTACGACGGGGAGGTACTTCGATGTCTGGGGCGCAGGGACCACGGTCACCGTCTCCT
CA
Light chain VK
<-----------FR1-------> CDR1 <------FR2-----> CDR2 <------------
DIQMTQSSSSFSVSLGDRVTITCKASEDIYNRLAWYQQKPGNAPRLLISGAASLETGIPSRFSGSGSGK
--------FR3-------> CDR3 <---FR4--->
NYTLSITSLQTEDVATYYCQQYWSTPWTFGGGTKLEIK
Nucleotide sequence
GACATCCAGATGACACAATCTTCATCCTCCTTTTCTGTATCTCTCGGAGACAGAGTCACCATTACTTGCAA
GGCAAGTGAGGACATATATAATCGGTTAGCCTGGTATCAGCAGAAACCAGGAAATGCTCCTAGGCTCTTAA
TATCTGGTGCAGCCAGTTTGGAAACTGGGATTCCTTCAAGATTCAGTGGCAGTGGATCTGGAAAGAATTAC
ACTCTCAGCATTACCAGTCTTCAGACTGAAGATGTTGCTACTTATTACTGTCAACAGTATTGGAGTACTCC
GTGGACGTTCGGTGGAGGCACCAAGCTGGAAATAAAA
Clones: 11F12-E1
Heavy chain VH
SEQ ID NOs: 131-140
<------------FR1------------> CDR1 <-----FR2----> CDR2 <---
QVQLKQSGPGLVQPSQSLSITCSVSGFSLTSYGVHWVRQSPGKGLEWLGVIWRGGSTDYNAAFMSRLSI
-------------FR3-----------> CDR3 <---FR4--->
TKDNSKSQVFFKMNSLQADDTAIYYCAKSMITTGRYFDVWGAGTTVTVSS
Nucleotide sequence
CAGGTCCAGCTGAAGCAGTCAGGACCTGGCCTAGTGCAGCCCTCACAGAGCCTGTCCATAACCTGCTCAGT
CTCTGGTTTCTCATTAACTAGCTATGGTGTACACTGGGTTCGCCAGTCTCCAGGAAAGGGTCTGGAGTGGC
TGGGAGTGATATGGAGAGGAGGAAGCACAGACTACAATGCAGCTTTCATGTCCAGACTGAGCATCACCAAG
GACAACTCCAAGAGCCAAGTTTTCTTTAAAATGAACAGTCTGCAAGCTGATGACACTGCCATATACTACTG
TGCCAAATCTATGATTACGACGGGGAGGTACTTCGATGTCTGGGGCGCAGGGACCACGGTCACCGTCTCCT
CA
Light chain VK
<----------FR1--------> CDR1 <------FR2-----> CDR2 <-------------
DIQMTQSSSSFSVSLGDRVTITCKASEDIYNRLAWYQQRPGNAPRLLISGATSLETGVPSRFSGSGSGK
-------FR3--------> CDR3 <---FR4--->
DYTLSITSLQTEDVATYYCQQYWSAPWTFGGGTKLEIK
Nucleotide sequence
GACATCCAGATGACACAATCTTCATCCTCCTTTTCTGTATCTCTCGGAGACAGAGTCACCATTACTTGCAA
GGCAAGTGAGGACATATATAATCGGTTAGCCTGGTATCAGCAGAGACCAGGAAATGCTCCTAGGCTCTTAA
TATCTGGTGCAACCAGTTTGGAAACTGGGGTTCCTTCAAGATTCAGTGGCAGTGGCTCTGGAAAGGATTAC
ACTCTCAGCATTACCAGTCTTCAGACTGAAGATGTTGCTACTTATTACTGTCAACAGTATTGGAGTGCTCC
GTGGACGTTCGGTGGAGGCACCAAGCTGGAAATAAAA
Clones: 23C4-D1
Heavy chain VH
SEQ ID NOs: 141-150
<-------------FR1-----------> CDR1 <-----FR2----> CDR2 <---
DVHLVESGGGLVQPGGSRKLSCAASGFTFSSFGMHWVRQAPERGLEWVAYISGDSNTIYYTDTVKGRFT
-------------FR3------------> CDR3 <---FR4--->
ISRDNPKNTLFLQMTSLRSEDTAMYYCAKSFDYAMDYWGQGTSVTVSS
Nucleotide sequence
GAtGTGCAtCTGGTGGAGTCTGGGGGAGGCTTAGTGCAGCCTGGAGGGTCCCGGAAACTCTCCTGTGCAGC
CTCTGGATTCACTTTCAGTAGCTTTGGAATGCACTGGGTTCGTCAGGCTCCAGAGAGGGGGCTGGAGTGGG
TCGCATATATCAGTGGTGACAGTAATACCATCTACTATACAGACACAGTGAAGGGCCGGTTCACCATCTCC
AGAGACAATCCCAAGAACACCCTGTTCCTGCAAATGACCAGTCTAAGGTCTGAGGACACGGCCATGTATTA
CTGTGCAAAATCGTTCGACTATGCTATGGACTACTGGGGTCAGGGAACCTCAGTCACCGTCTCCTCA
Light chain VK
<----------FR1--------> CDR1 <------FR2----> CDR2 <------
DIVMSQSPSSLAVSVGEKVTLSCKSSQSLLYSTNQKNYLAWYLQKPGQSPKLLIYWASTRESGVPDRFS
---------FR3---------> CDR3 <---FR4--->
GSGSGTDFALTINTVKAEDLAVYYCQQYYTYPYTFGGGTKLEIK
Nucleotide sequence
GACATTGTGATGTCACAGTCTCCATCCTCCCTAGCTGTGTCAGTTGGAGAGAAGGTTACTTTGAGCTGCAA
GTCCAGTCAGAGCCTTTTATATAGTACCAATCAAAAGAACTACTTGGCCTGGTACCTGCAGAAACCAGGGC
AGTCTCCTAAACTGCTGATTTACTGGGCATCCACTAGGGAATCTGGGGTCCCTGATCGCTTCTCAGGCAGT
GGATCTGGGACAGATTTCGCTCTCACCATCAATACTGTGAAGGCTGAAGACCTGGCAGTTTATTACTGTCA
GCAATATTATACCTATCCGTACACGTTCGGAGGGGGGACCAAGCTGGAAATAAAA
Clones: 29F3-G8
Heavy chain VH
SEQ ID NOs: 151-160
<------------FR1------------> CDR1 <----FR2-----> CDR2 <---
QVQLKQSGPGLVQPSQSLSITCTVSGFSLTSYGIHWLRQSPGKGLEWLGVIWRGGSTDYNAAFMSRLSI
------------FR3------------> CDR3 <---FR4--->
TKDNSKSQVFFKMNSLQADDTAIYYCAKMRVTTGFTMDYWGQGTSVTVSS
Nucleotide sequence
CAGGTCCAGCTGAAGCAGTCAGGACCTGGCCTAGTGCAGCCCTCACAGAGCCTGTCCATAACCTGCACAGT
CTCTGGTTTCTCATTAACTAGCTACGGTATACACTGGCTTCGCCAGTCTCCAGGAAAGGGTCTGGAGTGGC
TGGGAGTGATATGGAGAGGTGGAAGCACAGACTACAATGCAGCTTTCATGTCCAGACTGAGCATCACCAAG
GACAACTCCAAGAGCCAAGTTTTCTTTAAAATGAACAGTCTGCAAGCTGATGACACTGCCATATACTACTG
TGCCAAAATGAGAGTTACGACGGGGTTTACTATGGACTACTGGGGTCAAGGAACCTCAGTCACCGTCTCCT
CA
Light chain VK
<----------FR1--------> CDR1 <-----FR2-----> CDR2 <-------------
DIQMTQSSSSFSVSLGDRVTITCKASEDIYNRLAWYQQKPGNAPRLLISGATSLETGVPSRFSGSGSGK
--------FR3-------> CDR3 <---FR4--->
DYTLSITSLQTEDVATYYCQQYWSNPYTFGGGTKLEIK
Nucleotide sequence
GACATCCAGATGACACAATCTTCATCCTCCTTTTCTGTATCTCTAGGAGACAGAGTCACCATTACTTGCAA
GGCAAGTGAGGACATATATAATCGGTTAGCCTGGTATCAGCAGAAACCAGGAAATGCTCCTAGGCTCTTAA
TATCTGGTGCAACCAGTTTGGAAACTGGGGTTCCTTCAAGATTCAGTGGCAGTGGATCTGGAAAGGATTAC
ACTCTCAGCATTACCAGTCTTCAGACTGAAGATGTTGCTACTTATTACTGTCAACAGTATTGGAGTAATCC
GTACACGTTCGGAGGGGGGACCAAGCTGGAAATAAAA
Clones: 27F8
Heavy chain VH
SEQ ID NOs: 161-170
<-------------FR1-----------> CDR1 <----FR2-----> CDR2 <---
QVQLQQSGPELEKPGASVKISCKASGYSFTVYNINWVKQSNGKSLEWIGNIDPYFGGTIYNQKFKDKAT
--------------FR3-----------> CDR3 <---FR4--->
LTVDKSSSTAYMELKSLTSEDSAVYYCERSRGTWRFAYWGQGTLVTVSA
Nucleotide sequence
CAGGTCCAGCTGCAGCAGTCTGGACCTGAGTTGGAGAAGCCTGGCGCTTCAGTGAAGATATCCTGCAAGGC
TTCTGGTTACTCATTCACTGTCTACAACATAAACTGGGTGAAACAGAGCAATGGAAAGAGCCTTGAGTGGA
TTGGAAATATTGATCCTTACTTTGGTGGTACTATTTACAACCAGAAATTCAAGGACAAGGCCACATTGACT
GTCGACAAATCCTCCAGCACAGCCTACATGGAGCTCAAGAGCCTGACATCTGAGGACTCTGCAGTCTATTA
CTGTGAAAGATCGAGGGGGACGTGGAGGTTTGCTTACTGGGGCCAAGGGACTCTGGTCACTGTCTCTGCA
Light chain VK
<---------FR1---------> CDR1 <------FR2-----> CDR2 <------------
DIVMTQSPSSMSVSLGDTVTITCHASQGISSNIGWLQQKPGKSFKGLIYHGATLEDGIPSRFSGSGSGA
---------FR3---------> CDR3 <---FR4--->
DYSLTISSLDSEDFADYYCVQYAQFPYTFGGGTKLEIK
Nucleotide sequence
GACATTGTGATGACCCAGTCTCCATCCTCCATGTCTGTATCTCTGGGAGACACAGTCACCATCACTTGCCA
TGCAAGTCAGGGCATTAGCAGTAATATAGGGTGGTTGCAGCAGAAACCAGGGAAATCATTTAAGGGCCTGA
TCTATCATGGAGCCACCTTGGAAGATGGAATTCCATCAAGGTTCAGTGGCAGTGGATCTGGAGCAGATTAT
TCTCTCACCATCAGCAGCCTGGACTCTGAAGATTTTGCAGACTATTACTGCGTACAGTATGCTCAGTTTCC
GTACACGTTCGGAGGGGGGACCAAGCTGGAAATCAAA
Clones: 48H1-B11
Heavy chain VH
SEQ ID NOs: 171-180
<-------------FR1-----------> CDR1 <-----FR2----> CDR2 <---
EVQLKQSGPGLMQPSQSLSITCTVSGFSLTSYGIHWLRQSPGKGLEWLGVIWRGGSTDYNAAFMSRLSI
------------FR3------------> CDR3 <---FR4--->
TKDNSKSQVFFKMNSLQGDDTAIYYCAKGKVTTGFYFDFWGQGTTLTVSS
Nucleotide sequence
GAGGTGCAGCTGAAGCAGTCAGGACCTGGCCTAATGCAGCCCTCACAGAGCCTGTCCATAACCTGCACAGT
CTCTGGTTTCTCATTAACTAGCTATGGTATACACTGGCTTCGTCAGTCTCCAGGAAAGGGTCTGGAGTGGC
TGGGAGTGATATGGAGAGGTGGAAGCACAGACTACAATGCAGCTTTCATGTCCAGACTGAGCATCACCAAG
GACAACTCCAAGAGCCAAGTTTTCTTTAAAATGAACAGTCTGCAAGGTGATGACACTGCCATATACTACTG
TGCCAAAGGGAAGGTTACGACGGGGTTCTACTTTGACTTCTGGGGCCAAGGCACCACTCTCACAGTCTCCT
CA
Light chain VK
<---------FR1--------> CDR1 <-----FR2-----> CDR2 <------------
DIQMTQSSSSFSVSLGDRVTITCKASEDIYNRLVWYQQKPGNAPGLLISGVTSLETGVPSRFSGSGSGK
--------FR3-------> CDR3 <---FR4--->
DYTLTITSLQTEDVATYYCQQYWSTPYTFGGGTKLEIK
Nucleotide sequence
GACATCCAGATGACACAATCTTCATCCTCCTTTTCTGTATCTCTAGGAGACAGAGTCACCATTACTTGCAA
GGCAAGTGAGGACATATATAATCGGTTAGTCTGGTATCAGCAGAAACCAGGAAATGCTCCTGGGCTCTTAA
TATCTGGTGTAACCAGTTTGGAAACTGGAGTTCCTTCAAGATTCAGTGGCAGTGGATCTGGAAAGGATTAC
ACTCTCACTATTACCAGTCTTCAGACTGAAGATGTTGCTACTTATTACTGTCAACAGTATTGGAGTACTCC
GTACACGTTCGGAGGGGGAACCAAGCTGGAAATAAAA

The heavy chain variable region was cloned into a vector containing a human heavy chain constant region and regulatory elements to express the intact IgG heavy chain in mammalian cells. Similarly, the light chain variable region was cloned into a vector containing a human light chain constant region and regulatory elements to express the intact IgG light chain in mammalian cells. The vectors were verified by sequencing and then transfected into CHO-S mammalian cells, and IgG was expressed and secreted into the culture medium, the supernatant was combined and collected, purified after being filtered. IgG was purified by Protein A chromatography, the supernatant was loaded onto a Protein A column of appropriate size, washed with 50 mM Tris-HCl pH 8.0, 250 mM NaCl, and the bound IgG was eluted with 0.1 M Glycine-HCl (pH 3.0). The protein was ultrafiltered and concentrated using a concentmtion tube, OD280 was measured, and the concentration of IgG was determined by spectrophotometry.

4) CDC Activity of Chimeric Antibodies on Daudi Cells

Daudi cells were resuspended and plated into a 96-well plate at 50 μL/well and 50 μL of antibodies was added to each well. The humanized antibodies were diluted to 10 μg/mL as a starting concentration, and 3-fold serial dilutions was performed, with a total of 3 dilution series. 50 μL of complement mix was added to each well and the 96-well plate was placed in a 5% CO₂ incubator at 37° C. for 2 hours. Then 50 μL of 40% CCK-8 solution was added to each well, the plate was shaken for 10 seconds and incubated in a 5% CO₂ incubator for 3 hours. The 96-well plate was placed in a microplate reader (BioTek, Synergy HT) and OD450 values were read and the collected data were calculated using GraphPad Prism 5 software. The results were shown in FIGS. 3-1 and 3-2.

5) Inhibition of Chimeric Antibodies on CD38 Enzyme Activity

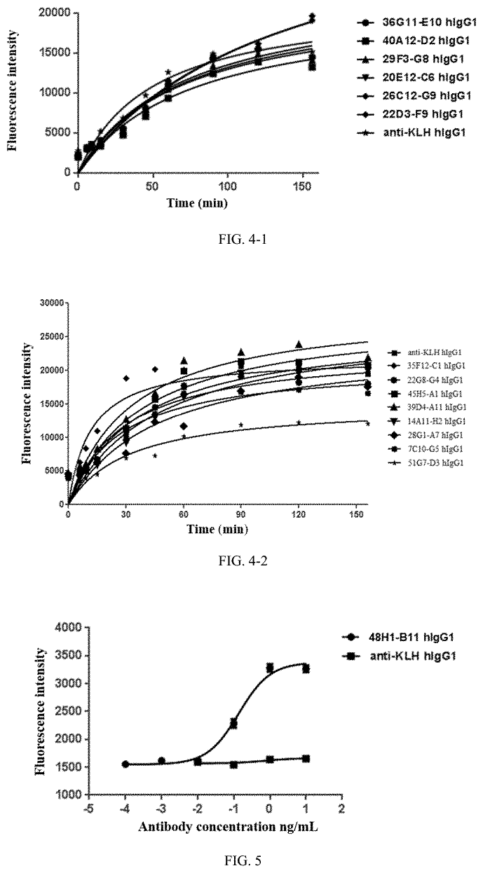

The synthesis and degradation of cyclic ADP-ribose (cADPR) were catalyzed by CD38, nicotinamide adenine dinucleotide (NAD+) was cyclized to cyclic adenosine-diphosphate-ribose (cADPR), which was hydrolyzed to ADPR. Due to the instability of cADPR, the catalytically produced cGDPR was often detected using nicotinamide guanine dinucleotide (NGD+) as a substrate in CD38 enzymatic activity studies. The anti-CD38 antibody may bind to CD38 and inhibit the production of cGDPR from NGD+ catalyzed by CD38 protease. The Daudi cells were mixed well with the serially diluted chimeric antibodies and incubated at room temperature for 15 minutes; NGD substrate was added at different time and mixed well for detection; the results were shown in Table 1 and FIGS. 4-1 and 4-2.

TABLE 1
Screening of CD38 chimeric antibodies for biological activity

| | | Inhibition of CD38 enzyme |
|---|---|---|
| CD38 antibody | CDC activity | activity |
| 21H12 G2 | − | + |
| 7C10-G5 | − | + |
| 15A1-G1 | − | + |
| 51G7-D3 | − | + |
| 17A4-H9 | + | + |
| 25C5-E9 | + | + |
| 25F8-C4 | + | + |
| 40A12-D2 | + | + |
| 36G11-E10 | + | + |
| 26C12-G9 | + | + |
| 20E12-C6 | + | + |
| 22D3-F9 | + | + |
| 12C7-A11 | + | + |
| 11F12-E1 | + | + |
| 23C4-D1 | + | + |
| 29F3-G8 | + | + |
| 27F8 | + | + |
| 48H1-B11 | ++ | + |

6) ADCC Activity of Chimeric Antibodies on Daudi Cells

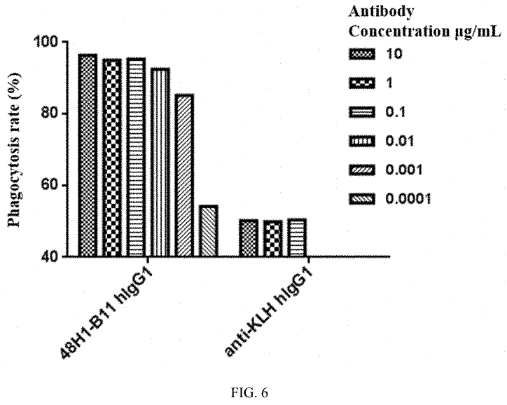

Daudi cells were plated at a density of 1×10⁶/mL in a U-shaped 96-well plate at 50 μL per well, 50 μL antibodies were added per well, and the plate was incubated in a 5% CO₂ incubator at 37° C. for 30 minutes. PBMC cells were fetched from a liquid nitrogen container, quickly thawed in water bath at 37° C., and quickly transferred into a 15 mL centrifuge tube, RPMI 1640 culture medium was added, followed by counting and centrifuging at 300*g for 10 minutes. The supernatant was discarded, and RPMI-1640 culture medium was resuspended to the cell density to 1×10⁷/mL; the 96-well plate was taken out, and 50 PBMC cells were added to each well, and the plate was incubated in a 5% CO₂ incubator for 4 hours. Thirty minutes prior to assay, 2 μL of cell lysate (100×) was added to the maximal well of target cells, and the mixture was cultured at 37° C. The culture solution was centrifuged at 300*g for 3 minutes, 50 μL of supernatant was added to a black ELISA plate, 50 μL of LDH detection substrate was added to each well (dissolving at room temperature in advance), and the plate was gently shaken and mixed well; after 10 minutes, the reaction was terminated by adding 25 μL stop solution to each well, and the plate was shaken for 10 seconds, followed by plate reading by selecting fluorescence (excitation wavelength of 560 nm, emission wavelength 590 nm). The results were shown in FIG. 5, wherein the chimeric antibodies 48H1-B11 had significant ADCC killing effect on Daudi cells in a dose-dependent manner.

7) ADCP Activity of Chimeric Antibodies on Daudi Cells

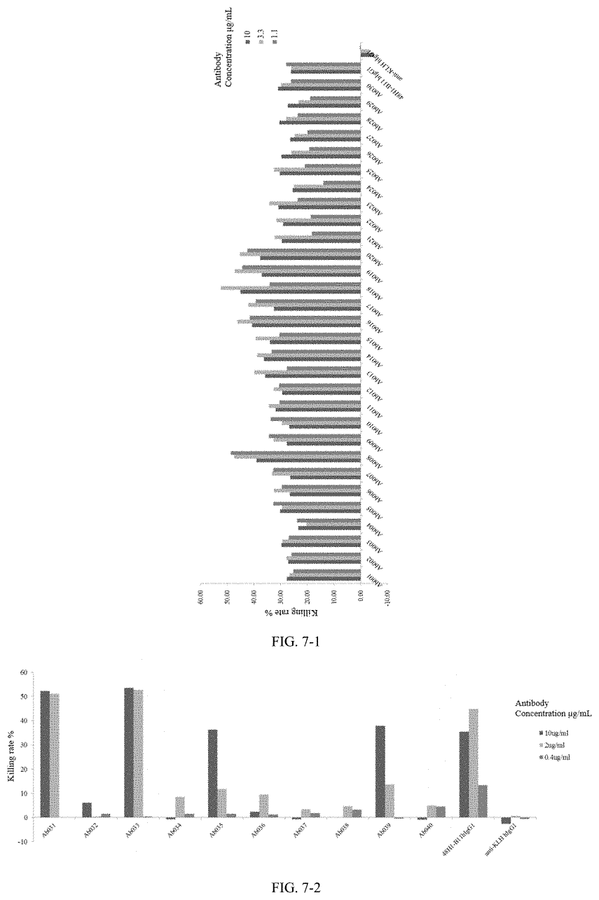

Daudi cells labeled with 1 μM CFSE were used as CD38+ target cells, 50 μL of the cells were seeded into a 96-well plate at 1×10⁵/well, the antibodies were diluted 4-fold, 50 μL of the antibodies were added to the plate, finally 100 μL macrophages induced to mature for 7 days with GM-CSF1 at a density of 2.5×10⁵/ml were added, with a ratio of macrophage:Daudi=1:4, the mixture was incubated in 5% CO₂ incubator at 37° C. for 4 hours, and finally the cells were collected and detected by flow cytometry. The results were shown in FIG. 6, wherein the chimeric antibodies 48H1-B11 had significant ADCP killing effect on Daudi cells in a dose-dependent manner.

### Example 5. Humanization of Anti-CD38 Antibody

mAb48H1-B11 murine antibody light chain was mouse IMGT_mVK13-84; firstly, the humanized framework region was screened, and IMGT_hVK1_39, IMGT_hVK_2 D_28, IMGT_hVK_3_7, IMGT_hVK_3_20, and IMGT_hVK_4_1 were selected respectively for CDR transplantation to obtain five humanized light chain variants h48H1-Vkv1-v5; the heavy chain of murine antibody was IGHV 2-5, and IMGT_hVH_1_69, IMGT_hVH_2_26, IMGT_hVH_3_23, IMGT_hVH_4_59, IMGT_hVH_5_51, and IMGT_hVH_7_4_1 were selected for CDR transplantation to obtain 6 humanized heavy chain variants h48H1-VHv1-v6. The light and heavy chain variants were paired in combination, then transiently expressed in CHO-S cells, and tested for affinity to the antibody and CDC activity.

Human IMGT_hVK3-20 with highest homology to light chain framework region of murine antibody mAb48H1-B11 from light and heavy chain variants completely retaining CDC activity of chimeric antibodies was selected for performing humanized sequence optimization, and human IGKJ4 with highest homology was selected for FM4; the human heavy chain germline gene IMGT_hVH 4-59 was selected for CDR transplantation, and human IGHJ4*01 with the highest homology was selected for FM4. In silico homology modeling was conducted, the CDR region and its surrounding framework amino acid was analyzed to avoid the concentrated distribution of molecular surface charge or hydrophobic region. A total of five heavy chain variants h48H1-VHv7-v11 and two light chain variants h48H1-VKv7-v8 were designed; after full-sequence synthesis of the light and heavy chains, respectively, they were cloned into eukaryotic expression vectors containing antibody kappa chain constant region Ckappa or human IgG1 constant region CH1-CH3; the light and heavy chain plasmids were paired in combination, then transfected into CHO-S cells and expressed at 37° C. for 5-6 days; the culture supernatant was collected and purified by Protein A column.

The humanized antibody heavy/light chain variable region sequences are as follows:

VH v1:
<-----------FR1---------> CDR1 <----FR2-----> CDR2 <---
QVQLVQSGAELKKPGSSVKVSCKVSGFSLTSYGIHWLRQAPGQGLEWMGVIWRGGSTDYNAKFQGRVTI
------------FR3------------> CDR3 <---FR4--->
TKDNSKSTVYMELSSLRSEDTAVYYCAKGKVTTGFYFDFWGQGTLVTVSS
Nucleotide sequence
CAGGTTCAGCTGGTTCAGTCTGGCGCCGAGCTGAAGAAACCTGGCAGCTCTGTGAAGGTGTCCTGCAAGGT
GTCCGGCTTCAGCCTGACAAGCTACGGCATCCACTGGCTGAGACAGGCCCCTGGACAAGGCTTGGAATGGA
TGGGAGTGATTTGGAGAGGCGGCAGCACCGACTACAACGCCAAGTTTCAGGGCAGAGTGACCATCACCAAG
GACAACAGCAAGAGCACCGTGTACATGGAACTGAGCAGCCTGAGAAGCGAGGACACCGCCGTGTACTACTG
CGCCAAGGGCAAAGTGACAACCGGCTTCTACTTCGACTTCTGGGGCCAGGGAACCCTGGTCACAGTCTCTT
CT
VH v2:
<------------FR1--------> CDR1 <-----FR2----> CDR2 <-----
QVTLKESGPVLVKPTETLTLTCTVSGFSLTSYGIHWLRQPPGKALEWLAVIWRGGSTDYNASLKSRLTISK
------------FR3----------> CDR3 <---FR4--->
DNSKSQVVLTMTNMDPVDTATYYCAKGKVTTGFYFDFWGQGTLVTVSS
Nucleotide sequence
CAAGTGACCCTGAAAGAAAGCGGCCCTGTGCTGGTCAAGCCCACCGAAACACTGACCCTGACCTGTACCGT
GTCCGGCTTCAGCCTGACAAGCTACGGAATCCACTGGCTGAGACAGCCTCCAGGCAAGGCTCTGGAATGGC
TGGCCGTTATTTGGAGAGGCGGCAGCACAGACTACAACGCCAGCCTGAAGTCCAGACTGACCATCAGCAAG
GACAACAGCAAGAGCCAGGTGGTGCTGACCATGACCAACATGGACCCTGTGGACACCGCCACCTACTACTG
CGCTAAGGGCAAAGTGACCACCGGCTTCTACTTCGACTTTTGGGGCCAGGGCACCCTGGTCACAGTCTCTT
CT
VH v3:
<-----------FR1---------> CDR1 <-----FR2----> CDR2 <-----
EVQLLESGGGLVQPGGSLRLSCAVSGFSLTSYGIHWLRQAPGKGLEWVSVIWRGGSTDYNASVKGRFTISK
------------FR3-----------> CDR3 <---FR4--->
DNSKSTVYLQMNSLRAEDTAVYYCAKGKVTTGFYFDFWGQGTLVTVSS
Nucleotide sequence
GAAGTGCAGCTGCTGGAATCTGGCGGAGGACTGGTTCAACCTGGCGGCTCTCTGAGACTGAGCTGTGCTGTGTCTGGCT
TCAGCCTGACCAGCTACGGAATCCACTGGCTGAGACAGGCCCCTGGCAAAGGACTGGAATGGGTGTCCGTGATTTGGAG
AGGCGGCAGCACAGACTACAACGCCTCTGTGAAGGGCAGATTCACCATCAGCAAGGACAACAGCAAGAGCACCGTGTAC
CTGCAGATGAACAGCCTGAGAGCCGAGGACACCGCCGTGTACTACTGTGCCAAGGGCAAAGTGACCACCGGCTTCTACT
TCGACTTTTGGGGCCAGGGCACCCTGGTCACAGTCTCTTCT
VH v4:
<------------FR1--------> CDR1 <-----FR2----> CDR2 <-----
QVQLQESGPGLVKPSETLSLTCTVSGFSLTSYGIHWLRQPPGKGLEWIGVIWRGGSTDYNASLKSRVTISK
-------------FR3----------> CDR3 <----FR4---->
DNSKSQVSLKLSSVTAADTAVYYCAKGKVTTGFYFDFWGQGTLVTVSS
Nucleotide sequence
CAGGTTCAGCTGCAAGAGTCTGGACCTGGCCTGGTCAAGCCTAGCGAGACACTGAGCCTGACCTGTACCGTGTCTGGCT
TCAGCCTGACCAGCTACGGAATCCACTGGCTGAGACAGCCTCCAGGCAAAGGCCTGGAATGGATCGGAGTGATTTGGAG
AGGCGGCAGCACCGACTACAACGCCAGCCTGAAGTCCAGAGTGACCATCAGCAAGGACAACAGCAAGAGCCAGGTGTCC
CTGAAGCTGAGCAGCGTGACAGCCGCTGATACCGCCGTGTACTACTGCGCCAAGGGCAAAGTGACCACCGGCTTCTACT
TCGACTTTTGGGGCCAGGGCACCCTGGTCACAGTCTCTTCT
VH v5:
<----------FR1----------> CDR1 <-----FR2-----> CDR2 <------
EVQLVQSGAELKKPGESLKISCKVSGFSLTSYGIHWLRQMPGKGLEWMGVIWRGGSTDYNASFQGQVTISK
-------------FR3---------> CDR3 <---FR4--->
DNSKSTVYLQWSSLKASDTAMYYCAKGKVTTGFYFDFWGQGTLVTVSS
Nucleotide sequence
GAAGTGCAGCTGGTTCAGTCTGGCGCCGAGCTGAAGAAGCCTGGCGAGAGCCTGAAGATCAGCTGCAAGGTGTCCGGCT
TCAGCCTGACCTCTTACGGCATCCACTGGCTGAGACAGATGCCTGGCAAAGGCCTGGAATGGATGGGAGTGATTTGGAG
AGGCGGCAGCACCGACTACAACGCCAGCTTTCAGGGCCAAGTGACCATCAGCAAGGACAACAGCAAGAGCACCGTGTAC
CTGCAGTGGTCCAGCCTGAAGGCCAGCGACACAGCCATGTACTACTGCGCCAAGGGCAAAGTGACAACCGGCTTCTACT
TCGACTTCTGGGGCCAGGGAACCCTGGTCACAGTCTCTTCT
VH v6:
<-----------FR1---------> CDR1 <-----FR2----> CDR2 <-----
QVQLVQSGSELKKPGASVKVSCKVSGFSLTSYGIHWLRQAPGQGLEWMGVIWRGGSTDYNAGFTGRFVISK
------------FR3----------> CDR3 <---FR4--->
DNSKSTVYLQICSLKAEDTAVYYCAKGKVTTGFYFDFWGQGTLVTVSS
Nucleotide sequence
CAGGTTCAGCTGGTGCAGTCTGGCAGCGAGCTGAAGAAACCTGGCGCCTCTGTGAAGGTGTCCTGCAAGGTGTCCGGCT
TCAGCCTGACAAGCTACGGCATCCACTGGCTGAGACAGGCCCCTGGACAAGGCTTGGAATGGATGGGAGTGATTTGGAG
AGGCGGCAGCACCGATTACAACGCCGGCTTCACAGGCAGATTCGTGATCAGCAAGGACAACAGCAAGAGCACCGTGTAC
CTGCAGATCTGTAGCCTGAAGGCCGAGGACACCGCCGTGTACTACTGTGCCAAGGGCAAAGTGACCACCGGCTTCTACT
TCGACTTTTGGGGCCAGGGCACCCTGGTCACAGTCTCTTCT
VH v7:
<------------FR1--------> CDR1 <-----FR2----> CDR2 <-----
QVQLQESGPGLVKPSETLSLTCTVSGFSLTSYGIHWIRQPPGKGLEWIGVIWRGGSTDYNPSLKSRVTISK
------------FR3----------> CDR3 <---FR4--->
DNSKSQVSLKLSSVTAADTAVYYCAKGKVTTGFYFDFWGQGTLVTVSS
Nucleotide sequence
CAGGTTCAGCTGCAAGAGTCTGGACCTGGCCTGGTCAAGCCTAGCGAGACACTGAGCCTGACCTGTACCGTGTCTGGCT
TCAGCCTGACCAGCTACGGAATCCACTGGatCAGACAGCCTCCAGGCAAAGGCCTGGAATGGATCGGAGTGATTTGGAG
AGGCGGCAGCACCGACTACAACCCCAGCCTGAAGTCCAGAGTGACCATCAGCAAGGACAACAGCAAGAGCCAGGTGTCC
CTGAAGCTGAGCAGCGTGACAGCCGCTGATACCGCCGTGTACTACTGCGCCAAGGGCAAAGTGACCACCGGCTTCTACT
TCGACTTTTGGGGCCAGGGCACCCTGGTCACAGTCTCTTCT
VH v8:
<-----------FR1---------> CDR1 <-----FR2----> CDR2 <-----
QVQLQESGPGLVKPSETLSLTCTVSGFSLTSYGIHWLRQPPGKGLEWIGVIWRGGSTDYNPSLKSRVTISK
------------FR3----------> CDR3 <----FR4---->
DTSKSQVSLKLSSVTAADTAVYYCAKGKVTTGFYFDFWGQGTLVTVSS
Nucleotide sequence
CAGGTTCAGCTGCAAGAGTCTGGACCTGGCCTGGTCAAGCCTAGCGAGACACTGAGCCTGACCTGTACCGTGTCTGGCT
TCAGCCTGACCAGCTACGGAATCCACTGGCTGAGACAGCCTCCAGGCAAAGGCCTGGAATGGATCGGAGTGATTTGGAG
AGGCGGCAGCACCGACTACAACCCCAGCCTGAAGTCCAGAGTGACCATCAGCAAGGACACCAGCAAGAGCCAGGTGTCC
CTGAAGCTGAGCAGCGTGACAGCCGCTGATACCGCCGTGTACTACTGCGCCAAGGGCAAAGTGACCACCGGCTTCTACT
TCGACTTTTGGGGCCAGGGCACCCTGGTCACAGTCTCTTCT
VH v9:
<------------FR1--------> CDR1 <-----FR2----> CDR2 <-----
QVQLQESGPGLVKPSETLSLTCTVSGFSLTSYGIHWIRQPPGKGLEWIGVIWRGGSTDYNPSLKSRVTISK
------------FR3----------> CDR3 <---FR4--->
DTSKSQVSLKLSSVTAADTAVYYCARGKVTTGFYFDFWGQGTLVTVSS
Nucleotide sequence
CAGGTTCAGCTGCAAGAGTCTGGACCTGGCCTGGTCAAGCCTAGCGAGACACTGAGCCTGACCTGTACCGTGTCTGGCT
TCAGCCTGACCAGCTACGGAATCCACTGGatCAGACAGCCTCCAGGCAAAGGCCTGGAATGGATCGGAGTGATTTGGAG
AGGCGGCAGCACCGACTACAACCCCAGCCTGAAGTCCAGAGTGACCATCAGCAAGGACACCAGCAAGAGCCAGGTGTCC
CTGAAGCTGAGCAGCGTGACAGCCGCTGATACCGCCGTGTACTACTGCGCCagaGGCAAAGTGACCACCGGCTTCTACT
TCGACTTTTGGGGCCAGGGCACCCTGGTCACAGTCTCTTCT
VH v10:
<-----------FR1---------> CDR1 <----FR2----> CDR2 <-----
QVQLQESGPGLVKPSETLSLTCTVSGFSLTSYGIHWIRQPPGKGLEWIGVIWAGGSTDYNPSLKSRVTISK
--------FR3----------------> CDR3 <---FR4--->
DTSKSQVSLKLSSVTAADTAVYYCARGKVTTGFYFDFWGQGTLVTVSS
Nucleotide sequence
CAGGTTCAGCTGCAAGAGTCTGGACCTGGCCTGGTCAAGCCTAGCGAGACACTGAGCCTGACCTGTACCGTGTCTGGCT
TCAGCCTGACCAGCTACGGAATCCACTGGatcAGACAGCCTCCAGGCAAAGGCCTGGAATGGATCGGAGTGATTTGGgc
tGGCGGCAGCACCGACTACAACcccAGCCTGAAGTCCAGAGTGACCATCAGCAAGGACaccAGCAAGAGCCAGGTGTCC
CTGAAGCTGAGCAGCGTGACAGCCGCTGATACCGCCGTGTACTACTGCGCCagaGGCAAAGTGACCACCGGCTTCTACT
TCGACTTTTGGGGCCAGGGCACCCTGGTCACAGTCTCTTCT
VH v11:
<----------FR1----------> CDR1 <-----FR2----> CDR2 <-----
QVQLQESGPGLVKPSETLSLTCTVSGFSLTSYGIHWIRQPPGKGLEWIGVIWRGGSTDYNPSLQSRVTISK
------------FR3----------> CDR3 <---FR4--->
DTSKSQVSLKLSSVTAADTAVYYCARGKVTTGFYFDFWGQGTLVTVSS
Nucleotide sequence
CAGGTTCAGCTGCAAGAGTCTGGACCTGGCCTGGTCAAGCCTAGCGAGACACTGAGCCTGACCTGTACCGTGTCTGGCT
TCAGCCTGACCAGCTACGGAATCCACTGGatCAGACAGCCTCCAGGCAAAGGCCTGGAATGGATCGGAGTGATTTGGAG
AGGCGGCAGCACCGACTACAACCCCAGCCTGCAGTCCAGAGTGACCATCAGCAAGGACACCAGCAAGAGCCAGGTGTCC
CTGAAGCTGAGCAGCGTGACAGCCGCTGATACCGCCGTGTACTACTGCGCCagaGGCAAAGTGACCACCGGCTTCTACT
TCGACTTTTGGGGCCAGGGCACCCTGGTCACAGTCTCTTCT
VK v1:
<---------FR1---------> CDR1 <-----FR2-----> CDR2 <--------------
DIQMTQSPSSLSASVGDRVTITCRASEDIYNRLVWYQQKPGKAPKLLISGVTSLETGVPSRFSGSGSGKDY
-------FR3------> CDR3 <---FR4--->
TLTISSLQPEDFATYYCQQYWSTPYTFGQGTKVEIK
Nucleotide sequence
GACATCCAGATGACACAGAGCCCTAGCAGCCTGTCTGCCAGCGTGGGAGACAGAGTGACCATCACCTGTAGAGCCAGCG
AGGACATCTACAACAGACTCGTGTGGTATCAGCAGAAGCCCGGCAAGGCTCCCAAGCTGCTGATTAGCGGAGTGACCAG
CCTGGAAACAGGCGTGCCAAGCAGATTCAGCGGCTCTGGCTCTGGCAAGGACTACACCCTGACCATCTCTAGCCTGCAG
CCTGAGGACTTCGCCACCTACTACTGCCAGCAGTACTGGTCTACCCCTTACACCTTCGGCCAGGGCACCAAGGTGGAAA
TCAAG
VK v2:
<--------FR1----------> CDR1 <-----FR2-----> CDR2 <--------------
DIVMTQSPLSLPVTPGEPASISCRASEDIYNRLVWYLQKPGQSPQLLISGVTSLETGVPDRFSGSGSGKDY
--------FR3-----> CDR3 <---FR4--->
TLKISRVEAEDVGVYYCQQYWSTPYTFGQGTKVEIK
Nucleotide sequence
GACATCGTGATGACACAGAGCCCTCTGAGCCTGCCTGTGACACCTGGCGAACCTGCCAGCATCAGCTGTAGAGCCAGCG
AGGACATCTACAACAGACTCGTGTGGTATCTGCAGAAGCCCGGCCAGTCTCCTCAGCTGCTGATTAGCGGAGTGACCAG
CCTGGAAACAGGCGTGCCAGACAGATTCAGCGGCTCTGGCTCTGGCAAGGACTACACCCTGAAGATCAGCAGAGTGGAA
GCCGAGGACGTGGGCGTGTACTACTGCCAGCAGTACTGGTCTACCCCTTACACCTTCGGCCAGGGCACCAAGGTGGAAA
TCAAG
VK v3:
<----------FR1--------> CDR1 <------FR2----> CDR2 <--------------
EIVMTQSPPTLSLSPGERVTLSCRASEDIYNRLVWYQQKPGQAPRLLISGVTSLETSIPARFSGSGSGKDY
-----FR3--------> CDR3 <---FR4--->
TLTISSLQPEDFAVYYCQQYWSTPYTFGQGTKVEIK
Nucleotide sequence
GAGATCGTGATGACACAGAGCCCTCCAACACTGTCTCTGAGCCCTGGCGAGAGAGTGACCCTGAGCTGTAGAGCCAGCG
AGGACATCTACAACAGACTCGTGTGGTATCAGCAGAAGCCCGGCCAGGCTCCTAGACTGCTGATTAGCGGAGTGACCAG
CCTGGAAACTAGCATCCCCGCCAGATTCAGCGGCTCTGGCTCTGGCAAGGACTACACCCTGACAATCAGCAGCCTGCAG
CCTGAGGACTTCGCCGTGTACTACTGCCAGCAGTACTGGTCTACCCCTTACACCTTCGGCCAGGGCACCAAGGTGGAAA
TCAAG
VK v4:
<----------FR1--------> CDR1 <-----FR2-----> CDR2 <--------------
EIVMTQSPGTLSLSPGERATLSCRASEDIYNRLVWYQQKPGQAPRLLISGVTSLETGIPDRFSGSGSGKDY
-------FR3------> CDR3 <---FR4--->
TLTISRLEPEDFAVYYCQQYWSTPYTFGQGTKVEIK
Nucleotide sequence
GAGATCGTGATGACACAGAGCCCCGGCACACTGTCACTGTCTCCAGGCGAAAGAGCCACACTGAGCTGTAGAGCCAGCG
AGGACATCTACAACAGACTCGTGTGGTATCAGCAGAAGCCCGGCCAGGCTCCTAGACTGCTGATTAGCGGAGTGACCAG
CCTGGAAACAGGCATCCCCGACAGATTCAGCGGCTCTGGCTCTGGCAAGGACTACACCCTGACCATCAGCAGACTGGAA
CCCGAGGACTTCGCCGTGTACTACTGCCAGCAGTACTGGTCTACCCCTTACACCTTCGGCCAGGGCACCAAGGTGGAAA
TCAAG
VK v5:
<---------FR1---------> CDR1 <-----FR2-----> CDR2 <--------------
DIVMTQSPDSLAVSLGERATINCKASEDIYNRLVWYQQKPGQPPKLLISGVTSLETGVPDRFSGSGSGKDY
--------FR3-----> CDR3 <---FR4--->
TLTISSLQAEDVAVYYCQQYWSTPYTFGQGTKVEIK
Nucleotide sequence
GACATCGTGATGACACAGAGCCCTGATAGCCTGGCCGTGTCTCTGGGAGAGAGAGCCACCATCAACTGCAAGGCCAGCG
AGGACATCTACAACAGACTCGTGTGGTATCAGCAGAAGCCCGGCCAGCCTCCTAAGCTGCTGATTAGCGGAGTGACCAG
CCTGGAAACAGGCGTGCCAGACAGATTCAGCGGCTCTGGCTCTGGCAAGGACTACACCCTGACAATCAGCTCCCTGCAG
GCCGAGGATGTGGCCGTGTACTACTGCCAGCAGTACTGGTCTACCCCTTACACCTTCGGCCAGGGCACCAAGGTGGAAA
TCAAG
VK v7:
<----------FR1--------> CDR1 <-----FR2-----> CDR2 <--------------
EIVMTQSPPTLSLSPGERVTLSCRASEDIYNRLVWYQQKPGQAPRLLISGVTSLETSIPARFSGSGSGTDY
-----FR3--------> CDR3 <--FR4--->
TLTISSLQPEDFAVYYCQQYWSTPYTFGQGTKVEIK
Nucleotide sequence
GAGATCGTGATGACACAGAGCCCTCCAACACTGTCTCTGAGCCCTGGCGAGAGAGTGACCCTGAGCTGTAGAGCCAGCG
AGGACATCTACAACAGACTCGTGTGGTATCAGCAGAAGCCCGGCCAGGCTCCTAGACTGCTGATTAGCGGAGTGACCAG
CCTGGAAACTAGCATCCCCGCCAGATTCAGCGGCTCTGGCTCTGGCacaGACTACACCCTGACAATCAGCAGCCTGCAG
CCTGAGGACTTCGCCGTGTACTACTGCCAGCAGTACTGGTCTACCCCTTACACCTTCGGCCAGGGCACCAAGGTGGAAA
TCAAG
VK v8:
<----------FR1--------> CDR1 <-----FR2-----> CDR2 <--------------
EIVMTQSPPTLSLSPGERVTLSCRASEDIYNYLVWYQQKPGQAPRLLISGVTSLETSIPARFSGSGSGTDY
-------FR3------> CDR3 <--FR4--->
TLTISSLQPEDFAVYYCQQYWSTPYTFGQGTKVEIK
Nucleotide sequence
GAGATCGTGATGACACAGAGCCCTCCAACACTGTCTCTGAGCCCTGGCGAGAGAGTGACCCTGAGCTGTAGAGCCAGCG
AGGACATCTACAACtacCTCGTGTGGTATCAGCAGAAGCCCGGCCAGGCTCCTAGACTGCTGATTAGCGGAGTGACCAG
CCTGGAAACTAGCATCCCCGCCAGATTCAGCGGCTCTGGCTCTGGCacaGACTACACCCTGACAATCAGCAGCCTGCAG
CCTGAGGACTTCGCCGTGTACTACTGCCAGCAGTACTGGTCTACCCCTTACACCTTCGGCCAGGGCACCAAGGTGGAAA
TCAAG

### Example 6. CDC Killing of Different Humanized CD38 Antibodies on Tumor Cells Daudi

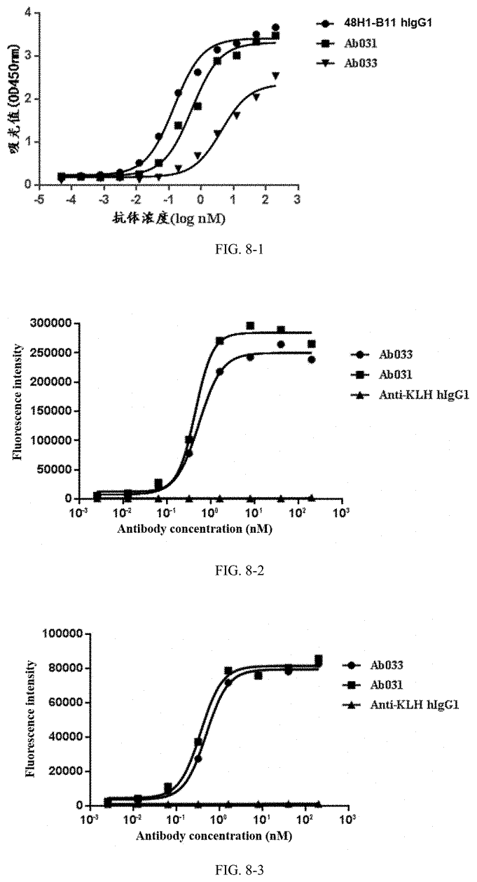

Different cells were seeded into a U-shaped 96-well plate, antibodies of different concentrations were added into each well, and the plate was placed in a CO₂ incubator for half an hour; 50 μl of complement mix was then added to each well and incubated in a CO₂ incubator for 2 hours. Then, 50 μl CCK-8 solution was added into each well, followed by shaking and blending, and culturing for another 3 hours; the light absorption OD450 was selected for plate reading. The results were shown in FIGS. 7-1 and 7-2. Among different humanized CD38 antibodies, humanized variants Ab031 and Ab033 have the highest maximum killing rate on Dauai cells.

TABLE 2
CDC killing of different humanized CD38 antibodies on tumor cells Daudi

| Humanized variants | VH | VK | CDC |
|---|---|---|---|
| Ab001 | h48H1-VHv1 | h48H1-VKv1 | + |
| Ab002 | h48H1-VHv1 | h48H1-VKv2 | + |
| Ab003 | h48H1-VHv1 | h48H1-VKv3 | + |
| Ab004 | h48H1-VHv1 | h48H1-VKv4 | + |
| Ab005 | h48H1-VHv1 | h48H1-VKv5 | + |
| Ab006 | h48H1-VHv2 | h48H1-VKv1 | + |
| Ab007 | h48H1-VHv2 | h48H1-VKv2 | + |
| Ab008 | h48H1-VHv2 | h48H1-VKv3 | ++ |
| Ab009 | h48H1-VHv2 | h48H1-VKv4 | + |
| Ab010 | h48H1-VHv2 | h48H1-VKv5 | + |
| Ab011 | h48H1-VHv3 | h48H1-VKv1 | + |
| Ab012 | h48H1-VHv3 | h48H1-VKv2 | + |
| Ab013 | h48H1-VHv3 | h48H1-VKv3 | + |
| Ab014 | h48H1-VHv3 | h48H1-VKv4 | + |
| Ab015 | h48H1-VHv3 | h48H1-VKv5 | + |
| Ab016 | h48H1-VHv4 | h48H1-VKv1 | ++ |
| Ab017 | h48H1-VHv4 | h48H1-VKv2 | + |
| Ab018 | h48H1-VHv4 | h48H1-VKv3 | ++ |
| Ab019 | h48H1-VHv4 | h48H1-VKv4 | ++ |
| Ab020 | h48H1-VHv4 | h48H1-VKv5 | ++ |
| Ab021 | h48H1-VHv5 | h48H1-VKv1 | + |
| Ab022 | h48H1-VHv5 | h48H1-VKv2 | + |
| Ab023 | h48H1-VHv5 | h48H1-VKv3 | + |
| Ab024 | h48H1-VHv5 | h48H1-VKv4 | + |
| Ab025 | h48H1-VHv5 | h48H1-VKv5 | + |
| Ab026 | h48H1-VHv6 | h48H1-VKv1 | + |
| Ab027 | h48H1-VHv6 | h48H1-VKv2 | + |
| Ab028 | h48H1-VHv6 | h48H1-VKv3 | + |
| Ab029 | h48H1-VHv6 | h48H1-VKv4 | + |
| Ab030 | h48H1-VHv6 | h48H1-VKv5 | + |
| Ab031 | h48H1-VHv7 | h48H1-VKv7 | ++ |
| Ab032 | h48H1-VHv7 | h48H1-VKv8 | − |
| Ab033 | h48H1-VHv8 | h48H1-VKv7 | ++ |
| Ab034 | h48H1-VHv8 | h48H1-VKv8 | − |
| Ab035 | h48H1-VHv9 | h48H1-VKv7 | + |
| Ab036 | h48H1-VHv9 | h48H1-VKv8 | − |
| Ab037 | h48H1-VHv10 | h48H1-VKv7 | − |
| Ab038 | h48H1-VHv10 | h48H1-VKv8 | − |
| Ab039 | h48H1-VHv11 | h48H1-VKv7 | + |
| Ab040 | h48H1-VHv11 | h48H1-VKv8 | − |

### Example 7 Determination of Affinity for Humanized CD38 Antibodies

1) Affinity Assay by ELISA

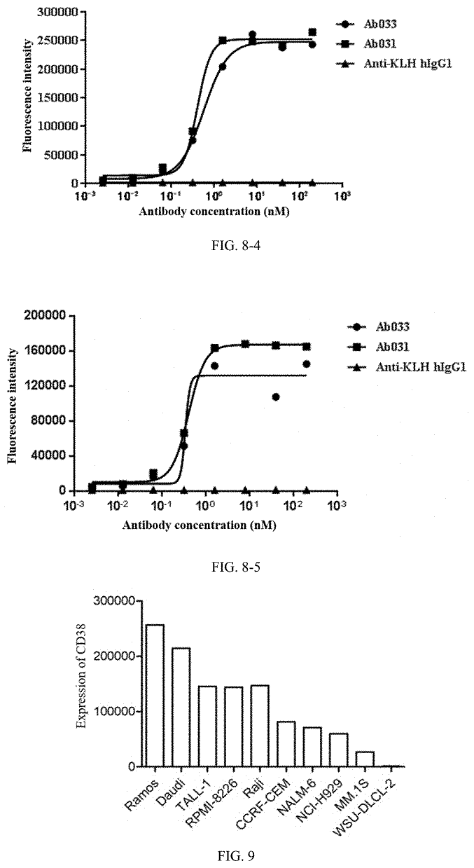

A 96-well plate was coated with human CD38 protein and incubated overnight at 4° C. The solution in wells were then discarded, washed 3 times with wash buffer, and blocked by the addition of 2% milk in PBS for 60 minutes. After washing 3 times with washing buffer, 100 μL of antibodies at different dilutions were added to each well, and the mixture was incubated at 37° C. for 1 hour; after washing 3 times with washing buffer, HPR-labeled goat anti-human IgG Fc was diluted at 1:5000 with 0.5% BSA, and the mixture was incubated at room temperature for 1 hour; after washing 3 times with washing buffer, 100 μL of TMB substrate solution was added for color development; after reaction at 37° C. for 10 minutes, the reaction was terminated with 50 μL of 2M sulfuric acid solution and the absorbance was read at 450 nm. The results were shown in FIG. 8-1 and Table 3, humanized CD38 antibodies Ab031 (EC50=0.51 nM) and Ab033 (EC50=4.24 nM) bound to CD38 with high affinity according to ELISA.

TABLE 3
Affinity Assay by ELISA

| Antibody | EC50 (nM) |
|---|---|
| Ab031 | 0.51 |
| Ab033 | 4.24 |

2) Affinity Assay by Biacore

The humanized variant of 0.5 μg/ml was captured on the surface of protein A chip with a 1:1 monovalent binding mode, the signal was captured at about 200 RU, human CD38 recombinant protein at different concentrations flowed through the chip surface as an analyte, and the change of SPR signal during the process of antibody binding and dissociation was recorded and fitted with Langmuir 1:1 kinetics theoretical model to analyze the goodness of fit with the model and provide affinity data. As shown in Table 4, the humanized antibody Ab031 has an affinity of 0.28 nM and Ab033 has an affinity of 1.43 nM according to Biacore assay.

TABLE 4
Affinity Assay by Biacore

| Antibody | Ka(1/Ms) | Kd(1/s) | KD(M) |
|---|---|---|---|
| Ab031 | 2.00E+06 | 5.56E−04 | 2.78E−10 |
| Ab033 | 3.71E+06 | 5.32E−03 | 1.43E−09 |

3) Cell Affinity Assay by FACS

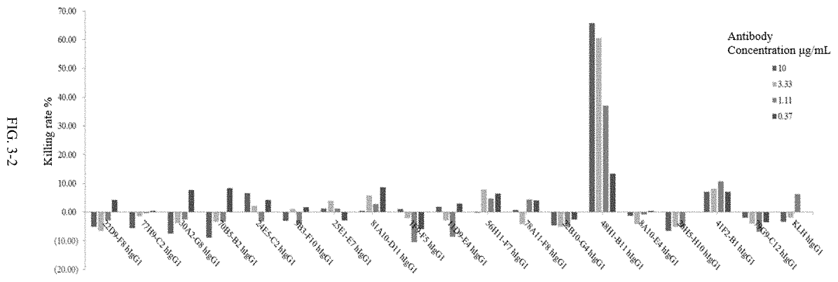

Different cells in logarithmic phase were blocked with 3% BSA for 30 minutes, and then plated into a U-shaped 96-well plate at 5×10⁴ cells/100 μL; centrifugation was conducted at 1100 rpm for 3 minutes, the supernatant was discarded, the cells were patted and loosed gently, 50 μL of serially diluted antibody (diluted 3-fold in 5 series, with an initial concentration of 30 μg/mL) was added to each well, and the mixture was incubated at 4° C. for 1 hour. After the incubation, 180 μL 0.5% BSA was added to each well, wash 3 times, 30 μL/well of secondary antibody Alexa Fluro 647 anti-human IgG (Jackson ImmunoResearch, Cat: 109-606-170) was added, and the mixture was incubated at 4° C. for 45 minutes. After the incubation, each well was washed 3 times with 180 μL 0.5% BSA, and finally resuspended in 50 μL PBS each well for iQue (Intellicyt, USA) assay. As shown in FIG. 8-2 and Table 5, according to FACS, the humanized antibodies Ab031 and Ab033 bind to CD38 molecules expressed on Daudi with a high affinity; meanwhile, the humanized variants Ab031 and Ab033 also recognize CD38 molecules expressed on other tumor cells NALM-6, Ramos.2G6.4C10, and TALL-1 (FIGS. 8-3, 8-4, and 8-5).

TABLE 5
Cell Affinity Assay by FACS

| | Antibody EC₅₀ (nM) | |
|---|---|---|
| Cell | Ab031 | Ab033 |
| Daudi | 0.44 | 0.54 |
| NALM-6 | 0.37 | 0.49 |
| Ramos.2G6.4C10 | 0.42 | 0.58 |
| TALL-1 | 0.41 | 0.34 |

### Example 8. Expression of CD38 on Different Tumor Cells

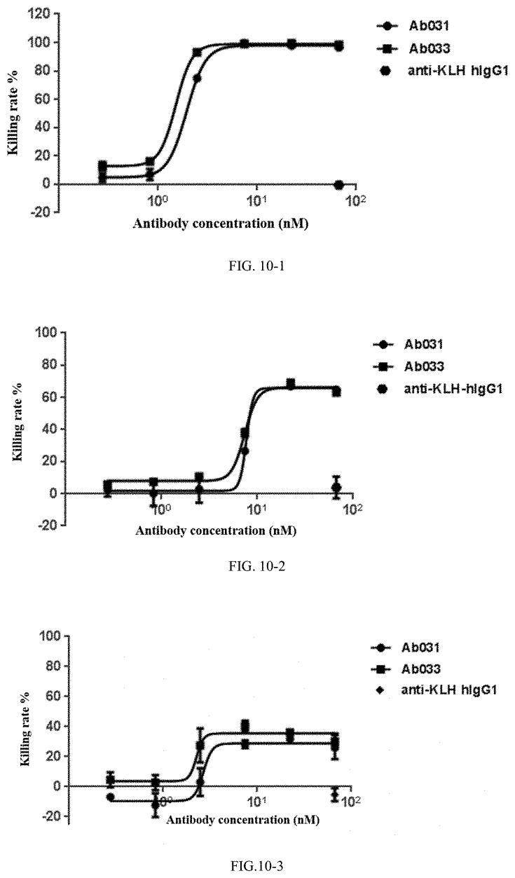

The expression of CD38 on different tumor cells was detected by FACS analysis. 1*10⁶ different tumor cells were centrifugated and collected, then washed once with 3 mL PBS; after blocking for 20 minutes, 50 μl of diluted primary antibody solution anti-human CD38 hIgGl-biotin was added and incubated on ice for 40 minutes; after washing twice, and 100 μl diluted secondary antibody solution SA-PE (Jackson, Cat. No: 016110084) was added, the mixture was incubated on ice for 30 minutes in the dark, washed twice, and centrifuged to collect cells; 200 μl of PI diluent was added to each well, the mixture was pipetted gently and mixed well, 500 μl of 0.5% BSA/PBS solution was added into a standard BD Qusntibrite PE tube (BD, Cat. No: 340495) in the dark, followed by incubation on ice and performing flow cytometry. As shown in FIG. 9, high expression of CD38 was detected on Daudi and Ramos.2G6.4C10 (ATCC CRL-1923) cells; medium expression of CD38 was detected on RPMI-8226 (ATCC CRM-CCL-155), TALL-1 (Shanghai Sixin Biotechnology Co., Ltd.), and Raji (National Institutes for Food and Drug Control) cells; low expression of CD38 was detected on NCI-H929 (Nanjing Cobioer Biosciences), CCRF-CEM, and NALM-6 (Shanghai Zishi Biotechnology Co., Ltd.) cells; and weak expression of CD38 was detected on MM.1S (ATCC CRL-2974), MOLP-8, and WSU-DLCL-2 (Shanghai Sixin Biotechnology Co., Ltd.) cells.

### Example 9. CDC Killing of Humanized CD38 Antibodies on Different Tumor Cells

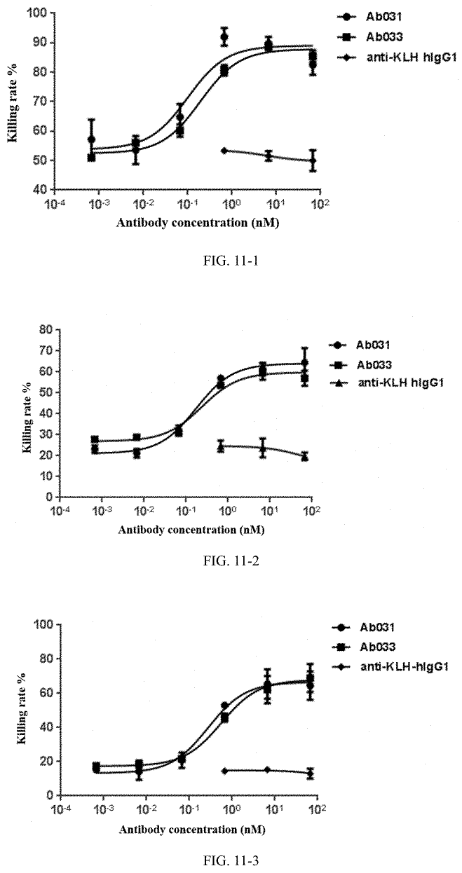

Different cells were seeded into a U-shaped 96-well plate, antibodies of different concentrations were added into each well, and the plate was placed in a CO₂ incubator for half an hour; 50 μl of complement mix was then added to each well and incubated in a CO₂ incubator for 2 hours. Then, 50 μl CCK-8 solution was added into each well, followed by shaking and blending, and culturing for another 3 hours; the light absorption OD450 was selected for plate reading. As shown in FIGS. 10-1, 10-2, and 10-3, the humanized antibodies Ab031 and Ab033 had a maximum killing rate of 98% for Ramos.2G6.4C10 cells, 65% for Daudi cells, and 40% for TALL-1 cells.

### Example 10. ADCC Killing of Humanized CD38 Antibodies on Different Tumor Cells

Different cells were seeded into a U-shaped 96-well plate, antibodies of different concentrations were added into each well, and the plate was placed in a CO₂ incubator for half an hour; 5×10⁵ PBMC cells were then added to each well and incubated in a CO₂ incubator for 4 hours. 30 minutes before detection, cell lysate was added and the mixture was cultured, followed by centrifuging to collect the supernatant into a black ELISA plate, a LDH detection substrate was added, followed by shaking and evenly blending After 10 minutes, the stop solution was added to terminate the reaction, followed by shaking and evenly blending; fluorescence was selected to read the plate, and killing rate was calculated. The calculation equation for killing rate was as follows:

$$Killing rate \% = \frac{Experimental wells - Controls wells}{Maximum release wells - Control wells} \times 100 \%$$

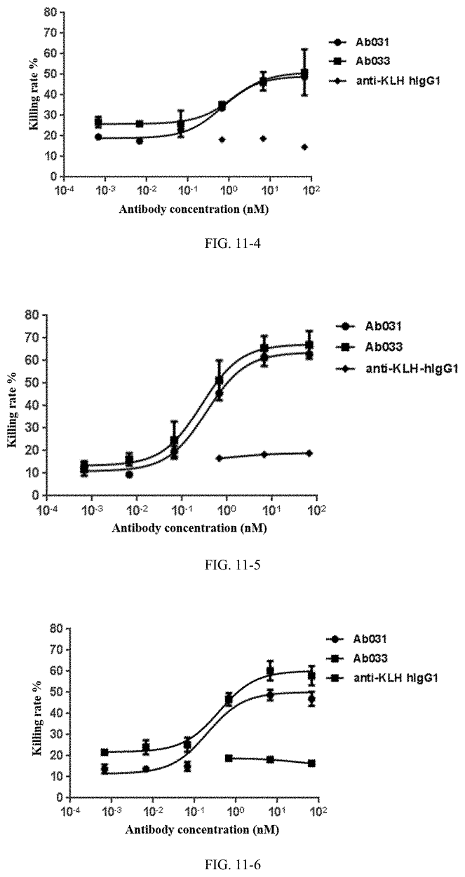

As shown in FIGS. 11-1, 11-2, 11-3, 11-4, 11-5, 11-6, 11-7, and 11-8, the humanized antibodies Ab031 and Ab033 had a killing rate of 90% for Ramos.2G6.4C10 cells, 65% for NALM-6 cells, 67% for RPMI-8226 cells, and 48% for CRFF-CEM cells; furthermore, 62% for Daudi cells, 40% for TALL-1 cells, 20% for MAUS cells, 48% for NCI-H929 cells, and 75% for Raji cells.

### Example 11. ADCP Killing of Humanized CD38 Antibodies on Different Tumor Cells

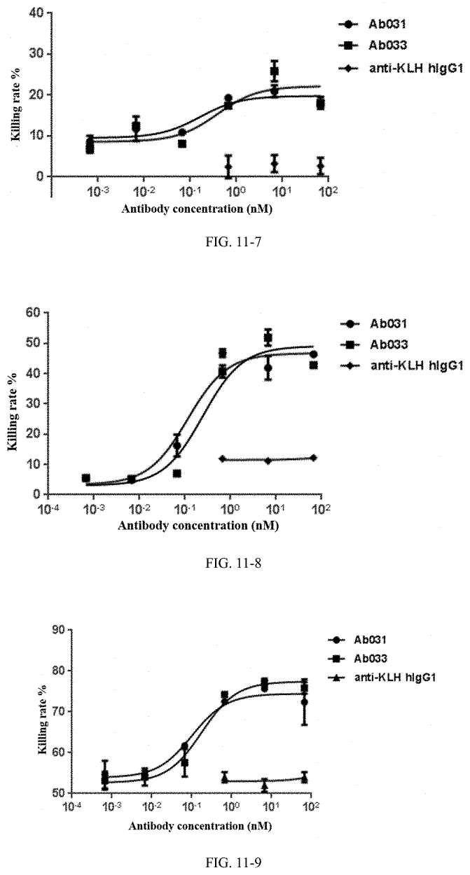

1 μM CFSE labeled Daudi cells as CD38+ target cells were seeded into a 96-well plate, the serially diluted antibody was added, the mixture was incubated in CO₂ incubator, and finally GM-CSF1 was added to induce mature macrophages in a proportion of macrophages:Daudi=1:4, and the mixture was incubated in a CO₂ incubator for 4 hour; the diluted solution of anti-human CD14 APC antibody was added, followed by standing on ice in the dark for 20 minutes; the collect cells were detected with a flow cytometer, single positive cell population of CD14 was gated in macrophage alone wells as control, single positive cell population of CD14 and double positive cell population of CD14 and CSFL were gated in experimental wells, and the number of cells in two groups was counted. As shown in FIGS. 12-1, 12-2, 12-3, 12-4s and 12-5, the humanized CD38 antibodies Ab031 and Ab033 had varying degrees of ADCP phagocytosis on CD38-expressing cells Daudi, TALL-1, Ramos.2G6.4C10, Raji, and NALM-6.

### Example 12. Inhibition of Humanized CD38 Antibodies on CD38 Enzymatic Activity

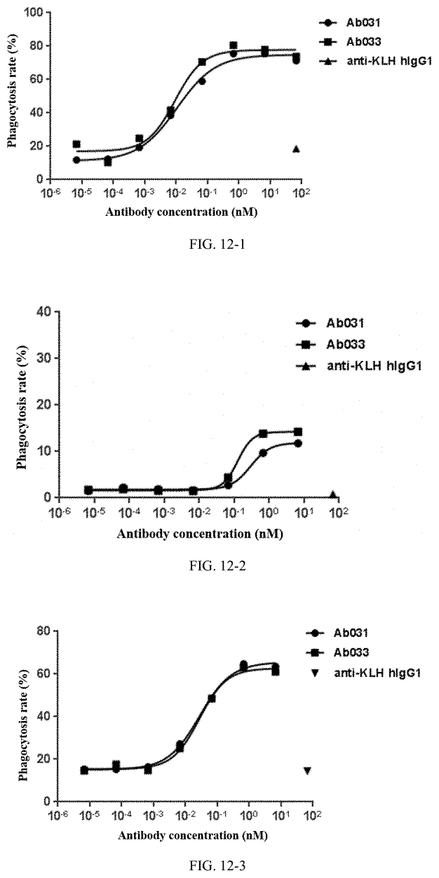

CD38 recombinant protein was mixed with the serially diluted humanized CD38 antibodies Ab031 and Ab0337, and the mixture was incubated at room temperature for 15 minutes; NGD substrate was added at different time points, and the mixture was evenly blended for detection; as shown in FIG. 13, both of the chimeric antibody 48H1-B11 and humanized CD38 antibodies Ab031 and Ab033 had significant inhibitory effect on CD38 enzyme activity.

### Example 13. Apoptosis Induced by Humanized CD38 Antibodies

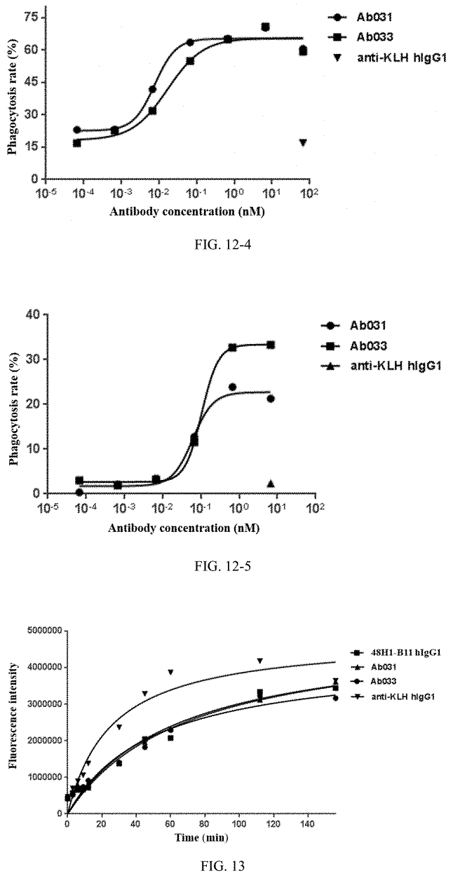

2*10⁵ Daudi cells were seeded in a 24-well plate and cultured at 37° C. overnight; 100 μl of the diluted and mixed humanized antibodies were added into each well, and the plate was placed in a CO₂ incubator to culture; 24 hours or 48 hours after cells were induced and stimulated, the cells were collected, 100 μl Annexin V-FITC incubation buffer was added, the mixture was blended gently, then propidium iodide staining solution was added, and the mixture was blended gently. The mixture was incubated in the dark for 15 minutes at room temperature, and flow cytometry analysis indicated that early and late apoptotic cells stained with Annexin V-FITC showed green fluorescence (positive); late apoptotic or dead cells stained with PI showed red fluorescence. As shown in FIG. 14, both of the humanized CD38 antibodies Ab031 and Ab033 could detect the apoptosis of Daudi cells.

### Example 14. Lysis of Human Erythrocytes Induced by Humanized CD38 Antibodies

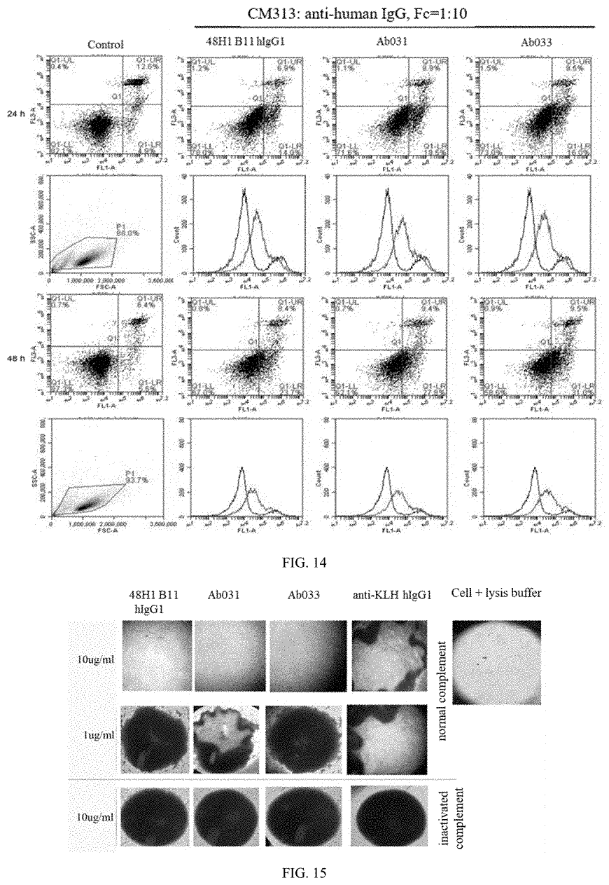

Human red blood cells were plated in a 96-well plate, 50 μl of serially diluted antibodies with different concentrations (10 μg/mL and 1 μg/mL) were added, then 50 μl of serially diluted complement was added, the mixture was placed in a CO₂ incubator for cultivation for 2 hours with inactivated complement as control, then the state of cells under microscope was observed. As shown in FIG. 15, neither the humanized CD38 antibodies Ab031 nor Ab033 caused lysis of erythrocytes in control and experimental groups.

### Example 15. Anti-Tumor Effect of Humanized CD38 Antibodies in Daudi Cell Line Subcutaneous Xenograft NOD/SCID Mouse Model

Raji cells were cultured in a RPMI 1640 medium containing 10% fetal bovine serum. Raji cells in logarithmic phase were collected, resuspended in PBS to the appropriate concentration and mixed with matrigel at 1:1 for subcutaneous tumor inoculation in NOD/SCID mice. Female mice were inoculated subcutaneously on the right side with 1×10⁷ Raji cells, randomly grouped according to tumor size when the mean tumor volume was 100 mm³. Intravenous administration in mice were performed via the tail vein twice weekly at a dose of 10 mg/kg for two weeks in each group of 6 mice, and tumor volume was monitored in the treated groups. Tumor volume was calculated as: tumor volume (mm³)=1/2 ×(a×b²) (where a represents long diameter and b represents short diameter).

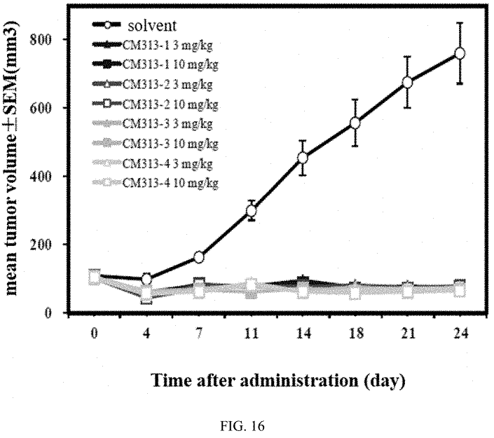

As shown in FIG. 16, all of the CD38 antibodies (3/10 mg/kg, IP, DO, 3) significantly inhibited the production of subcutaneous xenograft tumors in mice with human B-cell lymphoma Daudi, resulting in partial regression of tumor, wherein CM313-1 was humanized antibody Ab031, CM313-2 was humanized antibody Ab033, CM313-3 was antibody −005 (US 20150231235A1), and CM313-4 was 38SB19 (US 20110293606A1).

1. An antibody or antigen-binding portion thereof that binds to CD38, comprising heavy chain CDRs selected from the amino acid sequences of SEQ ID NOs: 2-4, 12-14, 22-24, 32-34, 42-44, 52-54, 62-64, 72-74, 82-84, 92-94, 102-144, 112-114, 122-124, 132-34, 142-144, 152-154, 162-164, 172-174, 182-184, 187-189, 192-194, 197-199, 202-204, 207-209, 212-214, 217-219, 222-224, 227-229, 232-234, or any variant thereof, and/or light chain CDRs selected from the amino acid sequences of SEQ ID NOs: 7-9, 17-19, 27-29, 37-39, 47-49, 57-59, 67-69, 77-79, 87-89, 97-99, 107-109, 117-119, 127-129, 137-139, 147-149, 157-159, 167-169, 177-179, 237-239, 242-244, 247-249, 252-254, 257-259, 262-264, 267-269, or any variant thereof.

2. The antibody or antigen-binding portion thereof according to claim 1, comprising a heavy chain CDR1 selected from the amino acid sequences of SEQ ID NOs: 2, 12, 22, 32, 42, 52, 62, 72, 82, 92, 102, 112, 122, 132, 142, 152, 162, 172, 182, 187, 192, 197, 202, 207, 212, 217, 222, 227, 232, or any variant thereof, a heavy chain CDR2 selected from the amino acid sequences of SEQ ID NOs: 3, 13, 23, 33, 43, 53, 63, 73, 83, 93, 103, 113, 123, 133, 143, 153, 163, 173, 183, 188, 193, 198, 203, 208, 213, 218, 223, 228, 233, or any variant thereof, and a heavy chain CDR3 selected from the amino acid sequences of SEQ ID NOs: 4, 14, 24, 34, 44, 54, 64, 74, 84, 94, 104, 114, 124, 134, 144, 154, 164, 174, 184, 189, 194, 199, 204, 209, 214, 219, 224, 229, 234, or any variant thereof and/or a light chain CDR1 selected from the amino acid sequences of SEQ ID NOs: 7, 17, 27, 37, 47, 57, 67, 77, 87, 97, 107, 117, 127, 137, 147, 157, 167, 177, 237, 242, 247, 252, 257, 262, 267, or any variant thereof, a light chain CDR2 selected from the amino acid sequences of SEQ ID NOs: 8, 18, 28, 38, 48, 58, 68, 78, 88, 98, 108, 118, 128, 138, 148, 158, 168, 178, 238, 243, 248, 253, 258, 263, 268, or any variant thereof, and a light chain CDR3 selected from the amino acid sequences of SEQ ID NOs: 9, 19, 29, 39, 49, 59, 69, 79, 89, 99, 109, 119, 129, 139, 149, 159, 169, 179, 239, 244, 249, 254, 259, 264, 269, or any variant thereof.

3. The antibody or antigen-binding portion thereof according to claim 1, comprising a CDR combination of heavy and light chains selected from the group consisting of:
(1) the heavy chain CDR1, CDR2, and CDR3 sequences including SEQ ID NOs: 2-4, respectively, and/or the light chain CDR1, CDR2, and CDR3 sequences including SEQ ID NOs: 7-9, respectively;
(2) the heavy chain CDR1, CDR2, and CDR3 sequences including SEQ ID NOs: 12-14, respectively, and/or the light chain CDR1, CDR2, and CDR3 sequences including SEQ ID NOs: 17-19, respectively;
(3) the heavy chain CDR1, CDR2, and CDR3 sequences including SEQ ID NOs: 22-24, respectively, and/or the light chain CDR1, CDR2, and CDR3 sequences including SEQ ID NOs: 27-29, respectively;
(4) the heavy chain CDR1, CDR2, and CDR3 sequences including SEQ ID NOs: 32-34, respectively, and/or the light chain CDR1, CDR2, and CDR3 sequences including SEQ ID NOs: 37-39, respectively;
(5) the heavy chain CDR1, CDR2, and CDR3 sequences including SEQ ID NOs: 42-44, respectively, and/or the light chain CDR1, CDR2, and CDR3 sequences including SEQ ID NOs: 47-49, respectively;
(6) the heavy chain CDR1, CDR2, and CDR3 sequences including SEQ ID NOs: 52-54, respectively, and/or the light chain CDR1, CDR2, and CDR3 sequences including SEQ ID NOs: 57-59, respectively;
(7) the heavy chain CDR1, CDR2, and CDR3 sequences including SEQ ID NOs: 62-64, respectively, and/or the light chain CDR1, CDR2, and CDR3 sequences including SEQ ID NOs: 67-69, respectively;
(8) the heavy chain CDR1, CDR2, and CDR3 sequences including SEQ ID NOs: 72-74, respectively, and/or the light chain CDR1, CDR2, and CDR3 sequences including SEQ ID NOs: 77-79, respectively;
(9) the heavy chain CDR1, CDR2, and CDR3 sequences including SEQ ID NOs: 82-84, respectively, and/or the light chain CDR1, CDR2, and CDR3 sequences including SEQ ID NOs: 87-89, respectively;
(10) the heavy chain CDR1, CDR2, and CDR3 sequences including SEQ ID NOs: 92-94, respectively, and/or the light chain CDR1, CDR2, and CDR3 sequences including SEQ ID NOs: 97-99, respectively;
(11) the heavy chain CDR1, CDR2, and CDR3 sequences including SEQ ID NOs: 102-104, respectively, and/or the light chain CDR1, CDR2, and CDR3 sequences including SEQ ID NOs: 107-109, respectively;
(12) the heavy chain CDR1, CDR2, and CDR3 sequences including SEQ ID NOs: 112-114, respectively, and/or the light chain CDR1, CDR2, and CDR3 sequences including SEQ ID NOs: 117-119, respectively;
(13) the heavy chain CDR1, CDR2, and CDR3 sequences including SEQ ID NOs: 122-124, respectively, and/or the light chain CDR1, CDR2, and CDR3 sequences including SEQ ID NOs: 127-129, respectively;
(14) the heavy chain CDR1, CDR2, and CDR3 sequences including SEQ ID NOs: 132-134, respectively, and/or the light chain CDR1, CDR2, and CDR3 sequences including SEQ ID NOs: 137-139, respectively;
(15) the heavy chain CDR1, CDR2, and CDR3 sequences including SEQ ID NOs: 142-144, respectively, and/or the light chain CDR1, CDR2, and CDR3 sequences including SEQ ID NOs: 147-149, respectively;
(16) the heavy chain CDR1, CDR2, and CDR3 sequences including SEQ ID NOs: 152-154, respectively, and/or the light chain CDR1, CDR2, and CDR3 sequences including SEQ ID NOs: 157-159, respectively;
(17) the heavy chain CDR1, CDR2, and CDR3 sequences including SEQ ID NOs: 162-164, respectively, and/or the light chain CDR1, CDR2, and CDR3 sequences including SEQ ID NOs: 167-169, respectively;
(18) the heavy chain CDR1, CDR2, and CDR3 sequences including SEQ ID NOs: 172-174, respectively, and/or the light chain CDR1, CDR2, and CDR3 sequences including SEQ ID NOs: 177-179, respectively;
(19) the heavy chain CDR1, CDR2, and CDR3 sequences including SEQ ID NOs: 182-184, respectively, and/or the light chain CDR1, CDR2, and CDR3 sequences including SEQ ID NOs: 237-239, respectively;
(20) the heavy chain CDR1, CDR2, and CDR3 sequences including SEQ ID NOs: 182-184, respectively, and/or the light chain CDR1, CDR2, and CDR3 sequences including SEQ ID NOs: 242-244, respectively;
(21) the heavy chain CDR1, CDR2, and CDR3 sequences including SEQ ID NOs: 182-184, respectively, and/or the light chain CDR1, CDR2, and CDR3 sequences including SEQ ID NOs: 247-249, respectively;
(22) the heavy chain CDR1, CDR2, and CDR3 sequences including SEQ ID NOs: 182-184, respectively, and/or the light chain CDR1, CDR2, and CDR3 sequences including SEQ ID NOs: 252-254, respectively;
(23) the heavy chain CDR1, CDR2, and CDR3 sequences including SEQ ID NOs: 182-184, respectively, and/or the light chain CDR1, CDR2, and CDR3 sequences including SEQ ID NOs: 257-259, respectively;
(24) the heavy chain CDR1, CDR2, and CDR3 sequences including SEQ ID NOs: 187-189, respectively, and/or the light chain CDR1, CDR2, and CDR3 sequences including SEQ ID NOs: 237-239, respectively;
(25) the heavy chain CDR1, CDR2, and CDR3 sequences including SEQ ID NOs: 187-189, respectively, and/or the light chain CDR1, CDR2, and CDR3 sequences including SEQ ID NOs: 242-244, respectively;
(26) the heavy chain CDR1, CDR2, and CDR3 sequences including SEQ ID NOs: 187-189, respectively, and/or the light chain CDR1, CDR2, and CDR3 sequences including SEQ ID NOs: 247-249, respectively;
(27) the heavy chain CDR1, CDR2, and CDR3 sequences including SEQ ID NOs: 187-189, respectively, and/or the light chain CDR1, CDR2, and CDR3 sequences including SEQ ID NOs: 252-254, respectively;
(28) the heavy chain CDR1, CDR2, and CDR3 sequences including SEQ ID NOs: 187-189, respectively, and/or the light chain CDR1, CDR2, and CDR3 sequences including SEQ ID NOs: 257-259, respectively;
(29) the heavy chain CDR1, CDR2, and CDR3 sequences including SEQ ID NOs: 192-194, respectively, and/or the light chain CDR1, CDR2, and CDR3 sequences including SEQ ID NOs: 237-239, respectively;
(30) the heavy chain CDR1, CDR2, and CDR3 sequences including SEQ ID NOs: 192-194, respectively, and/or the light chain CDR1, CDR2, and CDR3 sequences including SEQ ID NOs: 242-244, respectively;
(31) the heavy chain CDR1, CDR2, and CDR3 sequences including SEQ ID NOs: 192-194, respectively, and/or the light chain CDR1, CDR2, and CDR3 sequences including SEQ ID NOs: 247-249, respectively;
(32) the heavy chain CDR1, CDR2, and CDR3 sequences including SEQ ID NOs: 192-194, respectively, and/or the light chain CDR1, CDR2, and CDR3 sequences including SEQ ID NOs: 252-254, respectively;
(33) the heavy chain CDR1, CDR2, and CDR3 sequences including SEQ ID NOs: 192-194, respectively, and/or the light chain CDR1, CDR2, and CDR3 sequences including SEQ ID NOs: 257-259, respectively;
(34) the heavy chain CDR1, CDR2, and CDR3 sequences including SEQ ID NOs: 197-199, respectively, and/or the light chain CDR1, CDR2, and CDR3 sequences including SEQ ID NOs: 237-239, respectively;
(35) the heavy chain CDR1, CDR2, and CDR3 sequences including SEQ ID NOs: 197-199, respectively, and/or the light chain CDR1, CDR2, and CDR3 sequences including SEQ ID NOs: 242-244, respectively;
(36) the heavy chain CDR1, CDR2, and CDR3 sequences including SEQ ID NOs: 197-199, respectively, and/or the light chain CDR1, CDR2, and CDR3 sequences including SEQ ID NOs: 247-249, respectively;
(37) the heavy chain CDR1, CDR2, and CDR3 sequences including SEQ ID NOs: 197-199, respectively, and/or the light chain CDR1, CDR2, and CDR3 sequences including SEQ ID NOs: 252-254, respectively;
(38) the heavy chain CDR1, CDR2, and CDR3 sequences including SEQ ID NOs: 197-199, respectively, and/or the light chain CDR1, CDR2, and CDR3 sequences including SEQ ID NOs: 257-259, respectively;
(39) the heavy chain CDR1, CDR2, and CDR3 sequences including SEQ ID NOs: 202-204, respectively, and/or the light chain CDR1, CDR2, and CDR3 sequences including SEQ ID NOs: 237-239, respectively;
(40) the heavy chain CDR1, CDR2, and CDR3 sequences including SEQ ID NOs: 202-204, respectively, and/or the light chain CDR1, CDR2, and CDR3 sequences including SEQ ID NOs: 242-244, respectively;
(41) the heavy chain CDR1, CDR2, and CDR3 sequences including SEQ ID NOs: 202-204, respectively, and/or the light chain CDR1, CDR2, and CDR3 sequences including SEQ ID NOs: 247-249, respectively;
(42) the heavy chain CDR1, CDR2, and CDR3 sequences including SEQ ID NOs: 202-204, respectively, and/or the light chain CDR1, CDR2, and CDR3 sequences including SEQ ID NOs: 252-254, respectively;
(43) the heavy chain CDR1, CDR2, and CDR3 sequences including SEQ ID NOs: 202-204, respectively, and/or the light chain CDR1, CDR2, and CDR3 sequences including SEQ ID NOs: 257-259, respectively;
(44) the heavy chain CDR1, CDR2, and CDR3 sequences including SEQ ID NOs: 207-209, respectively, and/or the light chain CDR1, CDR2, and CDR3 sequences including SEQ ID NOs: 237-239, respectively;
(45) the heavy chain CDR1, CDR2, and CDR3 sequences including SEQ ID NOs: 207-209, respectively, and/or the light chain CDR1, CDR2, and CDR3 sequences including SEQ ID NOs: 242-244, respectively;
(46) the heavy chain CDR1, CDR2, and CDR3 sequences including SEQ ID NOs: 207-209, respectively, and/or the light chain CDR1, CDR2, and CDR3 sequences including SEQ ID NOs: 247-249, respectively;
(47) the heavy chain CDR1, CDR2, and CDR3 sequences including SEQ ID NOs: 207-209, respectively, and/or the light chain CDR1, CDR2, and CDR3 sequences including SEQ ID NOs: 252-254, respectively;
(48) the heavy chain CDR1, CDR2, and CDR3 sequences including SEQ ID NOs: 207-209, respectively, and/or the light chain CDR1, CDR2, and CDR3 sequences including SEQ ID NOs: 257-259, respectively;
(49) the heavy chain CDR1, CDR2, and CDR3 sequences including SEQ ID NOs: 212-214, respectively, and/or the light chain CDR1, CDR2, and CDR3 sequences including SEQ ID NOs: 262-264, respectively;
(50) the heavy chain CDR1, CDR2, and CDR3 sequences including SEQ ID NOs: 212-214, respectively, and/or the light chain CDR1, CDR2, and CDR3 sequences including SEQ ID NOs: 267-269, respectively;
(51) the heavy chain CDR1, CDR2, and CDR3 sequences including SEQ ID NOs: 217-219, respectively, and/or the light chain CDR1, CDR2, and CDR3 sequences including SEQ ID NOs: 262-264, respectively;
(52) the heavy chain CDR1, CDR2, and CDR3 sequences including SEQ ID NOs: 217-219, respectively, and/or the light chain CDR1, CDR2, and CDR3 sequences including SEQ ID NOs: 267-269, respectively;
(53) the heavy chain CDR1, CDR2, and CDR3 sequences including SEQ ID NOs: 222-224, respectively, and/or the light chain CDR1, CDR2, and CDR3 sequences including SEQ ID NOs: 262-264, respectively;
(54) the heavy chain CDR1, CDR2, and CDR3 sequences including SEQ ID NOs: 222-224, respectively, and/or the light chain CDR1, CDR2, and CDR3 sequences including SEQ ID NOs: 267-269, respectively;
(55) the heavy chain CDR1, CDR2, and CDR3 sequences including SEQ ID NOs: 227-229, respectively, and/or the light chain CDR1, CDR2, and CDR3 sequences including SEQ ID NOs: 262-264, respectively;
(56) the heavy chain CDR1, CDR2, and CDR3 sequences including SEQ ID NOs: 227-229, respectively, and/or the light chain CDR1, CDR2, and CDR3 sequences including SEQ ID NOs: 267-269, respectively;
(57) the heavy chain CDR1, CDR2, and CDR3 sequences including SEQ ID NOs: 232-234, respectively, and/or the light chain CDR1, CDR2, and CDR3 sequences including SEQ ID NOs: 262-264, respectively;
(58) the heavy chain CDR1, CDR2, and CDR3 sequences including SEQ ID NOs: 232-234, respectively, and/or the light chain CDR1, CDR2, and CDR3 sequences including SEQ ID NOs: 267-269, respectively.

4. The antibody or antigen-binding portion thereof according to claim 1, comprising a heavy chain variable region selected from the amino acid sequences of SEQ ID NOs: 1, 11, 21, 31, 41, 51, 61, 71, 81, 91, 101, 111, 121, 131, 141, 151, 161, 171, 181, 186, 191, 196, 201, 206, 211, 216, 221, 226, 231, or any variant thereof, and/or a light chain variable region selected from the amino acid sequence of SEQ ID NOs: 6, 16, 26, 36, 46, 56, 66, 76, 86, 96, 106, 116, 126, 136, 146, 156, 166, 176, 236, 241, 246, 251, 256, 261, 266, or any variant thereof;
preferably, comprising a heavy chain variable region of the amino acid sequence of SEQ ID NO: 1 or any variant thereof, and a light chain variable region of the amino acid sequence of SEQ ID NO: 6 or any variant thereof;
preferably, comprising a heavy chain variable region of the amino acid sequence of SEQ ID NO: 11 or any variant thereof, and a light chain variable region of the amino acid sequence of SEQ ID NO: 16 or any variant thereof;
preferably, comprising a heavy chain variable region of the amino acid sequence of SEQ ID NO: 21 or any variant thereof, and a light chain variable region of the amino acid sequence of SEQ ID NO: 26 or any variant thereof;
preferably, comprising a heavy chain variable region of the amino acid sequence of SEQ ID NO: 31 or any variant thereof, and a light chain variable region of the amino acid sequence of SEQ ID NO: 36 or any variant thereof;
preferably, comprising a heavy chain variable region of the amino acid sequence of SEQ ID NO: 41 or any variant thereof, and a light chain variable region of the amino acid sequence of SEQ ID NO: 46 or any variant thereof;
preferably, comprising a heavy chain variable region of the amino acid sequence of SEQ ID NO: 51 or any variant thereof, and a light chain variable region of the amino acid sequence of SEQ ID NO: 56 or any variant thereof;
preferably, comprising a heavy chain variable region of the amino acid sequence of SEQ ID NO: 61 or any variant thereof, and a light chain variable region of the amino acid sequence of SEQ ID NO: 66 or any variant thereof;
preferably, comprising a heavy chain variable region of the amino acid sequence of SEQ ID NO: 71 or any variant thereof, and a light chain variable region of the amino acid sequence of SEQ ID NO: 76 or any variant thereof;
preferably, comprising a heavy chain variable region of the amino acid sequence of SEQ ID NO: 81 or any variant thereof, and a light chain variable region of the amino acid sequence of SEQ ID NO: 86 or any variant thereof;
preferably, comprising a heavy chain variable region of the amino acid sequence of SEQ ID NO: 91 or any variant thereof, and a light chain variable region of the amino acid sequence of SEQ ID NO: 96 or any variant thereof;
preferably, comprising a heavy chain variable region of the amino acid sequence of SEQ ID NO: 101 or any variant thereof, and a light chain variable region of the amino acid sequence of SEQ ID NO: 106 or any variant thereof;
preferably, comprising a heavy chain variable region of the amino acid sequence of SEQ ID NO: 111 or any variant thereof, and a light chain variable region of the amino acid sequence of SEQ ID NO: 116 or any variant thereof;
preferably, comprising a heavy chain variable region of the amino acid sequence of SEQ ID NO: 121 or any variant thereof, and a light chain variable region of the amino acid sequence of SEQ ID NO: 126 or any variant thereof;
preferably, comprising a heavy chain variable region of the amino acid sequence of SEQ ID NO: 131 or any variant thereof, and a light chain variable region of the amino acid sequence of SEQ ID NO: 136 or any variant thereof;
preferably, comprising a heavy chain variable region of the amino acid sequence of SEQ ID NO: 141 or any variant thereof, and a light chain variable region of the amino acid sequence of SEQ ID NO: 146 or any variant thereof;
preferably, comprising a heavy chain variable region of the amino acid sequence of SEQ ID NO: 151 or any variant thereof, and a light chain variable region of the amino acid sequence of SEQ ID NO: 156 or any variant thereof;
preferably, comprising a heavy chain variable region of the amino acid sequence of SEQ ID NO: 161 or any variant thereof, and a light chain variable region of the amino acid sequence of SEQ ID NO: 166 or any variant thereof;
preferably, comprising a heavy chain variable region of the amino acid sequence of SEQ ID NO: 171 or any variant thereof, and a light chain variable region of the amino acid sequence of SEQ ID NO: 176 or any variant thereof;
preferably, comprising a heavy chain variable region of the amino acid sequence of SEQ ID NO: 181 or any variant thereof, and a light chain variable region of the amino acid sequence of SEQ ID NO: 236 or any variant thereof;
preferably, comprising a heavy chain variable region of the amino acid sequence of SEQ ID NO: 181 or any variant thereof, and a light chain variable region of the amino acid sequence of SEQ ID NO: 241 or any variant thereof;
preferably, comprising a heavy chain variable region of the amino acid sequence of SEQ ID NO: 181 or any variant thereof, and a light chain variable region of the amino acid sequence of SEQ ID NO: 246 or any variant thereof;
preferably, comprising a heavy chain variable region of the amino acid sequence of SEQ ID NO: 181 or any variant thereof, and a light chain variable region of the amino acid sequence of SEQ ID NO: 251 or any variant thereof;
preferably, comprising a heavy chain variable region of the amino acid sequence of SEQ ID NO: 181 or any variant thereof, and a light chain variable region of the amino acid sequence of SEQ ID NO: 256 or any variant thereof;
preferably, comprising a heavy chain variable region of the amino acid sequence of SEQ ID NO: 186 or any variant thereof, and a light chain variable region of the amino acid sequence of SEQ ID NO: 236 or any variant thereof;
preferably, comprising a heavy chain variable region of the amino acid sequence of SEQ ID NO: 186 or any variant thereof, and a light chain variable region of the amino acid sequence of SEQ ID NO: 241 or any variant thereof;
preferably, comprising a heavy chain variable region of the amino acid sequence of SEQ ID NO: 186 or any variant thereof, and a light chain variable region of the amino acid sequence of SEQ ID NO: 246 or any variant thereof;
preferably, comprising a heavy chain variable region of the amino acid sequence of SEQ ID NO: 186 or any variant thereof, and a light chain variable region of the amino acid sequence of SEQ ID NO: 251 or any variant thereof;
preferably, comprising a heavy chain variable region of the amino acid sequence of SEQ ID NO: 186 or any variant thereof, and a light chain variable region of the amino acid sequence of SEQ ID NO: 256 or any variant thereof;
preferably, comprising a heavy chain variable region of the amino acid sequence of SEQ ID NO: 191 or any variant thereof, and a light chain variable region of the amino acid sequence of SEQ ID NO: 236 or any variant thereof;
preferably, comprising a heavy chain variable region of the amino acid sequence of SEQ ID NO: 191 or any variant thereof, and a light chain variable region of the amino acid sequence of SEQ ID NO: 241 or any variant thereof;
preferably, comprising a heavy chain variable region of the amino acid sequence of SEQ ID NO: 191 or any variant thereof, and a light chain variable region of the amino acid sequence of SEQ ID NO: 246 or any variant thereof;
preferably, comprising a heavy chain variable region of the amino acid sequence of SEQ ID NO: 191 or any variant thereof, and a light chain variable region of the amino acid sequence of SEQ ID NO: 251 or any variant thereof;
preferably, comprising a heavy chain variable region of the amino acid sequence of SEQ ID NO: 191 or any variant thereof, and a light chain variable region of the amino acid sequence of SEQ ID NO: 256 or any variant thereof;
preferably, comprising a heavy chain variable region of the amino acid sequence of SEQ ID NO: 196 or any variant thereof, and a light chain variable region of the amino acid sequence of SEQ ID NO: 236 or any variant thereof;
preferably, comprising a heavy chain variable region of the amino acid sequence of SEQ ID NO: 196 or any variant thereof, and a light chain variable region of the amino acid sequence of SEQ ID NO: 241 or any variant thereof;
preferably, comprising a heavy chain variable region of the amino acid sequence of SEQ ID NO: 196 or any variant thereof, and a light chain variable region of the amino acid sequence of SEQ ID NO: 246 or any variant thereof;
preferably, comprising a heavy chain variable region of the amino acid sequence of SEQ ID NO: 196 or any variant thereof, and a light chain variable region of the amino acid sequence of SEQ ID NO: 251 or any variant thereof;
preferably, comprising a heavy chain variable region of the amino acid sequence of SEQ ID NO: 196 or any variant thereof, and a light chain variable region of the amino acid sequence of SEQ ID NO: 256 or any variant thereof;
preferably, comprising a heavy chain variable region of the amino acid sequence of SEQ ID NO: 201 or any variant thereof, and a light chain variable region of the amino acid sequence of SEQ ID NO: 236 or any variant thereof;
preferably, comprising a heavy chain variable region of the amino acid sequence of SEQ ID NO: 201 or any variant thereof, and a light chain variable region of the amino acid sequence of SEQ ID NO: 241 or any variant thereof;
preferably, comprising a heavy chain variable region of the amino acid sequence of SEQ ID NO: 201 or any variant thereof, and a light chain variable region of the amino acid sequence of SEQ ID NO: 246 or any variant thereof;
preferably, comprising a heavy chain variable region of the amino acid sequence of SEQ ID NO: 201 or any variant thereof, and a light chain variable region of the amino acid sequence of SEQ ID NO: 251 or any variant thereof;
preferably, comprising a heavy chain variable region of the amino acid sequence of SEQ ID NO: 201 or any variant thereof, and a light chain variable region of the amino acid sequence of SEQ ID NO: 256 or any variant thereof;
preferably, comprising a heavy chain variable region of the amino acid sequence of SEQ ID NO: 206 or any variant thereof, and a light chain variable region of the amino acid sequence of SEQ ID NO: 236 or any variant thereof;
preferably, comprising a heavy chain variable region of the amino acid sequence of SEQ ID NO: 206 or any variant thereof, and a light chain variable region of the amino acid sequence of SEQ ID NO: 241 or any variant thereof;
preferably, comprising a heavy chain variable region of the amino acid sequence of SEQ ID NO: 206 or any variant thereof, and a light chain variable region of the amino acid sequence of SEQ ID NO: 246 or any variant thereof;
preferably, comprising a heavy chain variable region of the amino acid sequence of SEQ ID NO: 206 or any variant thereof, and a light chain variable region of the amino acid sequence of SEQ ID NO: 251 or any variant thereof;
preferably, comprising a heavy chain variable region of the amino acid sequence of SEQ ID NO: 206 or any variant thereof, and a light chain variable region of the amino acid sequence of SEQ ID NO: 256 or any variant thereof;
preferably, comprising a heavy chain variable region of the amino acid sequence of SEQ ID NO: 211 or any variant thereof, and a light chain variable region of the amino acid sequence of SEQ ID NO: 261 or any variant thereof;
preferably, comprising a heavy chain variable region of the amino acid sequence of SEQ ID NO: 211 or any variant thereof, and a light chain variable region of the amino acid sequence of SEQ ID NO: 266 or any variant thereof;
preferably, comprising a heavy chain variable region of the amino acid sequence of SEQ ID NO: 216 or any variant thereof, and a light chain variable region of the amino acid sequence of SEQ ID NO: 261 or any variant thereof;
preferably, comprising a heavy chain variable region of the amino acid sequence of SEQ ID NO: 216 or any variant thereof, and a light chain variable region of the amino acid sequence of SEQ ID NO: 266 or any variant thereof;
preferably, comprising a heavy chain variable region of the amino acid sequence of SEQ ID NO: 221 or any variant thereof, and a light chain variable region of the amino acid sequence of SEQ ID NO: 261 or any variant thereof;
preferably, comprising a heavy chain variable region of the amino acid sequence of SEQ ID NO: 221 or any variant thereof, and a light chain variable region of the amino acid sequence of SEQ ID NO: 266 or any variant thereof;
preferably, comprising a heavy chain variable region of the amino acid sequence of SEQ ID NO: 226 or any variant thereof, and a light chain variable region of the amino acid sequence of SEQ ID NO: 261 or any variant thereof;
preferably, comprising a heavy chain variable region of the amino acid sequence of SEQ ID NO: 226 or any variant thereof, and a light chain variable region of the amino acid sequence of SEQ ID NO: 266 or any variant thereof;
preferably, comprising a heavy chain variable region of the amino acid sequence of SEQ ID NO: 231 or any variant thereof, and a light chain variable region of the amino acid sequence of SEQ ID NO: 261 or any variant thereof;
preferably, comprising a heavy chain variable region of the amino acid sequence of SEQ ID NO: 231 or any variant thereof, and a light chain variable region of the amino acid sequence of SEQ ID NO: 266 or any variant thereof.

5. A bispecific or multispecific molecule, comprising the antibody or antigen-binding portion thereof according to claim 1.

6. The antibody or antigen-binding portion thereof according to claim 1 or a bispecific or multispecific molecule comprising the antibody or antigen-binding portion thereof, which is humanized.

7. A nucleic acid molecule encoding the antibody or antigen-binding portion thereof according to claim 1 or encoding a bispecific or multispecific molecule comprising the antibody or antigen-binding portion thereof; preferably, the nucleic acid molecule comprises the nucleotide sequence of the antibody heavy chain selected from SEQ ID NOs: 5, 15, 25, 35, 45, 55, 65, 75, 85, 95, 105, 115, 125, 135, 145, 155, 165, 175, 185, 190, 195, 200, 205, 210, 215, 220, 225, 230, 235, or any variant thereof, and/or the nucleotide sequence of the antibody light chain selected from SEQ ID NOs: 10, 20, 30, 40, 50, 60, 70, 80, 90, 100, 110, 120, 130, 140, 150, 160, 170, 180, 240, 245, 250, 255, 260, 265, 270, or any variant thereof.

8. A vector comprising the nucleic acid according to claim 7.

9. A cell containing the nucleic acid according to claim 7 or a vector comprising the nucleic acid molecule.

10. A composition comprising the antibody or antigen-binding portion thereof according to claim 1, a bispecific or multispecific molecule comprising the antibody or antigen-binding portion thereof, a nucleic acid molecule encoding the antibody or antigen-binding portion thereof, a vector comprising the nucleic acid molecule and/or a cell containing the nucleic acid molecule or the vector.

11. A kit comprising the antibody or antigen-binding portion thereof according to claim 1, a bispecific or multispecific molecule comprising the antibody or antigen-binding portion thereof, a nucleic acid molecule encoding the antibody or antigen-binding portion thereof, a vector comprising the nucleic acid molecule, a cell containing the nucleic acid molecule or the vector and/or a composition comprising the antibody or antigen-binding portion thereof, the nucleic acid molecule, the vector and/or the cell.

12. (canceled)

13. A method of treating a CD38-related disorder, comprising the step of administering to the mammal a therapeutically effective amount of the antibody or antigen-binding portion thereof according to claim 1, a bispecific or multispecific molecule comprising the antibody or antigen-binding portion thereof, a nucleic acid molecule encoding the antibody or antigen-binding portion thereof, a vector comprising the nucleic acid molecule, a cell containing the nucleic acid molecule or the vector and/or a composition comprising the antibody or antigen-binding portion thereof, the nucleic acid molecule, the vector and/or the cell, preferably, the CD38-related disorder is selected from tumors and autoimmune diseases; preferably, the tumor is selected from the group consisting of multiple myeloma (MM), NK/T cell lymphoma, immunoglobulin light chain amyloidosis, chronic lymphocytic leukemia (CLL), chronic myelogenous leukemia (CML), T cell acute lymphocytic leukemia, acute myelogenous leukemia (AML) and acute lymphocytic leukemia (ALL); preferably, the autoimmune disease is selected from rheumatoid arthritis (RA) or systemic lupus erythematosus (SLE).


SEQUENCE LISTING (270 sequences)

SEQ ID NO: 1 - protein, 114 aa, Mus musculus
QIQLVQSGPELKKPGETVKISCKASGYTFTNHGMNWVKQAPGKGLKWMGWINTYTGEPTYGEDFKGRFAFSLETSASTAYLQINNLKNEDLATYFCARKGFVYWGQGTLVTVSV

SEQ ID NO: 2 - protein, 6 aa, Mus musculus
TNHGMN

SEQ ID NO: 3 - protein, 17 aa, Mus musculus
WINTYTGEPTYGEDFKG

SEQ ID NO: 4 - protein, 5 aa, Mus musculus
KGFVY

SEQ ID NO: 5 - DNA, 342 bp, Mus musculus
cagatccagttggtgcagtctggacctgagctgaagaagcctggagagacagtcaagatctcctgcaaggcttctgggtatactttcacaaaccatggaatgaactgggtgaagcaggctccaggaaagggcttaaagtggatgggctggataaacacctacactggagagccaacatatggtgaagacttcaagggacggtttgccttctctttggaaacctctgccagcactgcctatttgcagatcaacaacctcaaaaatgaggacctggctacatatttctgtgcaagaaaggggtttgtttactggggccaagggactctggtcactgtctctgta

SEQ ID NO: 6 - protein, 111 aa, Mus musculus
DIVLTQSPASLAVSLGQRATMSCRASESVDIYGNSFIYWYQQKPGQPPKLLIYRASILESGIPARFSGSGSRTDFTLTINPVEADDVATYYCQQINEDPFTFGGGTKLEIK

SEQ ID NO: 7 - protein, 15 aa, Mus musculus
RASESVDIYGNSFIY

SEQ ID NO: 8 - protein, 7 aa, Mus musculus
RASILES

SEQ ID NO: 9 - protein, 9 aa, Mus musculus
QQINEDPFT

SEQ ID NO: 10 - DNA, 332 bp, Mus musculus
acattgtgctgacccaatctccagcttctttggctgtgtctctagggcagagggccaccatgtcctgcagagccagtgaaagtgttgatatttatggcaatagttttatatactggtaccaacagaaaccaggacagccacccaaactcctcatctatcgtgcatccatcctagaatctgggatccctgccaggttcagtggcagtgggtctaggacagacttcaccctcaccattaatcctgtggaggctgatgatgttgcaacctattactgtcagcaaattaatgaagatccattcacgttcggaggggggaccaagctggaaataaaa

SEQ ID NO: 11 - protein, 120 aa, Mus musculus
QAYLQQSGAELVRSGASVKMSCKASGYTFTSYNIHWVKQTPGQGLKWIGYIYPGNGGTNYNQKFKVKATLTADTSSSTAYMQISSLTSEDSAVYFCARRGSDYDGGFASWGQGTLVTVSA

SEQ ID NO: 12 - protein, 6 aa, Mus musculus
TSYNIH

SEQ ID NO: 13 - protein, 17 aa, Mus musculus
YIYPGNGGTNYNQKFKV

SEQ ID NO: 14 - protein, 11 aa, Mus musculus
RGSDYDGGFAS

SEQ ID NO: 15 - DNA, 360 bp, Mus musculus
caggcttatctacagcagtctggggctgagctggtgaggtctggggcctcagtgaagatgtcctgcaaggcttctggctacacatttaccagttacaatatacactgggtaaagcagacacctggacagggcctgaaatggattggatatatctatcctggaaatggtggtactaactacaatcagaagttcaaggtcaaggccacattgactgcagacacatcctccagcacagcctacatgcagatcagcagcctgacatctgaagactctgcggtctatttctgtgcaagaagggggtctgattacgacggggggtttgcttcctggggccaagggactctggtcactgtctctgca

SEQ ID NO: 16 - protein, 111 aa, Mus musculus
DILLTQSPASLAVSLGQRATISCRASKSVDIYGNSFIHWYQQKPGQSPKLLIYLASNLDSGVPARFSGSGSRTDFTLTIDPVEADDAATYYCQQNNEDPLTFGAGTKLELK

SEQ ID NO: 17 - protein, 15 aa, Mus musculus
RASKSVDIYGNSFIH

SEQ ID NO: 18 - protein, 7 aa, Mus musculus
LASNLDS

SEQ ID NO: 19 - protein, 9 aa, Mus musculus
QQNNEDPLT

SEQ ID NO: 20 - DNA, 333 bp, Mus musculus
gacattttgctgacccagtctccagcttctttggctgtgtctctagggcagagggccaccatatcctgcagagccagtaaaagtgttgatatttatggcaatagttttatacactggtaccagcagaaaccaggacagtcacccaaactcctcatctatcttgcatccaacctagattctggggtccctgccaggttcagtggcagtgggtctaggacagacttcaccctcaccattgatcctgtggaggctgatgatgctgcaacctattactgtcagcaaaataatgaggatccgctcacgttcggtgctgggaccaagctggagctgaaa

SEQ ID NO: 21 - protein, 119 aa, Mus musculus
QLQLKQSGPGLVQPSQSLSITCTVSGFSLTSFGVHWIRQSPGKGLEWLGVIWRGGSTDYNAAFMSRLSITKDNSKSQVFFKMNSLQADDTAIYYCAKTMITRGYTMDYWGQGTSVTVSS

SEQ ID NO: 22 - protein, 6 aa, Mus musculus
TSFGVH

SEQ ID NO: 23 - protein, 16 aa, Mus musculus
VIWRGGSTDYNAAFMS

SEQ ID NO: 24 - protein, 11 aa, Mus musculus
TMITRGYTMDY

SEQ ID NO: 25 - DNA, 357 bp, Mus musculus
cagttgcagctgaagcagtcaggacctggcctagtgcagccctcacagagcctgtccataacctgcacagtctctggtttctcattaactagctttggtgtacactggattcgccagtctccaggaaagggtctggagtggctgggagtgatttggagaggtggaagcacagactacaatgcagctttcatgtccagactgagcatcaccaaggacaactccaagagccaagttttctttaaaatgaacagtctgcaagctgatgacactgccatatattactgtgccaaaactatgattacgagggggtatactatggactactggggtcaaggaacctcagtcaccgtctcctca

SEQ ID NO: 26 - protein, 107 aa, Mus musculus
DIQMTQSSSSFSVSLGDRVTITCKASEDIYNRLAWYQQKPGNAPRLLISGATTLETGVPSRFSGSGSGKDYTLSITSLQTEDVATYYCQQFWSTPYTFGGGTKLEMI

SEQ ID NO: 27 - protein, 11 aa, Mus musculus
KASEDIYNRLA

SEQ ID NO: 28 - protein, 7 aa, Mus musculus
GATTLET

SEQ ID NO: 29 - protein, 9 aa, Mus musculus
QQFWSTPYT

SEQ ID NO: 30 - DNA, 321 bp, Mus musculus
gacatccagatgacacaatcctcatcctccttttctgtatctctaggagacagggtcaccattacttgcaaggcaagtgaggacatatataatcggttagcctggtatcagcagaaaccaggaaatgctcctaggctcttaatatctggtgcaaccactttggaaactggggttccttcaagattcagtggcagtggatctggaaaggattacactctcagcattaccagtcttcagactgaagatgttgctacttattactgtcaacagttttggagtactccgtacacgttcggaggggggaccaagctggaaatgata

SEQ ID NO: 31 - protein, 118 aa, Mus musculus
EVQLVESGGGLVKPGGSLKLSCEGSGFTFNNYAMSWVRQSPEKRLEWVAEISTGGTFTYYVDTVTGRFTISRDNAKNTLYLEMSSLRSEDTARYYCARGYRYDAADYWGQGISVTVSS

SEQ ID NO: 32 - protein, 6 aa, Mus musculus
NNYAMS

SEQ ID NO: 33 - protein, 17 aa, Mus musculus
EISTGGTFTYYVDTVTG

SEQ ID NO: 34 - protein, 9 aa, Mus musculus
GYRYDAADY

SEQ ID NO: 35 - DNA, 354 bp, Mus musculus
gaagtgcagctggtggagtctgggggaggcttagtgaagcctggagggtccctgaaactctcctgtgaaggctctggattcactttcaacaactatgccatgtcttgggttcgccagtctccagagaagaggctggagtgggtcgcagagatttctactggtggtactttcacctactatgtagacactgtgacgggccgattcaccatctccagagacaatgccaagaacaccctgtacctggaaatgagtagtctgaggtctgaggacacggccaggtattattgtgcaagaggctataggtacgacgcggcggactactggggtcaaggaatctcagtcaccgtctcctca

SEQ ID NO: 36 - protein, 106 aa, Mus musculus
DIQMTQSPSSLSASLGERVSLTCRASQEISGYLIWLQQKPDGTIKRLIYATSTLDSGVPKRFSGSRSGSDYTLTISSLESEDFADYYCLQYANYFTFGAGTKLELK

SEQ ID NO: 37 - protein, 11 aa, Mus musculus
RASQEISGYLI

SEQ ID NO: 38 - protein, 7 aa, Mus musculus
ATSTLDS

SEQ ID NO: 39 - protein, 8 aa, Mus musculus
LQYANYFT

SEQ ID NO: 40 - DNA, 318 bp, Mus musculus
gacatccagatgacccagtccccctcctccctctccgcctccctcggcgagcgcgtctccctcacctgccgcgcctcccaggagatctccggctacctcatctggctccagcagaagcccgacggcaccatcaagcgcctcatctacgccacctccaccctcgactccggcgtccccaagcgcttctccggctcccgctccggctccgactacaccctcaccatctcctccctcgagtccgaggacttcgccgactactactgcctccagtacgccaactacttcaccttcggcgccggcaccaagctcgagctcaag

SEQ ID NO: 41 - protein, 119 aa, Mus musculus
QVQLNQSGPGLVQPSQSLSITCTVSGFSLTSYGVHWLRQSAGKGLEWLGVIWRGGSTDYNVGFMSRLTISKDNSKSQVFFKMNSLQGDDTAIYFCAKSMITTGYTMDYWGQGTSVTVSS

SEQ ID NO: 42 - protein, 6 aa, Mus musculus
TSYGVH

SEQ ID NO: 43 - protein, 16 aa, Mus musculus
VIWRGGSTDYNVGFMS

SEQ ID NO: 44 - protein, 11 aa, Mus musculus
SMITTGYTMDY

SEQ ID NO: 45 - DNA, 357 bp, Mus musculus
caggtgcagctgaaccagtcaggacctggcctagtgcagccctcacagagcctgtccataacctgcacagtctctggtttctcattaactagctatggtgtacattggcttcgccagtctgcaggaaagggtctggagtggctgggagtgatatggagaggtggaagcacagactacaatgtaggtttcatgtccagactgaccattagtaaggacaattccaagagccaagttttctttaaaatgaacagtctgcaaggtgatgacactgccatatacttctgtgccaaaagtatgattacgacgggctatactatggactactggggtcaaggaacctcagtcaccgtctcctca

SEQ ID NO: 46 - protein, 107 aa, Mus musculus
AIQMTQSSSSFSVSLGDRVTITCKASEDIYNRLAWYQQKSGNAPRLLISGSTSLETGVPSRFSGSGSGKDYTLSIISLQTEDVATYYCQQFWSTPWTFGGGTKLEIK

SEQ ID NO: 47 - protein, 11 aa, Mus musculus
KASEDIYNRLA

SEQ ID NO: 48 - protein, 7 aa, Mus musculus
GSTSLET

SEQ ID NO: 49 - protein, 9 aa, Mus musculus
QQFWSTPWT

SEQ ID NO: 50 - DNA, 321 bp, Mus musculus
gccatccagatgacacaatcttcatcctccttttctgtatctctaggagacagagtcaccattacttgcaaggcaagtgaggacatatataatcggttagcttggtatcagcagaaatcaggaaatgctccaaggctcttaatatctggttcaaccagtttggaaactggggttccttcaagattcagtggcagtggatctggaaaggattacactctcagcattatcagtcttcagactgaagatgttgctacttattactgtcaacagttttggagtactccgtggacgttcggtggaggcaccaagctggaaataaaa

SEQ ID NO: 51 - protein, 119 aa, Mus musculus
QVQLKQSGPGLVQPSQSLSITCTVSGFSLTSYGVHWVRQSPGKGLEWLGVMWRGGSTDYNAPFMSRLSITKDNSKSQVFFKMNSLQADDTAIYYCAKSLITTGYAMDYWGQGTSVTVSS

SEQ ID NO: 52 - protein, 6 aa, Mus musculus
TSYGVH

SEQ ID NO: 53 - protein, 16 aa, Mus musculus
VMWRGGSTDYNAPFMS

SEQ ID NO: 54 - protein, 11 aa, Mus musculus
SLITTGYAMDY

SEQ ID NO: 55 - DNA, 357 bp, Mus musculus
caggtccagctgaagcagtcaggacctggcctagtgcagccctcacagagcctgtccataacctgcacagtctctggtttctcattaactagctatggtgtacactgggttcgccagtctccaggaaagggtctggagtggctgggagtgatgtggagaggtggaagcacagactacaatgcacctttcatgtccagactgagcatcaccaaggacaactccaagagccaagttttctttaaaatgaacagtctgcaagctgatgacactgccatatactactgtgccaaaagtctgattacgactgggtatgctatggactactggggtcaaggaacctcagtcaccgtctcctca

SEQ ID NO: 56 - protein, 107 aa, Mus musculus
DIQMTQSSSSFSVSLGDRVTITCKASEDIYNRLAWYQQKPGNAPRLLISGATSLETGVPSRFSGSGSGKDYTLSITSLQTEDVATYYCQQYWSNPYTFGGGTKLEIK

SEQ ID NO: 57 - protein, 11 aa, Mus musculus
KASEDIYNRLA

SEQ ID NO: 58 - protein, 7 aa, Mus musculus
GATSLET

SEQ ID NO: 59 - protein, 9 aa, Mus musculus
QQYWSNPYT

SEQ ID NO: 60 - DNA, 321 bp, Mus musculus
gacatccagatgacacaatcttcatcctccttttctgtatctctaggagacagagtcaccattacttgcaaggcaagtgaggacatatataatcggttagcctggtatcagcagaaaccaggaaatgctcctaggctcttaatatctggtgcaaccagtttggaaactggggttccttcaagattcagtggcagtggatctggaaaggattacactctcagcattaccagtcttcagactgaagatgttgctacttattactgtcaacagtattggagtaatccgtacacgttcggaggggggaccaagctggaaataaaa

SEQ ID NO: 61 - protein, 120 aa, Mus musculus
QAYLQQSGAELVRSGASVKMSCKASGYTFSSYNMHWVKQTPGQGLEWIGYVYPGNGGTNYNQKFKGKATLTADTSSSTAYMQISSLTSEESAVYFCARPESSFHPWFAYWGQGTLVTVAP

SEQ ID NO: 62 - protein, 6 aa, Mus musculus
SSYNMH

SEQ ID NO: 63 - protein, 17 aa, Mus musculus
YVYPGNGGTNYNQKFKG

SEQ ID NO: 64 - protein, 11 aa, Mus musculus
PESSFHPWFAY

SEQ ID NO: 65 - DNA, 360 bp, Mus musculus
caggcttatctacagcagtctggggctgagctggtgaggtctggggcctcagtgaagatgtcctgcaaggcttctggctacacattttccagttacaatatgcactgggtaaagcagacacctggacagggcctggaatggattggatatgtttatcctggaaatggtggtacgaactacaatcagaagttcaagggcaaggccacattgactgcagacacatcctccagcacagcctacatgcagatcagcagcctgacatctgaagagtctgcggtctatttctgtgcaagaccagagagtagtttccacccctggtttgcttactggggccaagggactctggtcactgtcgcgcca

SEQ ID NO: 66 - protein, 111 aa, Mus musculus
NIVLTQSPASLAVSLGQRATISCRASESVNIYGNIFMYWYQQKPGQPPKLLIYLASNLASGVPARFSGSGSRTGFTLTIDPVEADDAATYYCQQNAKDPWTFGGGTKLEIK

SEQ ID NO: 67 - protein, 15 aa, Mus musculus
RASESVNIYGNIFMY

SEQ ID NO: 68 - protein, 7 aa, Mus musculus
LASNLAS

SEQ ID NO: 69 - protein, 9 aa, Mus musculus
QQNAKDPWT

SEQ ID NO: 70 - DNA, 333 bp, Mus musculus
aacattgtgctgacccaatctccagcttctttggctgtgtctctagggcagagggccaccatatcctgcagagccagtgaaagtgttaatatttatggcaatatttttatgtactggtaccagcagaaaccaggacagccacccaaactcctcatctatcttgcatccaacctagcatctggggtccctgccaggttcagtggcagtgggtctaggacaggcttcaccctcaccattgatcctgtggaggctgatgatgctgcaacctattactgtcagcaaaatgctaaggatccgtggacgttcggtggaggcaccaagctggaaatcaaa

SEQ ID NO: 71 - protein, 119 aa, Mus musculus
QVQLKQSGPGLVQPSQSLSITCTVSGFSLTSYGIHWVRQSPGKGLEWLGVMWRGGSTDYNAPFMSRLSITKDNSKSQVFFKMNSLQADDTAIYYCAKSLITTGYAMDFWGQGTSVTVSS

SEQ ID NO: 72 - protein, 6 aa, Mus musculus
TSYGIH

SEQ ID NO: 73 - protein, 16 aa, Mus musculus
VMWRGGSTDYNAPFMS

SEQ ID NO: 74 - protein, 11 aa, Mus musculus
SLITTGYAMDF

SEQ ID NO: 75 - DNA, 357 bp, Mus musculus
caggtccagctgaagcagtcaggacctggcctagtgcagccctcacagagcctgtccataacctgcacagtctctggtttctcattaactagctatggtatacactgggttcgccagtctccaggaaagggtctggagtggctgggagtgatgtggagaggtggaagcacagactacaatgcaccttttatgtccagactgagcatcaccaaggacaactccaagagccaagttttctttaaaatgaacagtctgcaagctgatgacactgccatatattactgtgccaaaagtctgattacgactgggtatgctatggacttctggggtcaaggaacctcagtcaccgtctcctca

SEQ ID NO: 76 - protein, 107 aa, Mus musculus; identical to SEQ ID NO: 56

SEQ ID NO: 77 - protein, 11 aa, Mus musculus
KASEDIYNRLA

SEQ ID NO: 78 - protein, 7 aa, Mus musculus
GATSLET

SEQ ID NO: 79 - protein, 9 aa, Mus musculus
QQYWSNPYT

SEQ ID NO: 80 - DNA, 321 bp, Mus musculus; identical to SEQ ID NO: 60

SEQ ID NO: 81 - protein, 119 aa, Mus musculus
QVQLKQSGPSPVQPSQSLSITCTVSGFSLTSYGVHWVRQSPGKGLEWLGVIWRGGSTDYNAVFMSRLSITKDNSKSQVFFKMNSLQTDDTAIYYCAKSLVTTGYTMDYWGQGTSVTVSS

SEQ ID NO: 82 - protein, 6 aa, Mus musculus
TSYGVH

SEQ ID NO: 83 - protein, 16 aa, Mus musculus
VIWRGGSTDYNAVFMS

SEQ ID NO: 84 - protein, 11 aa, Mus musculus
SLVTTGYTMDY

SEQ ID NO: 85 - DNA, 357 bp, Mus musculus
caggtccagctgaagcagtcaggacctagcccagtgcagccctcacagagcctgtccataacctgcacagtctctggtttctcattaacgagctatggggtacactgggttcgccagtctccaggaaagggtctggagtggctgggagtgatatggagaggtggaagtacagactacaatgcagttttcatgtccagactgagcatcaccaaggacaactccaagagccaagttttctttaaaatgaacagtctgcaaactgatgacactgccatatactactgtgccaaaagtcttgttactacgggctatactatggactactggggtcaaggaacctcagtcaccgtctcctca

SEQ ID NO: 86 - protein, 107 aa, Mus musculus
DIQMTQSSSSFSVSLGDRVTITCKASEDIYNRLAWYQQRPGNAPRLLISGTTSLETGVPSRFSGSGSGKDYTLSITSLQTEDVATYYCQQYWSIPWTFGGGTKLEIK

SEQ ID NO: 87 - protein, 11 aa, Mus musculus
KASEDIYNRLA

SEQ ID NO: 88 - protein, 7 aa, Mus musculus
GTTSLET

SEQ ID NO: 89 - protein, 9 aa, Mus musculus
QQYWSIPWT

SEQ ID NO: 90 - DNA, 321 bp, Mus musculus
gacatccagatgacacagtcttcatcctccttttctgtatctctaggagacagagtcaccattacttgcaaggcaagtgaggacatatataatcggttagcctggtatcagcagagaccaggaaatgctcctaggctcttaatatctggtacaaccagtttggaaactggggttccttcaagattcagtggcagtggatctggaaaggattacactctcagcattaccagtcttcagactgaagatgttgctacttattactgtcaacagtattggagtattccgtggacgttcggtggaggcaccaagctggaaataaaa

SEQ ID NO: 91 - protein, 119 aa, Mus musculus
QVQLKQSGPGLVQPSQSLSIICTVSGFSLSSYGIHWVRQSPGKGLEWLGVIWRGGNTDYSVTFMSRLSITKDNSKSQVFFKMNSLQADDTAIYYCAKSLITTGYAMDYWGQGTSVTVSS

SEQ ID NO: 92 - protein, 6 aa, Mus musculus
SSYGIH

SEQ ID NO: 93 - protein, 16 aa, Mus musculus
VIWRGGNTDYSVTFMS

SEQ ID NO: 94 - protein, 11 aa, Mus musculus
SLITTGYAMDY

SEQ ID NO: 95 - DNA, 357 bp, Mus musculus
caggtccagctgaagcagtcaggacctggcctagtgcagccctcacagagcctgtccataatctgcacagtctctggtttctccttaagtagttatggtattcactgggttcgccagtctccaggaaagggtctggagtggctgggagtgatatggagaggtggaaacacagactacagtgtaactttcatgtccagactgagcatcaccaaggacaactccaagagccaagttttctttaaaatgaacagtctgcaagctgatgacactgccatatattattgtgccaaaagtttgattacgacagggtatgctatggactactggggtcaaggaacctcagtcaccgtctcctca

SEQ ID NO: 96 - protein, 107 aa, Mus musculus
DIQMTQSSSSFSVSLGDRVTITCKASEDIYNRLAWYQQKPGTTPRLLISGATSLETGVPSRFSGSGSGKDYTLSITSLQTEDVGTYYCQQYWSNPRTFGGGTKLEIK

SEQ ID NO: 97 - protein, 11 aa, Mus musculus
KASEDIYNRLA

SEQ ID NO: 98 - protein, 7 aa, Mus musculus
GATSLET

SEQ ID NO: 99 - protein, 9 aa, Mus musculus
QQYWSNPRT

SEQ ID NO: 100 - DNA, 321 bp, Mus musculus
gacatccagatgacacaatcttcatcctccttttctgtttctctaggagacagagtcaccattacttgcaaggcaagtgaagacatatataatcggttagcctggtatcagcagaaaccaggaactactcctcggctcttaatatctggtgcaaccagtttggaaactggggttccttcaagattcagtggcagtggatctggaaaagattacactctcagcattaccagtcttcagactgaagatgttggtacttattactgtcaacagtattggagtaatccgcggacgttcggtggaggcaccaagctggaaataaaa

SEQ ID NO: 101 - protein, 119 aa, Mus musculus
QVQLKQSGPGLVQPSQTLSITCTVSGFSLTSYGVHWVRQSPGKGLEWLGVIWRGGGTEYNAAFMSRLSITKDNSKSQVFLKMNSLQADDTAIYYCAKSMITTGYAMDFWGQGTSVTVSS

SEQ ID NO: 102 - protein, 6 aa, Mus musculus
TSYGVH

SEQ ID NO: 103 - protein, 16 aa, Mus musculus
VIWRGGGTEYNAAFMS

SEQ ID NO: 104 - protein, 11 aa, Mus musculus
SMITTGYAMDF

SEQ ID NO: 105 - DNA, 357 bp, Mus musculus
caggtccagctgaagcagtcaggacctggcctagtgcagccctcacagaccctgtccataacctgcacagtctctggtttctcattaactagctatggtgtacactgggttcgccagtctccaggaaagggtctggagtggctgggagtgatatggagaggtggaggcacagagtacaatgcagctttcatgtccagactgagcatcaccaaggacaactccaagagccaagttttcttaaagatgaacagtctgcaagctgatgacactgccatatactactgtgccaagtcgatgattacgacaggttatgctatggacttctggggtcaaggaacctcagtcaccgtctcctca

SEQ ID NO: 106 - protein, 107 aa, Mus musculus
DIQMTQSSSSFSVSLGDRVTITCKASEDIYKRLAWYQQKPGNAPRLLISGATSLETGIPSRFSGSGSGKDYTLSLTSLQTEDVATYYCQQYWSSPRTFGGGAKLEIK

SEQ ID NO: 107 - protein, 11 aa, Mus musculus
KASEDIYKRLA

SEQ ID NO: 108 - protein, 7 aa, Mus musculus
GATSLET

SEQ ID NO: 109 - protein, 9 aa, Mus musculus
QQYWSSPRT

SEQ ID NO: 110 - DNA, 321 bp, Mus musculus
gacatccagatgacacaatcttcatcctccttttctgtatctctaggagacagagtcaccatcacttgcaaggcaagtgaggacatatataagcggttagcctggtatcagcagaaaccaggaaatgctcctaggctcttaatatctggtgcaaccagtttggaaactgggattccttcaagattcagtggcagtggatctggaaaggattacactctcagccttaccagtcttcagactgaagatgttgctacttattactgtcaacagtattggagttctcctcggacgttcggtggaggcgccaagctggaaataaaa

SEQ ID NO: 111 - protein, 119 aa, Mus musculus
QVQLKQSGPGLVQPSQSLSITCTVSGFSLTTYGIHWIRQSPGRGLEWLGVIWRGGSTDYNAAFMSRLSFTKDNSKSQVFFKMNSLQADDTAIYYCAKSMITTGFTMDYWGQGTSVTVSS

SEQ ID NO: 112 - protein, 6 aa, Mus musculus
TTYGIH

SEQ ID NO: 113 - protein, 16 aa, Mus musculus
VIWRGGSTDYNAAFMS

SEQ ID NO: 114 - protein, 11 aa, Mus musculus
SMITTGFTMDY

SEQ ID NO: 115 - DNA, 358 bp, Mus musculus
caggtccagctgaagcagtcaggacctggcctagtgcagccctcacagagcctgtccataacctgcacagtctctggtttctcattgactacctatggtatacactggattcgccagtctcctggaaggggtctggagtggctgggagtgatttggagaggtggaagcacagactacaatgcagctttcatgtccagactgagcttcaccaaggacaactccaagagccaagttttctttaaaatgaacagtctgcaagctgatgacactgccatatactactgtgccaaaagtatgattacgactggatttactatggactactggggtcaaggaacctcagtcaccgtctcctcag

SEQ ID NO: 116 - protein, 107 aa, Mus musculus
DIQMTQSSSSFSVSLGDRVTITCRASEDIYNRLAWYQQKPGNAPRLLISGATSLETGVPSRFSGSGSGKDYTLSITSLQTEDVATYYCQQFWSTPYTFGGGTKLEIK

SEQ ID NO: 117 - protein, 11 aa, Mus musculus
RASEDIYNRLA

SEQ ID NO: 118 - protein, 7 aa, Mus musculus
GATSLET

SEQ ID NO: 119 - protein, 9 aa, Mus musculus
QQFWSTPYT

SEQ ID NO: 120 - DNA, 321 bp, Mus musculus
gacatccagatgacacaatcttcatcctccttttctgtatctctaggagacagagtcaccattacttgcagggcaagtgaggacatatataaccggttagcctggtatcagcagaaaccaggaaatgctcctaggctcttaatatctggtgcaaccagtttggaaactggggttccttcaagattcagtggcagtggatctggaaaggattacactctcagcattaccagtcttcagactgaagatgttgctacttattattgtcaacagttttggagtactccgtacacgttcggaggggggaccaagctggaaataaaa

SEQ ID NO: 121 - protein, 119 aa, Mus musculus
QVQLKQSGPGLVQPSQSLSISCSVSGFSLTSYGVHWVRQSPGKGLEWLGVIWRGGSTDYNAAFSSRLSITKDNSKSQVFFKMNSLQADDTAIYYCAKSMITTGRYFDVWGAGTTVTVSS

SEQ ID NO: 122 - protein, 6 aa, Mus musculus
TSYGVH

SEQ ID NO: 123 - protein, 16 aa, Mus musculus
VIWRGGSTDYNAAFSS

SEQ ID NO: 124 - protein, 11 aa, Mus musculus
SMITTGRYFDV

SEQ ID NO: 125 - DNA, 357 bp, Mus musculus
caggtccagctgaagcagtcaggacctggcctagtgcagccctcacagagcctgtccatatcctgctcagtctctggtttctcattaactagttatggtgtacactgggttcgccagtctccaggaaagggtctggagtggctgggagtgatatggagaggaggaagcacagactacaatgcagctttctcgtccagactgagcatcaccaaggacaactccaagagccaagttttctttaaaatgaacagtctgcaagctgatgacactgccatatattactgtgccaaatctatgattacgacggggaggtacttcgatgtctggggcgcagggaccacggtcaccgtctcctca

SEQ ID NO: 126 - protein, 107 aa, Mus musculus
DIQMTQSSSSFSVSLGDRVTITCKASEDIYNRLAWYQQKPGNAPRLLISGAASLETGIPSRFSGSGSGKNYTLSITSLQTEDVATYYCQQYWSTPWTFGGGTKLEIK

SEQ ID NO: 127 - protein, 11 aa, Mus musculus
KASEDIYNRLA

SEQ ID NO: 128 - protein, 7 aa, Mus musculus
GAASLET

SEQ ID NO: 129 - protein, 9 aa, Mus musculus
QQYWSTPWT

SEQ ID NO: 130 - DNA, 321 bp, Mus musculus
gacatccagatgacacaatcttcatcctccttttctgtatctctcggagacagagtcaccattacttgcaaggcaagtgaggacatatataatcggttagcctggtatcagcagaaaccaggaaatgctcctaggctcttaatatctggtgcagccagtttggaaactgggattccttcaagattcagtggcagtggatctggaaagaattacactctcagcattaccagtcttcagactgaagatgttgctacttattactgtcaacagtattggagtactccgtggacgttcggtggaggcaccaagctggaaataaaa

SEQ ID NO: 131 - protein, 119 aa, Mus musculus
QVQLKQSGPGLVQPSQSLSITCSVSGFSLTSYGVHWVRQSPGKGLEWLGVIWRGGSTDYNAAFMSRLSITKDNSKSQVFFKMNSLQADDTAIYYCAKSMITTGRYFDVWGAGTTVTVSS

SEQ ID NO: 132 - protein, 6 aa, Mus musculus
TSYGVH

SEQ ID NO: 133 - protein, 16 aa, Mus musculus
VIWRGGSTDYNAAFMS

SEQ ID NO: 134 - protein, 11 aa, Mus musculus
SMITTGRYFDV

SEQ ID NO: 135 - DNA, 357 bp, Mus musculus
caggtccagctgaagcagtcaggacctggcctagtgcagccctcacagagcctgtccataacctgctcagtctctggtttctcattaactagctatggtgtacactgggttcgccagtctccaggaaagggtctggagtggctgggagtgatatggagaggaggaagcacagactacaatgcagctttcatgtccagactgagcatcaccaaggacaactccaagagccaagttttctttaaaatgaacagtctgcaagctgatgacactgccatatactactgtgccaaatctatgattacgacggggaggtacttcgatgtctggggcgcagggaccacggtcaccgtctcctca

SEQ ID NO: 136 - protein, 107 aa, Mus musculus
DIQMTQSSSSFSVSLGDRVTITCKASEDIYNRLAWYQQRPGNAPRLLISGATSLETGVPSRFSGSGSGKDYTLSITSLQTEDVATYYCQQYWSAPWTFGGGTKLEIK

SEQ ID NO: 137 - protein, 11 aa, Mus musculus
KASEDIYNRLA

SEQ ID NO: 138 - protein, 7 aa, Mus musculus
GATSLET

SEQ ID NO: 139 - protein, 9 aa, Mus musculus
QQYWSAPWT

SEQ ID NO: 140 - DNA, 321 bp, Mus musculus
gacatccagatgacacaatcttcatcctccttttctgtatctctcggagacagagtcaccattacttgcaaggcaagtgaggacatatataatcggttagcctggtatcagcagagaccaggaaatgctcctaggctcttaatatctggtgcaaccagtttggaaactggggttccttcaagattcagtggcagtggctctggaaaggattacactctcagcattaccagtcttcagactgaagatgttgctacttattactgtcaacagtattggagtgctccgtggacgttcggtggaggcaccaagctggaaataaaa

SEQ ID NO: 141 - protein, 117 aa, Mus musculus
DVHLVESGGGLVQPGGSRKLSCAASGFTFSSFGMHWVRQAPERGLEWVAYISGDSNTIYYTDTVKGRFTISRDNPKNTLFLQMTSLRSEDTAMYYCAKSFDYAMDYWGQGTSVTVSS

SEQ ID NO: 142 - protein, 6 aa, Mus musculus
SSFGMH

SEQ ID NO: 143 - protein, 16 aa, Mus musculus
YISGDSNTIYYTDTVK

SEQ ID NO: 144 - protein, 8 aa, Mus musculus
SFDYAMDY

SEQ ID NO: 145 - DNA, 351 bp, Mus musculus
gatgtgcatctggtggagtctgggggaggcttagtgcagcctggagggtcccggaaactctcctgtgcagcctctggattcactttcagtagctttggaatgcactgggttcgtcaggctccagagagggggctggagtgggtcgcatatatcagtggtgacagtaataccatctactatacagacacagtgaagggccggttcaccatctccagagacaatcccaagaacaccctgttcctgcaaatgaccagtctaaggtctgaggacacggccatgtattactgtgcaaaatcgttcgactatgctatggactactggggtcagggaacctcagtcaccgtctcctca

SEQ ID NO: 146 - protein, 113 aa, Mus musculus
DIVMSQSPSSLAVSVGEKVTLSCKSSQSLLYSTNQKNYLAWYLQKPGQSPKLLIYWASTRESGVPDRFSGSGSGTDFALTINTVKAEDLAVYYCQQYYTYPYTFGGGTKLEIK

SEQ ID NO: 147 - protein, 17 aa, Mus musculus
KSSQSLLYSTNQKNYLA

SEQ ID NO: 148 - protein, 7 aa, Mus musculus
WASTRES

SEQ ID NO: 149 - protein, 12 aa, Mus musculus
YYCQQYYTYPYT

SEQ ID NO: 150 - DNA, 339 bp, Mus musculus
gacattgtgatgtcacagtctccatcctccctagctgtgtcagttggagagaaggttactttgagctgcaagtccagtcagagccttttatatagtaccaatcaaaagaactacttggcctggtacctgcagaaaccagggcagtctcctaaactgctgatttactgggcatccactagggaatctggggtccctgatcgcttctcaggcagtggatctgggacagatttcgctctcaccatcaatactgtgaaggctgaagacctggcagtttattactgtcagcaatattatacctatccgtacacgttcggaggggggaccaagctggaaataaaa

SEQ ID NO: 151 - protein, 119 aa, Mus musculus
QVQLKQSGPGLVQPSQSLSITCTVSGFSLTSYGIHWLRQSPGKGLEWLGVIWRGGSTDYNAAFMSRLSITKDNSKSQVFFKMNSLQADDTAIYYCAKMRVTTGFTMDYWGQGTSVTVSS

SEQ ID NO: 152 - protein, 6 aa, Mus musculus
TSYGIH

SEQ ID NO: 153 - protein, 16 aa, Mus musculus
VIWRGGSTDYNAAFMS

SEQ ID NO: 154 - protein, 11 aa, Mus musculus
MRVTTGFTMDY

SEQ ID NO: 155 - DNA, 357 bp, Mus musculus
caggtccagctgaagcagtcaggacctggcctagtgcagccctcacagagcctgtccataacctgcacagtctctggtttctcattaactagctacggtatacactggcttcgccagtctccaggaaagggtctggagtggctgggagtgatatggagaggtggaagcacagactacaatgcagctttcatgtccagactgagcatcaccaaggacaactccaagagccaagttttctttaaaatgaacagtctgcaagctgatgacactgccatatactactgtgccaaaatgagagttacgacggggtttactatggactactggggtcaaggaacctcagtcaccgtctcctca

SEQ ID NO: 156 - protein, 107 aa, Mus musculus; identical to SEQ ID NO: 56

SEQ ID NO: 157 - protein, 11 aa, Mus musculus
KASEDIYNRLA

SEQ ID NO: 158 - protein, 7 aa, Mus musculus
GATSLET

SEQ ID NO: 159 - protein, 9 aa, Mus musculus
QQYWSNPYT

SEQ ID NO: 160 - DNA, 321 bp, Mus musculus; identical to SEQ ID NO: 60

SEQ ID NO: 161 - protein, 118 aa, Mus musculus
QVQLQQSGPELEKPGASVKISCKASGYSFTVYNINWVKQSNGKSLEWIGNIDPYFGGTIYNQKFKDKATLTVDKSSSTAYMELKSLTSEDSAVYYCERSRGTWRFAYWGQGTLVTVSA

SEQ ID NO: 162 - protein, 6 aa, Mus musculus
TVYNIN

SEQ ID NO: 163 - protein, 16 aa, Mus musculus
NIDPYFGGTIYNQKFK

SEQ ID NO: 164 - protein, 9 aa, Mus musculus
SRGTWRFAY

SEQ ID NO: 165 - DNA, 354 bp, Mus musculus
caggtccagctgcagcagtctggacctgagttggagaagcctggcgcttcagtgaagatatcctgcaaggcttctggttactcattcactgtctacaacataaactgggtgaaacagagcaatggaaagagccttgagtggattggaaatattgatccttactttggtggtactatttacaaccagaaattcaaggacaaggccacattgactgtcgacaaatcctccagcacagcctacatggagctcaagagcctgacatctgaggactctgcagtctattactgtgaaagatcgagggggacgtggaggtttgcttactggggccaagggactctggtcactgtctctgca

SEQ ID NO: 166 - protein, 107 aa, Mus musculus
DIVMTQSPSSMSVSLGDTVTITCHASQGISSNIGWLQQKPGKSFKGLIYHGATLEDGIPSRFSGSGSGADYSLTISSLDSEDFADYYCVQYAQFPYTFGGGTKLEIK

SEQ ID NO: 167 - protein, 11 aa, Mus musculus
HASQGISSNIG

SEQ ID NO: 168 - protein, 7 aa, Mus musculus
HGATLED

SEQ ID NO: 169 - protein, 9 aa, Mus musculus
VQYAQFPYT

SEQ ID NO: 170 - DNA, 321 bp, Mus musculus
gacattgtgatgacccagtctccatcctccatgtctgtatctctgggagacacagtcaccatcacttgccatgcaagtcagggcattagcagtaatatagggtggttgcagcagaaaccagggaaatcatttaagggcctgatctatcatggagccaccttggaagatggaattccatcaaggttcagtggcagtggatctggagcagattattctctcaccatcagcagcctggactctgaagattttgcagactattactgcgtacagtatgctcagtttccgtacacgttcggaggggggaccaagctggaaatcaaa

SEQ ID NO: 171 - protein, 119 aa, Mus musculus
EVQLKQSGPGLMQPSQSLSITCTVSGFSLTSYGIHWLRQSPGKGLEWLGVIWRGGSTDYNAAFMSRLSITKDNSKSQVFFKMNSLQGDDTAIYYCAKGKVTTGFYFDFWGQGTTLTVSS

SEQ ID NO: 172 - protein, 6 aa, Mus musculus
TSYGIH

SEQ ID NO: 173 - protein, 16 aa, Mus musculus
VIWRGGSTDYNAAFMS

SEQ ID NO: 174 - protein, 11 aa, Mus musculus
GKVTTGFYFDF

SEQ ID NO: 175 - DNA, 357 bp, Mus musculus
gaggtgcagctgaagcagtcaggacctggcctaatgcagccctcacagagcctgtccataacctgcacagtctctggtttctcattaactagctatggtatacactggcttcgtcagtctccaggaaagggtctggagtggctgggagtgatatggagaggtggaagcacagactacaatgcagctttcatgtccagactgagcatcaccaaggacaactccaagagccaagttttctttaaaatgaacagtctgcaaggtgatgacactgccatatactactgtgccaaagggaaggttacgacggggttctactttgacttctggggccaaggcaccactctcacagtctcctca

SEQ ID NO: 176 - protein, 107 aa, Mus musculus
DIQMTQSSSSFSVSLGDRVTITCKASEDIYNRLVWYQQKPGNAPGLLISGVTSLETGVPSRFSGSGSGKDYTLTITSLQTEDVATYYCQQYWSTPYTFGGGTKLEIK

SEQ ID NO: 177 - protein, 11 aa, Mus musculus
KASEDIYNRLV

SEQ ID NO: 178 - protein, 7 aa, Mus musculus
GVTSLET

SEQ ID NO: 179 - protein, 9 aa, Mus musculus
QQYWSTPYT

SEQ ID NO: 180 - DNA, 321 bp, Mus musculus
gacatccagatgacacaatcttcatcctccttttctgtatctctaggagacagagtcaccattacttgcaaggcaagtgaggacatatataatcggttagtctggtatcagcagaaaccaggaaatgctcctgggctcttaatatctggtgtaaccagtttggaaactggagttccttcaagattcagtggcagtggatctggaaaggattacactctcactattaccagtcttcagactgaagatgttgctacttattactgtcaacagtattggagtactccgtacacgttcggagggggaaccaagctggaaataaaa

SEQ ID NO: 181 - protein, 119 aa, Homo sapiens
QVQLVQSGAELKKPGSSVKVSCKVSGFSLTSYGIHWLRQAPGQGLEWMGVIWRGGSTDYNAKFQGRVTITKDNSKSTVYMELSSLRSEDTAVYYCAKGKVTTGFYFDFWGQGTLVTVSS

SEQ ID NO: 182 - protein, 10 aa, Homo sapiens
GFSLTSYGIH

SEQ ID NO: 183 - protein, 16 aa, Homo sapiens
VIWRGGSTDYNAKFQG

SEQ ID NO: 184 - protein, 11 aa, Homo sapiens
GKVTTGFYFDF

SEQ ID NO: 185 - DNA, 357 bp, Homo sapiens
caggttcagctggttcagtctggcgccgagctgaagaaacctggcagctctgtgaaggtgtcctgcaaggtgtccggcttcagcctgacaagctacggcatccactggctgagacaggcccctggacaaggcttggaatggatgggagtgatttggagaggcggcagcaccgactacaacgccaagtttcagggcagagtgaccatcaccaaggacaacagcaagagcaccgtgtacatggaactgagcagcctgagaagcgaggacaccgccgtgtactactgcgccaagggcaaagtgacaaccggcttctacttcgacttctggggccagggaaccctggtcacagtctcttct

SEQ ID NO: 186 - protein, 119 aa, Homo sapiens
QVTLKESGPVLVKPTETLTLTCTVSGFSLTSYGIHWLRQPPGKALEWLAVIWRGGSTDYNASLKSRLTISKDNSKSQVVLTMTNMDPVDTATYYCAKGKVTTGFYFDFWGQGTLVTVSS

SEQ ID NO: 187 - protein, 10 aa, Homo sapiens
GFSLTSYGIH

SEQ ID NO: 188 - protein, 16 aa, Homo sapiens
VIWRGGSTDYNASLKS

SEQ ID NO: 189 - protein, 11 aa, Homo sapiens
GKVTTGFYFDF

SEQ ID NO: 190 - DNA, 357 bp, Homo sapiens
caagtgaccctgaaagaaagcggccctgtgctggtcaagcccaccgaaacactgaccctgacctgtaccgtgtccggcttcagcctgacaagctacggaatccactggctgagacagcctccaggcaaggctctggaatggctggccgttatttggagaggcggcagcacagactacaacgccagcctgaagtccagactgaccatcagcaaggacaacagcaagagccaggtggtgctgaccatgaccaacatggaccctgtggacaccgccacctactactgcgctaagggcaaagtgaccaccggcttctacttcgacttttggggccagggcaccctggtcacagtctcttct

SEQ ID NO: 191 - protein, 119 aa, Homo sapiens
EVQLLESGGGLVQPGGSLRLSCAVSGFSLTSYGIHWLRQAPGKGLEWVSVIWRGGSTDYNASVKGRFTISKDNSKSTVYLQMNSLRAEDTAVYYCAKGKVTTGFYFDFWGQGTLVTVSS

SEQ ID NO: 192 - protein, 10 aa, Homo sapiens
GFSLTSYGIH

SEQ ID NO: 193 - protein, 16 aa, Homo sapiens
VIWRGGSTDYNASVKG

SEQ ID NO: 194 - protein, 11 aa, Homo sapiens
GKVTTGFYFDF

SEQ ID NO: 195 - DNA, 357 bp, Homo sapiens
gaagtgcagctgctggaatctggcggaggactggttcaacctggcggctctctgagactgagctgtgctgtgtctggcttcagcctgaccagctacggaatccactggctgagacaggcccctggcaaaggactggaatgggtgtccgtgatttggagaggcggcagcacagactacaacgcctctgtgaagggcagattcaccatcagcaaggacaacagcaagagcaccgtgtacctgcagatgaacagcctgagagccgaggacaccgccgtgtactactgtgccaagggcaaagtgaccaccggcttctacttcgacttttggggccagggcaccctggtcacagtctcttct

SEQ ID NO: 196 - protein, 119 aa, Homo sapiens
QVQLQESGPGLVKPSETLSLTCTVSGFSLTSYGIHWLRQPPGKGLEWIGVIWRGGSTDYNASLKSRVTISKDNSKSQVSLKLSSVTAADTAVYYCAKGKVTTGFYFDFWGQGTLVTVSS

SEQ ID NO: 197 - protein, 10 aa, Homo sapiens
GFSLTSYGIH

SEQ ID NO: 198 - protein, 16 aa, Homo sapiens
VIWRGGSTDYNASLKS

SEQ ID NO: 199 - protein, 11 aa, Homo sapiens
GKVTTGFYFDF

SEQ ID NO: 200 - DNA, 357 bp, Homo sapiens
caggttcagctgcaagagtctggacctggcctggtcaagcctagcgagacactgagcctgacctgtaccgtgtctggcttcagcctgaccagctacggaatccactggctgagacagcctccaggcaaaggcctggaatggatcggagtgatttggagaggcggcagcaccgactacaacgccagcctgaagtccagagtgaccatcagcaaggacaacagcaagagccaggtgtccctgaagctgagcagcgtgacagccgctgataccgccgtgtactactgcgccaagggcaaagtgaccaccggcttctacttcgacttttggggccagggcaccctggtcacagtctcttct

SEQ ID NO: 201 - protein, 119 aa, Homo sapiens
EVQLVQSGAELKKPGESLKISCKVSGFSLTSYGIHWLRQMPGKGLEWMGVIWRGGSTDYNASFQGQVTISKDNSKSTVYLQWSSLKASDTAMYYCAKGKVTTGFYFDFWGQGTLVTVSS

SEQ ID NO: 202 - protein, 10 aa, Homo sapiens
GFSLTSYGIH

SEQ ID NO: 203 - protein, 16 aa, Homo sapiens
VIWRGGSTDYNASFQG

SEQ ID NO: 204 - protein, 11 aa, Homo sapiens
GKVTTGFYFDF

SEQ ID NO: 205 - DNA, 357 bp, Homo sapiens
gaagtgcagctggttcagtctggcgccgagctgaagaagcctggcgagagcctgaagatcagctgcaaggtgtccggcttcagcctgacctcttacggcatccactggctgagacagatgcctggcaaaggcctggaatggatgggagtgatttggagaggcggcagcaccgactacaacgccagctttcagggccaagtgaccatcagcaaggacaacagcaagagcaccgtgtacctgcagtggtccagcctgaaggccagcgacacagccatgtactactgcgccaagggcaaagtgacaaccggcttctacttcgacttctggggccagggaaccctggtcacagtctcttct

SEQ ID NO: 206 - protein, 119 aa, Homo sapiens
QVQLVQSGSELKKPGASVKVSCKVSGFSLTSYGIHWLRQAPGQGLEWMGVIWRGGSTDYNAGFTGRFVISKDNSKSTVYLQICSLKAEDTAVYYCAKGKVTTGFYFDFWGQGTLVTVSS

SEQ ID NO: 207 - protein, 10 aa, Homo sapiens
GFSLTSYGIH

SEQ ID NO: 208 - protein, 16 aa, Homo sapiens
VIWRGGSTDYNAGFTG

SEQ ID NO: 209 - protein, 11 aa, Homo sapiens
GKVTTGFYFDF

SEQ ID NO: 210 - DNA, 357 bp, Homo sapiens
caggttcagctggtgcagtctggcagcgagctgaagaaacctggcgcctctgtgaaggtgtcctgcaaggtgtccggcttcagcctgacaagctacggcatccactggctgagacaggcccctggacaaggcttggaatggatgggagtgatttggagaggcggcagcaccgattacaacgccggcttcacaggcagattcgtgatcagcaaggacaacagcaagagcaccgtgtacctgcagatctgtagcctgaaggccgaggacaccgccgtgtactactgtgccaagggcaaagtgaccaccggcttctacttcgacttttggggccagggcaccctggtcacagtctcttct

SEQ ID NO: 211 - protein, 119 aa, Homo sapiens
QVQLQESGPGLVKPSETLSLTCTVSGFSLTSYGIHWIRQPPGKGLEWIGVIWRGGSTDYNPSLKSRVTISKDNSKSQVSLKLSSVTAADTAVYYCAKGKVTTGFYFDFWGQGTLVTVSS

SEQ ID NO: 212 - protein, 10 aa, Homo sapiens
GFSLTSYGIH

SEQ ID NO: 213 - protein, 16 aa, Homo sapiens
VIWRGGSTDYNPSLKS

SEQ ID NO: 214 - protein, 11 aa, Homo sapiens
GKVTTGFYFDF

SEQ ID NO: 215 - DNA, 357 bp, Homo sapiens
caggttcagctgcaagagtctggacctggcctggtcaagcctagcgagacactgagcctgacctgtaccgtgtctggcttcagcctgaccagctacggaatccactggatcagacagcctccaggcaaaggcctggaatggatcggagtgatttggagaggcggcagcaccgactacaaccccagcctgaagtccagagtgaccatcagcaaggacaacagcaagagccaggtgtccctgaagctgagcagcgtgacagccgctgataccgccgtgtactactgcgccaagggcaaagtgaccaccggcttctacttcgacttttggggccagggcaccctggtcacagtctcttct

SEQ ID NO: 216 - protein, 119 aa, Homo sapiens
QVQLQESGPGLVKPSETLSLTCTVSGFSLTSYGIHWLRQPPGKGLEWIGVIWRGGSTDYNPSLKSRVTISKDTSKSQVSLKLSSVTAADTAVYYCAKGKVTTGFYFDFWGQGTLVTVSS

SEQ ID NO: 217 - protein, 10 aa, Homo sapiens
GFSLTSYGIH

SEQ ID NO: 218 - protein, 16 aa, Homo sapiens
VIWRGGSTDYNPSLKS

SEQ ID NO: 219 - protein, 11 aa, Homo sapiens
GKVTTGFYFDF

SEQ ID NO: 220 - DNA, 357 bp, Homo sapiens
caggttcagctgcaagagtctggacctggcctggtcaagcctagcgagacactgagcctgacctgtaccgtgtctggcttcagcctgaccagctacggaatccactggctgagacagcctccaggcaaaggcctggaatggatcggagtgatttggagaggcggcagcaccgactacaaccccagcctgaagtccagagtgaccatcagcaaggacaccagcaagagccaggtgtccctgaagctgagcagcgtgacagccgctgataccgccgtgtactactgcgccaagggcaaagtgaccaccggcttctacttcgacttttggggccagggcaccctggtcacagtctcttct

SEQ ID NO: 221 - protein, 119 aa, Homo sapiens
QVQLQESGPGLVKPSETLSLTCTVSGFSLTSYGIHWIRQPPGKGLEWIGVIWRGGSTDYNPSLKSRVTISKDTSKSQVSLKLSSVTAADTAVYYCARGKVTTGFYFDFWGQGTLVTVSS

SEQ ID NO: 222 - protein, 10 aa, Homo sapiens
GFSLTSYGIH

SEQ ID NO: 223 - protein, 16 aa, Homo sapiens
VIWRGGSTDYNPSLKS

SEQ ID NO: 224 - protein, 11 aa, Homo sapiens
GKVTTGFYFDF

SEQ ID NO: 225 - DNA, 357 bp, Homo sapiens
caggttcagctgcaagagtctggacctggcctggtcaagcctagcgagacactgagcctgacctgtaccgtgtctggcttcagcctgaccagctacggaatccactggatcagacagcctccaggcaaaggcctggaatggatcggagtgatttggagaggcggcagcaccgactacaaccccagcctgaagtccagagtgaccatcagcaaggacaccagcaagagccaggtgtccctgaagctgagcagcgtgacagccgctgataccgccgtgtactactgcgccagaggcaaagtgaccaccggcttctacttcgacttttggggccagggcaccctggtcacagtctcttct

SEQ ID NO: 226 - protein, 119 aa, Homo sapiens
QVQLQESGPGLVKPSETLSLTCTVSGFSLTSYGIHWIRQPPGKGLEWIGVIWAGGSTDYNPSLKSRVTISKDTSKSQVSLKLSSVTAADTAVYYCARGKVTTGFYFDFWGQGTLVTVSS

SEQ ID NO: 227 - protein, 10 aa, Homo sapiens
GFSLTSYGIH

SEQ ID NO: 228 - protein, 16 aa, Homo sapiens
VIWAGGSTDYNPSLKS

SEQ ID NO: 229 - protein, 11 aa, Homo sapiens
GKVTTGFYFDF

SEQ ID NO: 230 - DNA, 357 bp, Homo sapiens
caggttcagctgcaagagtctggacctggcctggtcaagcctagcgagacactgagcctgacctgtaccgtgtctggcttcagcctgaccagctacggaatccactggatcagacagcctccaggcaaaggcctggaatggatcggagtgatttgggctggcggcagcaccgactacaaccccagcctgaagtccagagtgaccatcagcaaggacaccagcaagagccaggtgtccctgaagctgagcagcgtgacagccgctgataccgccgtgtactactgcgccagaggcaaagtgaccaccggcttctacttcgacttttggggccagggcaccctggtcacagtctcttct

SEQ ID NO: 231 - protein, 119 aa, Homo sapiens
QVQLQESGPGLVKPSETLSLTCTVSGFSLTSYGIHWIRQPPGKGLEWIGVIWRGGSTDYNPSLQSRVTISKDTSKSQVSLKLSSVTAADTAVYYCARGKVTTGFYFDFWGQGTLVTVSS

SEQ ID NO: 232 - protein, 10 aa, Homo sapiens
GFSLTSYGIH

SEQ ID NO: 233 - protein, 16 aa, Homo sapiens
VIWRGGSTDYNPSLQS

SEQ ID NO: 234 - protein, 11 aa, Homo sapiens
GKVTTGFYFDF

SEQ ID NO: 235 - DNA, 357 bp, Homo sapiens
caggttcagctgcaagagtctggacctggcctggtcaagcctagcgagacactgagcctgacctgtaccgtgtctggcttcagcctgaccagctacggaatccactggatcagacagcctccaggcaaaggcctggaatggatcggagtgatttggagaggcggcagcaccgactacaaccccagcctgcagtccagagtgaccatcagcaaggacaccagcaagagccaggtgtccctgaagctgagcagcgtgacagccgctgataccgccgtgtactactgcgccagaggcaaagtgaccaccggcttctacttcgacttttggggccagggcaccctggtcacagtctcttct

SEQ ID NO: 236 - protein, 107 aa, Homo sapiens
DIQMTQSPSSLSASVGDRVTITCRASEDIYNRLVWYQQKPGKAPKLLISGVTSLETGVPSRFSGSGSGKDYTLTISSLQPEDFATYYCQQYWSTPYTFGQGTKVEIK

SEQ ID NO: 237 - protein, 11 aa, Homo sapiens
RASEDIYNRLV

SEQ ID NO: 238 - protein, 7 aa, Homo sapiens
GVTSLET

SEQ ID NO: 239 - protein, 9 aa, Homo sapiens
QQYWSTPYT

SEQ ID NO: 240 - DNA, 321 bp, Homo sapiens
gacatccagatgacacagagccctagcagcctgtctgccagcgtgggagacagagtgaccatcacctgtagagccagcgaggacatctacaacagactcgtgtggtatcagcagaagcccggcaaggctcccaagctgctgattagcggagtgaccagcctggaaacaggcgtgccaagcagattcagcggctctggctctggcaaggactacaccctgaccatctctagcctgcagcctgaggacttcgccacctactactgccagcagtactggtctaccccttacaccttcggccagggcaccaaggtggaaatcaag

SEQ ID NO: 241 - protein, 107 aa, Homo sapiens
DIVMTQSPLSLPVTPGEPASISCRASEDIYNRLVWYLQKPGQSPQLLISGVTSLETGVPDRFSGSGSGKDYTLKISRVEAEDVGVYYCQQYWSTPYTFGQGTKVEIK

SEQ ID NO: 242 - protein, 11 aa, Homo sapiens
RASEDIYNRLV

SEQ ID NO: 243 - protein, 7 aa, Homo sapiens
GVTSLET

SEQ ID NO: 244 - protein, 9 aa, Homo sapiens
QQYWSTPYT

SEQ ID NO: 245 - DNA, 321 bp, Homo sapiens
gacatcgtgatgacacagagccctctgagcctgcctgtgacacctggcgaacctgccagcatcagctgtagagccagcgaggacatctacaacagactcgtgtggtatctgcagaagcccggccagtctcctcagctgctgattagcggagtgaccagcctggaaacaggcgtgccagacagattcagcggctctggctctggcaaggactacaccctgaagatcagcagagtggaagccgaggacgtgggcgtgtactactgccagcagtactggtctaccccttacaccttcggccagggcaccaaggtggaaatcaag

SEQ ID NO: 246 - protein, 107 aa, Homo sapiens
EIVMTQSPPTLSLSPGERVTLSCRASEDIYNRLVWYQQKPGQAPRLLISGVTSLETSIPARFSGSGSGKDYTLTISSLQPEDFAVYYCQQYWSTPYTFGQGTKVEIK

SEQ ID NO: 247 - protein, 11 aa, Homo sapiens
RASEDIYNRLV

SEQ ID NO: 248 - protein, 7 aa, Homo sapiens
GVTSLET

SEQ ID NO: 249 - protein, 9 aa, Homo sapiens
QQYWSTPYT

SEQ ID NO: 250 - DNA, 321 bp, Homo sapiens
gagatcgtgatgacacagagccctccaacactgtctctgagccctggcgagagagtgaccctgagctgtagagccagcgaggacatctacaacagactcgtgtggtatcagcagaagcccggccaggctcctagactgctgattagcggagtgaccagcctggaaactagcatccccgccagattcagcggctctggctctggcaaggactacaccctgacaatcagcagcctgcagcctgaggacttcgccgtgtactactgccagcagtactggtctaccccttacaccttcggccagggcaccaaggtggaaatcaag

SEQ ID NO: 251 - protein, 107 aa, Homo sapiens
EIVMTQSPGTLSLSPGERATLSCRASEDIYNRLVWYQQKPGQAPRLLISGVTSLETGIPDRFSGSGSGKDYTLTISRLEPEDFAVYYCQQYWSTPYTFGQGTKVEIK

SEQ ID NO: 252 - protein, 11 aa, Homo sapiens
RASEDIYNRLV

SEQ ID NO: 253 - protein, 7 aa, Homo sapiens
GVTSLET

SEQ ID NO: 254 - protein, 9 aa, Homo sapiens
QQYWSTPYT

SEQ ID NO: 255 - DNA, 321 bp, Homo sapiens
gagatcgtgatgacacagagccccggcacactgtcactgtctccaggcgaaagagccacactgagctgtagagccagcgaggacatctacaacagactcgtgtggtatcagcagaagcccggccaggctcctagactgctgattagcggagtgaccagcctggaaacaggcatccccgacagattcagcggctctggctctggcaaggactacaccctgaccatcagcagactggaacccgaggacttcgccgtgtactactgccagcagtactggtctaccccttacaccttcggccagggcaccaaggtggaaatcaag

SEQ ID NO: 256 - protein, 107 aa, Homo sapiens
DIVMTQSPDSLAVSLGERATINCKASEDIYNRLVWYQQKPGQPPKLLISGVTSLETGVPDRFSGSGSGKDYTLTISSLQAEDVAVYYCQQYWSTPYTFGQGTKVEIK

SEQ ID NO: 257 - protein, 11 aa, Homo sapiens
KASEDIYNRLV

SEQ ID NO: 258 - protein, 7 aa, Homo sapiens
GVTSLET

SEQ ID NO: 259 - protein, 9 aa, Homo sapiens
QQYWSTPYT

SEQ ID NO: 260 - DNA, 321 bp, Homo sapiens
gacatcgtgatgacacagagccctgatagcctggccgtgtctctgggagagagagccaccatcaactgcaaggccagcgaggacatctacaacagactcgtgtggtatcagcagaagcccggccagcctcctaagctgctgattagcggagtgaccagcctggaaacaggcgtgccagacagattcagcggctctggctctggcaaggactacaccctgacaatcagctccctgcaggccgaggatgtggccgtgtactactgccagcagtactggtctaccccttacaccttcggccagggcaccaaggtggaaatcaag

SEQ ID NO: 261 - protein, 107 aa, Homo sapiens
EIVMTQSPPTLSLSPGERVTLSCRASEDIYNRLVWYQQKPGQAPRLLISGVTSLETSIPARFSGSGSGTDYTLTISSLQPEDFAVYYCQQYWSTPYTFGQGTKVEIK

SEQ ID NO: 262 - protein, 11 aa, Homo sapiens
RASEDIYNRLV

SEQ ID NO: 263 - protein, 7 aa, Homo sapiens
GVTSLET

SEQ ID NO: 264 - protein, 9 aa, Homo sapiens
QQYWSTPYT

SEQ ID NO: 265 - DNA, 321 bp, Homo sapiens
gagatcgtgatgacacagagccctccaacactgtctctgagccctggcgagagagtgaccctgagctgtagagccagcgaggacatctacaacagactcgtgtggtatcagcagaagcccggccaggctcctagactgctgattagcggagtgaccagcctggaaactagcatccccgccagattcagcggctctggctctggcacagactacaccctgacaatcagcagcctgcagcctgaggacttcgccgtgtactactgccagcagtactggtctaccccttacaccttcggccagggcaccaaggtggaaatcaag

SEQ ID NO: 266 - protein, 107 aa, Homo sapiens
EIVMTQSPPTLSLSPGERVTLSCRASEDIYNYLVWYQQKPGQAPRLLISGVTSLETSIPARFSGSGSGTDYTLTISSLQPEDFAVYYCQQYWSTPYTFGQGTKVEIK

SEQ ID NO: 267 - protein, 11 aa, Homo sapiens
RASEDIYNYLV

SEQ ID NO: 268 - protein, 7 aa, Homo sapiens
GVTSLET

SEQ ID NO: 269 - protein, 9 aa, Homo sapiens
QQYWSTPYT

SEQ ID NO: 270 - DNA, 321 bp, Homo sapiens
gagatcgtgatgacacagagccctccaacactgtctctgagccctggcgagagagtgaccctgagctgtagagccagcgaggacatctacaactacctcgtgtggtatcagcagaagcccggccaggctcctagactgctgattagcggagtgaccagcctggaaactagcatccccgccagattcagcggctctggctctggcacagactacaccctgacaatcagcagcctgcagcctgaggacttcgccgtgtactactgccagcagtactggtctaccccttacaccttcggccagggcaccaaggtggaaatcaag